(19) United States
(12) Patent Application Publication
(10) Pub. No.: US 2025/0186353 A1
(43) Pub. Date: Jun. 12, 2025

(54) ENTERIC COATED PITOLISANT FORMULATIONS AND METHODS OF USE

(71) Applicant: Bioprojet Pharma, Paris (FR)

(72) Inventors: Marc CAPET, Melesse (FR); Philippe ROBERT, Grenoble (FR); Olivier FINANCE, Rennes (FR); Jeanne-Marie LECOMTE, Paris (FR); Jean-Charles SCHWARTZ, Paris (FR); Elsie MELSOPP, Wilmington, NC (US); Kumaraswamy BUDUR, Chicago, IL (US); Jean-Stéphane JULIEN, Saint-Cyr-sur-Loire (FR)

(21) Appl. No.: 19/052,933

(22) Filed: Feb. 13, 2025

Related U.S. Application Data
(63) Continuation of application No. 18/917,141, filed on Oct. 16, 2024, now abandoned.
(60) Provisional application No. 63/590,678, filed on Oct. 16, 2023.
(60) Provisional application No. 63/616,039, filed on Dec. 29, 2023.
(60) Provisional application No. 63/679,270, filed on Aug. 5, 2024.

(30) Foreign Application Priority Data
Jul. 18, 2024 (EP) 24306216.3

(51) Int. Cl. A61K 9/28; A61K 9/20; A61K 31/4453

(52) U.S. Cl. CPC A61K 9/2886; A61K 9/2009; A61K 9/2013; A61K 9/2027; A61K 9/2054; A61K 9/2095; A61K 9/2846; A61K 9/2853; A61K 9/2866; A61K 9/2893; A61K 31/4453

(57) ABSTRACT

The present disclosure relates generally to dosage forms (e.g., oral dosage forms) and pharmaceutical compositions comprising a pharmaceutically active agent comprising pitolisant or a pharmaceutically acceptable salt, solvate, or hydrate thereof, an enteric coating, and optionally one or more pharmaceutically acceptable excipients; as well as methods of using the dosage forms and pharmaceutical compositions, and methods of making the same.p

### RELATED APPLICATIONS

This application claims the benefit of U.S. Provisional Application No. 63/590,678, filed on Oct. 16, 2023, U.S. Provisional Application No. 63/616,039, filed on Dec. 29, 2023, U.S. Provisional Application No. 63/679,270, filed on Aug. 5, 2024, and European Patent Application No. 24306216.3, filed on Jul. 18, 2024, the entire contents of which are each incorporated herein by reference.

### BACKGROUND

Pitolisant formulations are useful to treat many diseases and disorders, particularly sleep disorders such as excessive daytime sleepiness (EDS) and cataplexy. For example, WAKIX® (pitolisant monohydrochloride) is a prescription medicine approved for the treatment of EDS or cataplexy in adults with narcolepsy.

Existing pitolisant formulations are usually administered with a titrated dose in the first two weeks, beginning with around 8.9 mg/day that is doubled after one week, and then doubled again after a second week to reach a dose of 35.6 mg/day, which may be lowered depending on the patient's tolerance.

### SUMMARY

The present disclosure relates generally to dosage forms (e.g., oral dosage forms) and pharmaceutical compositions comprising a pharmaceutically active agent comprising pitolisant, which is represented by the structure of Formula (I):

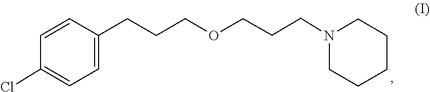 [SMILES: Clc1ccc(CCCOCCCN2CCCCC2)cc1]

or a pharmaceutically acceptable salt, solvate, or hydrate thereof, an enteric coating (also referred to as a delayed-release coating), and optionally one or more pharmaceutically acceptable excipients. Also disclosed are methods of using the dosage forms and pharmaceutical compositions (e.g., for treating a disease or disorder), as well as methods of making the same. The dosage form may further comprise an anti-moisture barrier.

An advantage of the dosage forms and pharmaceutical compositions of the present disclosure is that they can have reduced or no gastric side-effects, such as nausea, emesis, and upset stomach, when administered (e.g., orally). For example, a subject that receives by oral administration a dosage form or pharmaceutical composition disclosed herein may experience minimal or no side-effects such as nausea, upset stomach, or emesis, relative to the when the subject is orally administered an equivalent non-enterically coated dosage form or pharmaceutical composition, such as WAKIX®.

Another advantage of the dosage forms and pharmaceutical compositions of the present disclosure is they can result in improved patient compliance in the subjects receiving them, relative to patient compliance typically seen in subjects receiving a non-enterically coated dosage forms or pharmaceutical compositions, such as WAKIX®. This is due in part to the reduced side-effects experienced by subjects receiving the dosage forms or pharmaceutical compositions of the present disclosure, e.g., following oral administration. As the side-effects experienced by subjects receiving the dosage forms or pharmaceutical compositions disclosed herein may be minimal or non-existent, subjects are more likely to comply with the titration period and/or prescribed dosage regimen, and will be more likely to continue at the maximum recommended dosage, therefore receiving full clinical benefit with minimal or no side-effects.

Without wishing to be bound by theory, it is believed that the dosage forms and pharmaceutical compositions disclosed herein are bioequivalent to equivalent dosage forms comprising the same pharmaceutically active agent, without an enteric coating, such as WAKIX®. For example, orally administering a dosage form or pharmaceutical composition disclosed herein to a subject can provide a substantially similar (i.e., about the same) exposure of pitolisant compared to when orally administering an equivalent dose of WAKIX®, as determined by measuring the area under the curve (AUC) of pitolisant. Also, orally administering a dosage form or pharmaceutical composition disclosed herein can provide a substantially similar Cₘₐₓ of pitolisant, compared to when orally administering an equivalent dose of WAKIX®. Further, orally administering a dosage form or pharmaceutical composition disclosed herein can provide a substantially similar Tₘₐₓ of pitolisant, compared to when orally administering an equivalent dose of WAKIX®. This is surprising, as dosage forms comprising an enteric coating are normally expected to have a relatively delayed release of the pharmaceutically active agents, compared to their non-enterically coated counterparts, which is expected to result in non-bioequivalence e.g., due to delayed absorption of the pharmaceutically active agent and/or altered pharmacokinetics such as a blunted Cₘₐₓ, longer Tₘₐₓ, or altered AUC. Yet, dosage forms and pharmaceutical compositions of the present disclosure comprise an enteric coating and can be substantially bioequivalent to their non-enterically coated counterparts, such as WAKIX®, e.g., they do not exhibit substantially different Cₘₐₓ, Tₘₐₓ, and/or exposure as determined by AUC.

In some aspects the present disclosure relates to an oral dosage form comprising: a core; and an enteric coating that surrounds the core, wherein the core comprises pitolisant or a pharmaceutically acceptable salt, solvate, or hydrate thereof; and optionally one or more pharmaceutically acceptable excipients. The core may comprise pitolisant monohydrochloride (e.g., crystalline pitolisant monohydrochloride).

The enteric coating can comprise a polymer. The polymer may comprise an ionizable functional group, such as a carboxylic acid group. The enteric coating can comprise a polymer such as a cellulosic material (e.g., an alkylcellulose), an acrylic polymer, an acrylate polymer, a methacrylic polymer, a methacrylate polymer, or a methacrylate copolymer (e.g., an anionic methacrylate copolymer). For example, the enteric coating may comprise a EUDRAGIT® polymer, such as e.g., EUDRAGIT® L 100-55, or the enteric coating may comprise ACRYL-EZE®. It will be understood that an enteric coating may also be referred to as a delayed-release coating.

The enteric coating may further comprise a plasticizer, such as polyethylene glycol (PEG), e.g., PEG8000, or triethyl citrate.

The dosage forms of the present disclosure may comprise an anti-moisture barrier. The anti-moisture barrier can be positioned between the core and the enteric coating. For example, the dosage form may comprise a core tablet that is coated with the anti-moisture barrier (to give an anti-moisture barrier coated tablet), which itself is further coated with an enteric coating (to give an enteric-coated tablet). The anti-moisture barrier may comprise a polymer, such as polyvinyl alcohol (PVA) or a cellulose-based polymer such as HPMC. The anti-moisture barrier may comprise an OPADRY® polymer, such as OPADRY® amb II (e.g., purple OPADRY® amb II, or clear OPADRY® amb II).

The dosage form can comprise between about 1 mg and about 25 mg pitolisant or a pharmaceutically acceptable salt, solvate, or hydrate thereof (e.g., between about 3 mg and about 7 mg, or between about 18 mg and about 22 mg). For example, the dosage form can comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg of pitolisant or a pharmaceutically acceptable salt, solvate, or hydrate thereof. The pitolisant or pharmaceutically acceptable salt, solvate, or hydrate thereof, may be contained within the core of the dose form, and may not be present in the enteric coating or in the anti-moisture barrier. For example, a dosage form of the present disclosure may comprise about 5 mg of pitolisant or a pharmaceutically acceptable salt, solvate, or hydrate thereof (e.g., 5 mg of pitolisant monohydrochloride). For example, a dosage form of the present disclosure may comprise about 20 mg of pitolisant or a pharmaceutically acceptable salt, solvate, or hydrate thereof (e.g., 20 mg of pitolisant monohydrochloride).

The dosage form can comprise a pharmaceutically acceptable excipient. For example, the pharmaceutically acceptable excipient may be microcrystalline cellulose, crospovidone, talc, magnesium stearate, or colloidal silica (e.g., anhydrous colloidal silica). For example, a dosage form of the present disclosure can comprise a core, wherein the core comprises one or more of microcrystalline cellulose, crospovidone, talc, magnesium stearate, or colloidal silica (e.g., anhydrous colloidal silica).

The dosage form of the present disclosure can comprise crystalline pitolisant monohydrochloride. The dosage form may further comprise a pharmaceutically acceptable excipient. In some embodiments, the dosage form comprises a core, wherein the core comprises (e.g., consists essentially of, e.g., consists of) pitolisant monohydrochloride, microcrystalline cellulose, crospovidone, talc, magnesium stearate, and colloidal silica (e.g., anhydrous colloidal silica).

The core of the dosage form disclosed herein can be surrounded by an anti-moisture barrier that comprises (e.g., consists essentially of, e.g., consists of) a PVA-based polymer, e.g., OPADRY® amb II. The core surrounded by the anti-moisture barrier may further be surrounded by an enteric coating that comprises (e.g., consists essentially of, e.g., consists of) a copolymer of methacrylic acid and ethyl acrylate, e.g., EUDRAGIT® L 100-55 or ACRYL-EZE®.

The pitolisant or pharmaceutically acceptable salt, solvate, or hydrate thereof included in a dosage form of the present disclosure can have an X-ray diffractogram that comprises characteristic peaks (2θ) at 11.2°, 19.9°, 20.7°, and 34.1° (±0.2°). For example, the pitolisant or pharmaceutically acceptable salt, solvate, or hydrate thereof can have an X-ray diffractogram that comprises characteristic peaks (2θ) at 11.2°, 15.4°, 16.3°, 16.9°, 17.8°, 19.9°, 20.7°, 21.0°, 21.8°, 22.6°, 24.5°, 24.6°, 25.0°, 25.5°, 26.3°, 28.3°, 30.3°, 34.1°, 35.8°, 40.0°, and 46.0° (±0.2°). An exemplary X-ray diffractogram is provided in FIG. 1. In some embodiments, the pitolisant or pharmaceutically acceptable salt, solvate, or hydrate thereof present in a dosage form or pharmaceutical composition of the present disclosure has an X-ray diffractogram substantially the same as that provided in FIG. 1.

An oral dosage form of the present disclosure may be a tablet.

A dosage form of the present disclosure may be bioequivalent to a dosage form comprising pitolisant or a pharmaceutically acceptable salt, solvate, or hydrate thereof, in about the same amount, that does not comprise an enteric coating (e.g., WAKIX®).

For example, orally administering a dosage form of the present disclosure to a subject can provide a Cₘₐₓ of pitolisant in the subject that is substantially the same as the Cₘₐₓ of pitolisant obtained following orally administering an equivalent dosage form without an enteric coating (e.g., WAKIX®) to the subject. As another example, orally administering a dosage form of the present disclosure once daily for a period of about 7 days can provide a steady state Cₘₐₓ of pitolisant that is substantially the same as the steady state Cₘₐₓ of pitolisant following orally administering an equivalent dosage form without an enteric coating (e.g., WAKIX®) once daily for a period of about 7 days.

Orally administering a dosage form of the present disclosure to a subject can provide an AUC of pitolisant that is substantially the same as the AUC of pitolisant obtained following orally administering an equivalent dosage form without an enteric coating (e.g., WAKIX®) to a subject. As another example, orally administering a dosage form of the present disclosure once daily for a period of about 7 days can provide a steady state AUC of pitolisant that is substantially the same as the steady state AUC of pitolisant following orally administering an equivalent dosage form without an enteric coating (e.g., WAKIX®) once daily for a period of about 7 days.

A dosage form of the present disclosure, when orally administered to a subject, can provide a Tₘₐₓ of pitolisant that is substantially the same as the Tₘₐₓ of pitolisant obtained following orally administering an equivalent dosage form without an enteric coating (e.g., WAKIX®).

The present disclosure also relates to methods of treating a disease or disorder, comprising orally administering to a subject in need thereof an oral dosage form of the present disclosure. The disease or disorder can be a sleep disorder. For example, the disease or disorder can be excessive daytime sleepiness (EDS), cataplexy, narcolepsy, sleep apnea (e.g., obstructive sleep apnea), sleep induced apnea, or diurnal somnolence. In some embodiments, the disease or disorder is excessive daytime sleepiness (EDS). In some embodiments, the disease or disorder is cataplexy. In some embodiments, the method involves treating a disease or disorder in a subject that has narcolepsy (e.g., an adult with narcolepsy).

The present disclosure further relates to a dosage form (e.g., oral dosage form) described herein for use in the treatment of a disease or disorder, optionally, wherein the disease or disorder is excessive daytime sleepiness (EDS), cataplexy, narcolepsy, sleep apnea (e.g., obstructive sleep apnea), sleep induced apnea, or diurnal somnolence. The disease or disorder may be in a subject with narcolepsy (e.g., an adult subject with narcolepsy).

The present disclosure also relates to use of an oral dosage form described herein for the manufacture of a medicament for the treatment of a disease or disorder, optionally, wherein the disease or disorder is excessive daytime sleepiness (EDS), cataplexy, narcolepsy, sleep apnea (e.g., obstructive sleep apnea), sleep induced apnea, or diurnal somnolence. The disease or disorder may be in a subject with narcolepsy (e.g., an adult subject with narcolepsy).

The present disclosure also relates to a method of making an oral dosage form described herein. The method can comprise the steps of: (a) blending pitolisant or a pharmaceutically acceptable salt, solvate, or hydrate thereof with one or more pharmaceutically acceptable excipients (e.g., microcrystalline cellulose, crospovidone, talc, magnesium stearate, colloidal silica (e.g., anhydrous colloidal silica) or a combination thereof), to provide a blend; (b) tableting the blend (e.g., using a tableting press) to provide a core tablet; (c) optionally, coating the core tablet with an anti-moisture barrier, to provide an anti-moisture coated tablet; and (d) coating the core tablet of step (b) or the anti-moisture barrier coated tablet of step (c) with an enteric coating, to provide an enteric coated tablet.

The anti-moisture barrier optionally applied in step (c) may comprise a polymer, such as polyvinyl alcohol (PVA) or HPMC, e.g., an OPADRY® polymer, e.g., OPADRY® amb II. The enteric coating applied in step (d) may also comprise a polymer, such as a copolymer of methacrylic acid and ethyl acrylate, e.g., EUDRAGIT® L 100-55 or ACRYL-EZE®. The enteric coating applied in step (d) may also comprise a plasticizer, such as triethyl citrate or PEG (e.g., PEG8000).

### BRIEF DESCRIPTION OF DRAWINGS

FIG. 1 is an X-ray diffraction pattern of a polymorph of crystalline pitolisant monohydrochloride suitable for use in a dosage form of the present disclosure.

FIG. 2 is a plot depicting mass evolution during compression of Batch 1 core tablets.

FIG. 3 is a plot depicting hardness evolution during compression of Batch 1 core tablets.

FIG. 4 is an overlay of plots depicting stability dissolution profiles (means) of Batch 1-1 (10% GR), Batch 1-2 (12% GR), and Batch 1-3 (15% GR), each of which are exemplary enteric-coated dosage forms containing 20 mg pitolisant HCl, in baskets at 100 rpm.

FIG. 5 is an overlay of plots depicting stability dissolution profiles (means) of Batches 3-6, each of which are exemplary enteric-coated dosage forms containing 20 mg pitolisant HCl, in baskets at 100 rpm.

FIG. 6 is an overlay of plots depicting stability dissolution profiles (means) of Batch 7-1 (10% GR), Batch 7-2 (13% GR), Batch 7-3 (15% GR), and Batch 7-4 (17% GR), each of which are exemplary enteric-coated dosage forms containing 20 mg pitolisant HCl.

FIG. 7 is an overlay of plots depicting stability dissolution profiles (means) of Batch 8, an exemplary enteric-coated dosage form containing 20 mg pitolisant HCl, at release and after 1 month at 5° C., 25° C./60% RH, or 40° C./75% RH.

FIGS. 8A and 8B are plots showing the dissolution profile of an exemplary enteric-coated dosage form (clinical batch LC23120A) in two sub-lots: sub-lot 1 (FIG. 8A) and sub-lot 2 (FIG. 8B).

FIG. 9 is a plot showing the dissolution profile of an exemplary enteric-coated dosage form (clinical batch LC23120A) at release.

FIG. 10 is a plot showing the dissolution profile of an exemplary enteric-coated dosage form after 3 months under different stability testing conditions, compared with the same batch at release (clinical batch LC23120A).

### DETAILED DESCRIPTION

The present disclosure relates generally to dosage forms (e.g., oral dosage forms) and pharmaceutical compositions comprising a pharmaceutically active agent comprising pitolisant, which is represented by Formula (I):

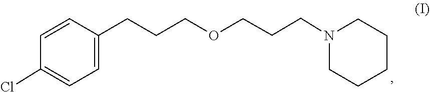 [SMILES: Clc1ccc(CCCOCCCN2CCCCC2)cc1]

or a pharmaceutically acceptable salt, solvate, or hydrate thereof, an enteric coating, and optionally one or more pharmaceutically acceptable excipients, and optionally an anti-moisture barrier coating.

The dosage forms and pharmaceutical compositions of the present disclosure, which comprise an enteric coating, have many advantages over existing pitolisant formulations such as WAKIX®. In particular, they can be tolerated more by subjects, owing to less severe or no side-effects (e.g., gastric side effects, such as stomach upset, nausea, and emesis), relative to non-enterically coated pitolisant dosage forms or pharmaceutical compositions, such as WAKIX®. Consequently, subjects receiving the dosage forms and pharmaceutical compositions of the present disclosure demonstrate better patient compliance relative to subjects receiving pitolisant formulations that do not comprise an enteric coating (e.g., WAKIX®). Another advantage of the dosage forms and pharmaceutical compositions of the present disclosure is that they are bioequivalent to WAKIX®, a pitolisant-containing dosage form approved for the treatment of EDS and cataplexy in adult subjects with narcolepsy, which is unexpected.

### Definitions

The articles “a” and “an” are used herein to refer to one or more than one (i.e., to at least one) of the grammatical object of the article. By way of example, “an element” means one element or more than one element.

The term “about” when referring to a measurable value such as an amount, a temporal duration, and the like, is meant to encompass variations of ±20% or less, or in some instances±15% or less, or in some instances±10% or less, or in some instances±5% or less, or in some instances±1% or less, or in some instances±0.1% or less, from the specified value, as such variations are appropriate.

The phrase “and/or” as used herein should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “administer,” “administering,” or “administration,” as used herein refer to implanting, absorbing, ingesting, injecting, inhaling, or otherwise introducing a compound, dosage form, or pharmaceutical composition.

The terms “comprise,” “comprises,” and “comprising” are used herein in a non-exclusive sense, except where the context requires otherwise. Likewise, the term “include” and its grammatical variants are intended to be non-limiting, such that recitation of items in a list is not to the exclusion of other like items that can be substituted or added to the listed items.

The term “effective amount” or a “therapeutically effective amount” as used herein refers to an amount of a compound, a dosage form, or a pharmaceutical composition, described herein which is sufficient to achieve a desired result under the conditions of administration. For example, an effective amount of a compound, dosage form, or pharmaceutical composition disclosed herein for treating excessive sleep disorder (EDS), e.g., in a subject with narcolepsy, is an amount that can reduce the effects of the EDS, and/or reduce or eliminate the severity of a symptom associated with the EDS. A skilled clinician can determine appropriate dosing based on a variety of considerations including the severity of the disease, the subject's age, weight, general health and other considerations. A dosage form or pharmaceutical composition disclosed herein may be administered to provide an amount of about 0.01 mg to about 250 mg (e.g., about 0.1 mg to about 100 mg) of a pharmaceutically active agent (e.g., pitolisant or a pharmaceutically acceptable salt, solvate, or hydrate thereof), e.g., about 1 mg, about 2 mg, about 3 mg, about 4 mg, about 5 mg, about 10 mg, about 15 mg, about 20 mg, about 30 mg, or about 40 mg. In some aspects, a dosage form or pharmaceutical composition of the present disclosure is administered to provide about 5 mg pitolisant hydrochloride. In some aspects, a dosage form or pharmaceutical composition of the present disclosure is administered to provide about 20 mg pitolisant hydrochloride.

The term “pharmaceutically acceptable excipient” as used herein refers to a non-toxic material that may be formulated with a compound disclosed herein to provide a pharmaceutical composition. Preferably, the pharmaceutically acceptable excipient is inert and does not interfere with the pharmacological activity of a compound which it is formulated with. Pharmaceutically acceptable excipients useful in the manufacture of the pharmaceutical compositions disclosed herein are any of those well known in the art, and include without limitation, diluents, dispersing agents, granulating agents, surface active agents, emulsifiers, disintegrating agents (sometimes referred to herein as disintegrants), binding agents (sometimes referred to herein as binders), flowing agents, preservatives, buffering agents (sometimes referred to herein as buffers), lubricating agents (sometimes referred to herein as lubricants), glidants, adjuvants, fillers, wetting agents, suspending agents, solvents, dispersion media, ion exchangers, salts, electrolytes, waxes, coloring agents, and/or oils, and the like.

For example, a pharmaceutically acceptable excipient may be alumina, a phosphate (e.g., calcium phosphate, dicalcium phosphate, tricalcium phosphate, disodium hydrogen phosphate, potassium hydrogen phosphate), a sulfate (e.g., calcium sulfate), a cellulose (including, e.g., cellulose derivatives, microcrystalline cellulose (including spray-dried microcrystalline cellulose), silicified microcrystalline cellulose, hydroxypropylmethylcellulose, methylcellulose, carboxymethylcellulose or salts thereof (e.g., sodium carboxymethylcellulose, or calcium carboxymethylcellulose)), kaolin, bentonite, VEEGUM®, lactose (e.g., anhydrous lactose, spray dried lactose, hydrated lactose), mannitol, sorbitol, sucrose, inositol, compressible sugar, trehalose, xylitol, acacia, gelatin, glucose, maltodextrin, starch (e.g., corn starch, potato starch), sodium starch glycolate, starch derivatives, an amino acid (e.g., glycine or leucine), magnesium carbonate, polyvinylpyrrolidone (PVP, povidone) (e.g., crosslinked PVP, crospovidone), polyvinyl alcohol, tragacanth, polyethylene glycol, polymethacrylates, mineral clay powders, croscarmellose, poloxamer, fatty acids or salts thereof (e.g., lauric acid, sodium lauryl sulfate, stearic acid, calcium stearate, magnesium stearate, aluminum stearate, oleic acid), hydrogenated vegetable oils, talc, titanium dioxide, glyceryl behenate, silicon dioxide (e.g., colloidal silicon dioxide), a silicate salt (e.g., magnesium trisilicate), lecithin, serum protein (e.g., human serum albumin), sorbic acid, potassium sorbate, a metal cation salt (e.g., a sodium salt, such as sodium chloride, a potassium salt, such as potassium chloride, a magnesium salt, such as or magnesium chloride, a zinc salt, such as zinc chloride), water, dimethylacetamide, protamine sulfate, a polyacrylate, wool fat, ethylenediaminetetraacetic acid (EDTA), a cyclodextrin (e.g., CAPTISOL®), KOLLIDON® CL, CELLACTOSE®, LUDIPRESS®, polysorbates (e.g., TWEEN®, e.g., TWEEN® 20 or TWEEN® 80), and combinations thereof.

The term “pharmaceutically acceptable salt” as used herein refers to salts of a compound prepared with relatively nontoxic acids or bases, depending on the particular substituents found on the respective compound. When compounds relating to the present disclosure contain relatively basic functionalities (e.g., as in Formula (I)), acid addition salts can be obtained by contacting the neutral form of such compounds with a sufficient amount of the desired acid, either neat or in a suitable solvent (e.g., an inert solvent). For example, the neutral form of pitolisant may be contacted with gaseous hydrochloric acid to provide pitolisant monohydrochloride, which can be present in a dosage form disclosed herein. Examples of pharmaceutically acceptable acid addition salts include those derived from inorganic acids like hydrochloric acid, hydrobromic acid, nitric acid, carbonic acid, monohydrogencarbonic acid, phosphoric acid, monohydrogenphosphoric acid, dihydrogenphosphoric acid, sulfuric acid, monohydrogensulfuric acid, hydriodic acid, or phosphorous acids and the like, as well as the salts derived from organic acids like acetic acid, propionic acid, isobutyric acid, maleic acid, malonic acid, benzoic acid, succinic acid, suberic acid, fumaric acid, lactic acid, mandelic acid, pamoic acid, phthalic acid, benzenesulfonic acid, p-toluenesulfonic acid, citric acid, tartaric acid, methanesulfonic acid, oxalic acid, and the like. Also included are salts of amino acids such as arginate and the like, and salts of organic acids like glucuronic or galacturonic acids and the like. Other pharmaceutically acceptable salts known to those of skill in the art are suitable for pharmaceutical compositions relating to the present disclosure.

The term “solvate” as used herein refers to forms of a compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. Compounds of the present disclosure may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” as used herein refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “subject” as used herein refers to any animal, such as any mammal, including but not limited to, humans, non-human primates, rodents, dogs, and the like. Non-human primates include chimpanzees, cynomolgus monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, feline species (e.g., domestic cat), canine species (e.g., dog, fox, wolf), avian species, and fish. In some embodiments, the subject is a mammal (e.g., a human, a rat, or a mouse). The subject can be male or female. The subject may be of any age, including an elderly human subject (e.g., 65 years or older), a human subject that is not elderly (e.g., less than 65 years old), or a human pediatric subject (e.g., less than 18 years old). In preferred aspects, the subject is a human.

As used herein, the terms “treat,” “treatment,” “treating,” or grammatically related terms, refer to a method of reducing the effects of a disease or disorder. As is readily appreciated in the art, full eradication of the disease, disorder, or symptoms thereof is preferred but not a requirement for treatment. Desirable effects of treatment include, but are not limited to, preventing occurrence or recurrence of the disease or disorder, alleviation of symptoms, diminishment of any direct or indirect pathological consequences of the disease or disorder, or other improvement of any sign, symptom, or consequence of the disease or disorder, such as prolonged survival, less morbidity, and/or a lessening of side effects.

Throughout this disclosure, various embodiments can be presented in a range format (e.g., from X-Y). It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 5, from 1 to 4, from 1 to 3, from 2 to 6, from 2 to 4, from 3 to 6, etc., as well as individual numbers within that range, e.g., 1, 2, 2.8, 3, 3.6, 4, 5, 5.4, and 6. As another example, a range such as 95-99% includes 95%, 96%, 97%, 98%, or 99% and all subranges such as 96-99%, 96-98%, 96-97%, 97-99%, 97-98%, etc. This applies regardless of the breadth of the range.

All publications (e.g., scientific journal articles, patent publications, and the like) cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

Compounds (e.g., pharmaceutically active agents) disclosed herein may also comprise one or more isotopic substitutions. For example, hydrogen (H) may be in any isotopic form, including ¹H, ²H (D or deuterium), ³H (T or tritium); carbon (C) may be in any isotopic form, including ¹²C, ¹³C, and ¹⁴C; oxygen (O) may be in any isotopic form, including ¹⁶O and ¹⁸O; nitrogen (N) may be in any isotopic form, including ¹⁴N and ¹⁵N; and chlorine (Cl) may be in any isotopic form, including ³⁵Cl and ³⁷Cl.

Various embodiments of the compounds, dosage forms, pharmaceutical compositions, and methods herein are described in further detail below, and additional definitions may be provided throughout the specification.

### Dosage Forms and Pharmaceutical Compositions

Disclosed herein are dosage forms (e.g., oral dosage forms) and pharmaceutical compositions comprising a pharmaceutically active agent and an enteric coating, and optionally one or more pharmaceutically acceptable excipients. The pharmaceutically active agent is pitolisant, represented by Formula (I):

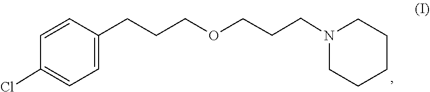 [SMILES: Clc1ccc(CCCOCCCN2CCCCC2)cc1]

or a pharmaceutically acceptable salt, solvate, or hydrate thereof.

The dosage form or pharmaceutical composition can comprise a therapeutically effective amount of the pharmaceutically active agent. For example, the dosage form or pharmaceutical composition may comprise between about 1 mg and about 200 mg of the pharmaceutically active agent, e.g., between about 1 mg and about 100 mg, between 1 mg and about 80 mg, between 1 mg and about 60 mg, between about 1 mg and about 50 mg, between about 10 mg and about 25 mg, or between about 1 mg and about 10 mg, of the pharmaceutically active agent, e.g., about 3 mg, about 4 mg, about 5 mg, about 6 mg, about 8 mg, about 10 mg, about 12 mg, about 15 mg, about 20 mg, about 25 mg, about 30 mg, about 40 mg, or about 50 mg, of the pharmaceutically active agent.

It will be understood that when the pharmaceutically active agent is a pharmaceutically acceptable salt of Formula (I), the amount of the pharmaceutically active agent in the dosage form or pharmaceutical composition will be slightly higher than the equivalent amount of free base. For example, a dosage form or pharmaceutical composition disclosed herein comprising 5 mg of pitolisant hydrochloride will comprise about 4.45 mg of pitolisant (freebase). In another example, a dosage form or pharmaceutical composition disclosed herein comprising 20 mg of pitolisant hydrochloride comprises about 17.8 mg of pitolisant (freebase). In some embodiments, a dosage form or pharmaceutical composition disclosed herein comprises about 5 mg of pitolisant monohydrochloride, or about 4.45 mg pitolisant (freebase). In some embodiments, a dosage form or pharmaceutical composition disclosed herein comprises about 20 mg of pitolisant monohydrochloride, or about 17.8 mg pitolisant (freebase).

The dosage forms of the present disclosure can be tablets, caplets, capsules, suspensions, granules, powders, or the like. Preferably, the dosage forms of the present disclosure are enteric coated tablets, enteric coated caplets, enteric coated capsules, or the like. The tablet (e.g., enteric coated tablet) may be a round, biconvex tablet. The tablet may be engraved, e.g., with a number, letter(s), or both.

The dosage forms or pharmaceutical compositions of the present disclosure can further comprise one or more pharmaceutically acceptable excipients, such as diluents, dispersing agents, granulating agents, surface active agents, emulsifiers, disintegrating agents (sometimes referred to herein as disintegrants), binding agents (sometimes referred to herein as binders), preservatives, buffering agents, lubricating agents (sometimes referred to herein as lubricants), glidants, adjuvants, fillers, wetting agents, suspending agents, solvents, dispersion media, ion exchangers, salts, electrolytes, waxes, and/or oils, and the like.

Non-limiting examples of pharmaceutically acceptable excipients suitable for use in the dosage forms or pharmaceutical compositions disclosed herein include alumina, a phosphate (e.g., calcium phosphate, dicalcium phosphate, tricalcium phosphate, disodium hydrogen phosphate, potassium hydrogen phosphate), a sulfate (e.g., calcium sulfate), cellulose, cellulose derivatives, microcrystalline cellulose (including spray-dried microcrystalline cellulose), silicified microcrystalline cellulose, hydroxypropylmethylcellulose, methylcellulose, carboxymethylcellulose or salts thereof (e.g., sodium carboxymethylcellulose, or calcium carboxymethylcellulose), kaolin, bentonite, VEEGUM®, lactose (e.g., anhydrous lactose, spray dried lactose, hydrated lactose), mannitol, sorbitol, sucrose, inositol, compressible sugar, trehalose, xylitol, acacia, gelatin, glucose, maltodextrin, starch (e.g., corn starch, potato starch), sodium starch glycolate, starch derivatives, an amino acid (e.g., glycine or leucine), magnesium carbonate, polyvinylpyrrolidone (PVP, povidone) (e.g., crosslinked PVP, crospovidone), polyvinyl alcohol, tragacanth, polyethylene glycol, polymethacrylates, mineral clay powders, croscarmellose, poloxamer, fatty acids or salts thereof (e.g., lauric acid, sodium lauryl sulfate, stearic acid, calcium stearate, magnesium stearate, aluminum stearate, oleic acid), hydrogenated vegetable oils, talc, titanium dioxide, glyceryl behenate, silicon dioxide (e.g., colloidal silicon dioxide), a silicate salt (e.g., magnesium trisilicate), lecithin, serum protein (e.g., human serum albumin), sorbic acid, potassium sorbate, a metal cation salt (e.g., a sodium salt, such as sodium chloride, a potassium salt, such as potassium chloride, a magnesium salt, such as or magnesium chloride, a zinc salt, such as zinc chloride), water, dimethylacetamide, protamine sulfate, a polyacrylate, wool fat, ethylenediaminetetraacetic acid (EDTA), a cyclodextrin (e.g., CAPTISOL®), KOLLIDON® CL, CELLACTOSE®, LUDIPRESS®, polysorbates (e.g., TWEEN®, e.g., TWEEN® 20 or TWEEN® 80), and combinations thereof.

In a preferred aspect, a dosage form (e.g., oral dosage form) or pharmaceutical composition of the present disclosure comprises a combination of pharmaceutically acceptable excipients. For example, a dosage form or pharmaceutical composition of the present disclosure may comprise one or more, or all of, the following pharmaceutically acceptable excipients: microcrystalline cellulose (e.g., Vivapur 102), crospovidone (e.g., Polyplasdone XL), talc, magnesium stearate (e.g., Kemilub EM-F-V), and colloidal silica. The pharmaceutically acceptable excipient may be present only in the core of the dosage form.

Each pharmaceutically acceptable excipient can be present in the dosage form or pharmaceutical composition in any suitable amount. For example, a pharmaceutically acceptable excipient can be present in the dosage form or pharmaceutical composition in an amount of between about 1% and about 95% by weight of the dosage form or pharmaceutical composition, e.g., between about 60% to 90%, or between about 80% to about 90%, by weight of the dosage form or pharmaceutical composition.

For example, the pitolisant may be present in the core of the dosage form in an amount of about 14 wt % to about 18 wt % (e.g., about 16 wt %), and the pharmaceutically acceptable excipients combined may account for the remainder of the weight of the core of the dosage form. One pharmaceutically acceptable excipient may be a diluent (e.g., microcrystalline cellulose) that is present in the core of the dosage form in an amount of about 60% to about 70% by weight (e.g., about 65 wt %). Another pharmaceutically acceptable excipient may be a disintegrant (e.g., crospovidone) that is present in the core of the dosage form in an amount of about 6% to about 10% by weight (e.g., about 8 wt %). Another pharmaceutically acceptable excipient may be a glidant (e.g., talc) that is present in the core of the dosage form in an amount of about 6% to about 10% by weight (e.g., about 8 wt %). Another pharmaceutically acceptable excipient may be a lubricant (e.g., magnesium stearate) that is present in the core of the dosage form in an amount of about 1% to about 4% by weight (e.g., about 2.4 wt %). Another pharmaceutically acceptable excipient may be a flowing agent (e.g., colloidal silica) that is present in the core of the dosage form in an amount of about 0.1% to about 2% by weight (e.g., about 0.8 wt %).

Preferably, the dosage forms or pharmaceutical compositions of the present disclosure are administered orally.

Preparation of the dosage forms (e.g., oral dosage forms) or pharmaceutical composition of the present disclosure can include conventional methods, such as blending, filling, compressing (e.g., direct compression, compression of dry, wet or sintered granules), coating (e.g., coating in a spray process), extrusion, granulation (e.g., wet or dry granulation), pelleting (e.g., direct pelleting), binding, powder layering (e.g., onto active ingredient-free beads, or neutral cores or particles of pharmaceutically active agent), and rounding off.

For example, a dosage form of the present disclosure may be prepared with following steps: (a) preparing a core blend comprising pitolisant, and optionally one or more pharmaceutically acceptable excipients, e.g., by blending the pharmaceutically active agent (e.g., crystalline powder) with one or more pharmaceutically acceptable excipients (e.g., binder, disintegrant, glidant, etc.). Additional sieving steps may be employed. The next step may comprise (b) forming a core tablet with the blend, e.g., by tableting using a press. Optionally, the core tablet may be coated with an anti-moisture barrier coating disclosed herein, e.g., using a spray process. Finally, the core tablet, which may be coated with an anti-moisture barrier, can be further coated with an enteric coating material disclosed herein, e.g., using a spray process. The coating steps may comprise spraying the anti-moisture barrier material or the enteric coated material as a solution, suspension, or dispersion (e.g., an organic solution, or aqueous dispersion).

### Enteric Coating

The dosage forms (e.g., oral dosage forms) and pharmaceutical compositions disclosed herein comprise an enteric coating. The enteric coating may be a material that is pH-dependent. For example, a dosage form or pharmaceutical composition disclosed herein may remain substantially intact in the stomach where the pH is low (about 1.0 to 3.5), and release the pharmaceutically active agent after entering the small intestine, due to disintegration of the enteric coating in the relatively higher pH of the small intestine (about 5.5 to 7.0). In some aspects of the present disclosure, the enteric coating may disintegrate at a higher pH, e.g., to substantially release the pharmaceutically active agent in more distal portions of the small intestine where the pH is even higher (e.g., about 6.0 to 7.5). A pharmaceutical composition or dosage form of the present disclosure may substantially release the pharmaceutically active agent in the duodenum, in the jejenum, or in both the duodenum and jejenum.

The enteric coating can be substantially resistant to disintegration in the stomach or gastric fluid, e.g., at a pH of between about 1.0 and about 3.5. The enteric coating can disintegrate in the intestine (e.g., small intestine) to expose the pharmaceutically active agent, e.g., after the dosage form or pharmaceutical composition has entered the intestine. To that end, the enteric coating may substantially disintegrate in the intestine (e.g., small intestine) or intestinal fluid, or at a pH of greater than 5 (e.g., a pH of greater than 5.5, greater than 6.0, greater than 6.5, greater than 7.0, or greater than 7.5).

An enteric coating of a dosage form or pharmaceutical composition of the present disclosure may be acid resistant (gastric resistant) and prevent more than 10 percent of the pharmaceutically active agent to be released when the dosage form is in the stomach, in a hydrochloride solution (pH 1.2), in gastric fluid, or simulated gastric fluid, for at least 1 hour (e.g., 2 hours), e.g., releasing less than 10%, less than 8%, less than 6%, less than 4% less than 3%, less than 2%, less than 1% by weight, or none of the pharmaceutically active agent. The hydrochloride solution, gastric fluid, or simulated gastric fluid may be at a temperature of about 37° C. The dosage form or pharmaceutical composition, or the enteric coating thereof, may substantially disintegrate after the dosage form or pharmaceutical composition is in the intestine (e.g., small intestine), or is exposed to intestinal fluid, simulated intestinal fluid, or a solution, e.g., buffer (e.g., phosphate buffer), with a pH of greater than 5 (e.g., a pH of greater than 5.5, 6.0, 6.5, 7.0, 7.5, or higher).

A dosage form or pharmaceutical composition according to the present disclosure may be a delayed release dosage form or pharmaceutical composition according to the United States Pharmacopeia (USP) 701 Procedure and Criteria for Delayed-Release Tablets and Capsules (tablets or capsules that are formulated with acid-resistant or enteric coatings).

An enteric coating of a dosage form or pharmaceutical composition disclosed herein may comprise a polymer. For example, the polymer of the enteric coating may be a polymer comprising an ionizable functional group (e.g., a carboxylic acid group), e.g., a functional group that is not ionized in the stomach or gastric fluid, or in a solution with a pH of between about 1.0 and about 3.5. The functional group may be a group that is ionized in the intestine (e.g., small intestine) or intestinal fluid, or in a solution with a pH of greater than 5 (e.g., a pH of greater than 5.5, greater than 6.0, greater than 6.5, greater than 7.0, or greater than 7.5).

The enteric coating may comprise an acrylic polymer, such as acrylic acid and methacrylic acid copolymers, methacrylate copolymers (e.g., copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate); a cellulosic material, such as an alkylcellulose, e.g., ethylcellulose, AQUACOAT®, SURELEASE®, hydroxypropyl methylcellulose phthalate, hydroxypropyl methylcellulose acetate succinate, cellulose acetate trimellitate, cellulose acetate phthalate, or the like; polyvinylpyrrolidones (PVP), polyvinyl alcohols, polyvinyl alcohol-polyethylene glycol graft copolymers, a KOLLICOAT® polymer, starch, starch derivatives, polyvinyl acetate (PVAc), polyvinyl acetate phthalate (PVAP, or COATERIC®), a KOLLIDON® polymer, vinyl acetate-vinylpyrrolidone copolymer (e.g., KOLLIDON® VA64), vinyl acetate: crotonic acid copolymer (VAC:CRA), polyethylene glycols (e.g., polyethylene glycols with a molecular weight above 1000 g/mol), chitosan, crosslinked and/or non-crosslinked polyacrylic acid, sodium alginate, pectin, methacrylic acid ester copolymers, ethoxyethyl methacrylates, cyanoethyl methacrylate, poly(methacrylic acid), methacrylic acid alkylamide copolymer, poly(methyl methacrylate), polymethacrylates, poly(methyl methacrylate) copolymer, polyacrylamide, aminoalkyl methacrylate copolymer, poly(methacrylic acid anhydride), glycidyl methacrylate copolymers, ammonio methacrylate copolymers, or the like; shellac, zein, or the like; a combination thereof. In some embodiments, the enteric coating comprises an anionic polymer, e.g., an anionic methacrylate copolymer. It will be understood that the polymer may not be anionic at sufficiently low pH (e.g., in gastric fluid) but becomes ionized upon increasing pH (e.g., in intestinal fluid).

The enteric coating may comprise a EUDRAGIT® polymer, such as a EUDRAGIT® L polymer, e.g., EUDRAGIT® L 30 D-55, EUDRAGIT® L 100-55, EUDRAGIT® FL 30 D-55, EUDRAGIT® L 100, EUDRAGIT® L 12,5, or a EUDRAGIT® S polymer such as EUDRAGIT® S 100, EUDRAGIT® S 12,5, EUDRAGIT® FS 30 D, EUDRAGIT® FS 100, a EUDRAGIT® RL polymer such as EUDRAGIT® RL PO, EUDRAGIT® RL 100, EUDRAGIT® RL 30D, EUDRAGIT® RL 12,5, a EUDRAGIT® RS polymer such as EUDRAGIT® RS PO, EUDRAGIT® RS 100, EUDRAGIT® RS 30 D, or EUDRAGIT® RS 12,5, a EUDRAGIT® NM polymer such as EUDRAGIT® NM 30, or a combination thereof.

The enteric coating may comprise ACRYL-EZE®, such as ACRYL-EZE® clear.

The enteric coating may also comprise a plasticizer, a stabilizer (e.g., oleic acid, or a polysorbate e.g., TWEEN®80), a lubricant, a glidant, a release agent, or a pigment (e.g., talc, titanium dioxide, or magnesium stearate). Examples of suitable plasticizers include sebacates (e.g., dibutyl sebacate), propylene glycol, polyethylene glycols (e.g., PEG8000), phthalates (e.g., diethyl phthalate or dibutyl phthalate) or phthalate esters, citric acid esters (e.g., triethyl citrate, tibutyl citrate), triacetin, acetylated monoglycerides, castor oil, propylene glycol, and polyethylene glycols. Examples of release agents include glycerol monostearate or other suitable fatty acid derivatives, silica derivatives, and talc. Preferred stabilizers are PEG8000 and triethyl citrate.

The enteric coating may comprise a material, or may be an enteric coating, disclosed in US 2004/0028737, US 2005/0271778, WO 2005/044240, WO 2007/006353, WO 2008/135090, each of which are incorporated herein by reference in their entireties.

### Anti-Moisture Barrier

The dosage forms (e.g., oral dosage forms) and pharmaceutical compositions disclosed herein can comprise an anti-moisture barrier coating. The anti-moisture barrier is distinct from the enteric coating, and the anti-moisture barrier can be present in the dosage form or pharmaceutical composition in addition to the enteric coating. For example, the anti-moisture barrier coating may be positioned around the core of a dose form and underneath the enteric coating layer (i.e., between the outer surface of the core and the inner surface of the enteric coating layer). The anti-moisture barrier may comprise any suitable material, such as material that prevents moisture from the environment readily entering the core of the dose form prior to its intended dissolution (e.g., upon administration.

The anti-moisture barrier may comprise a polymer. Suitable polymers include polyvinyl alchohols and hypromellose (hydroxypropylmethylcellulose). An exemplary anti-moisture barrier material of the present disclosure is OPADRY® amb II (e.g., purple OPADRY® amb II, or clear OPADRY® amb II).

### Pharmaceutically Active Agent

The pharmaceutically active agent of the dosage form (e.g., oral dosage form) or pharmaceutical composition disclosed herein is pitolisant (1-{3-[3-(4-chlorophenyl) propoxy]propyl}-piperidine), or a pharmaceutically acceptable salt, solvate, or hydrate thereof. Pitolisant is represented by Formula (I):

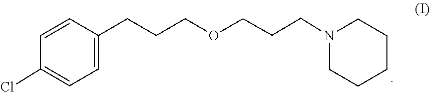 [SMILES: Clc1ccc(CCCOCCCN2CCCCC2)cc1]

The pharmaceutically active agent may be Formula (I) in freebase form, or may be any pharmaceutically acceptable salt of Formula (I), e.g., a pharmaceutically acceptable salt disclosed herein. For example, the pharmaceutically active agent can be a hydrochloride salt or an oxalate salt of Formula (I).

In preferred aspects, the pharmaceutically active agent is a hydrochloride salt of Formula (I), e.g., (1-{3-[3-(4-chlorophenyl) propoxy]propyl}-piperidine monohydrochloride; pitolisant monohydrochloride), represented by Formula (Ia):

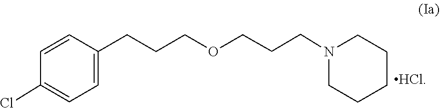 [SMILES: Clc1ccc(CCCOCCCN2CCCCC2)cc1]

Without wishing to be bound by theory, it is believed that pitolisant can activate receptors within the stomach, such as histamine H₃ receptors, which may cause gastric side effects associated with existing pitolisant formulations such as WAKIX®. The inventors discovered that by formulating pitolisant dosage forms or pharmaceutical compositions with an enteric coating, the gastric side effects can be substantially reduced or eliminated. Without wishing to be bound by theory, it is also believed that despite the enteric coating, which prevents release of pitolisant from the dosage form in the stomach or gastric fluid, there is not a blunting of Cₘₐₓ or Tₘₐₓ, relative to comparative formulations such as WAKIX®, and the overall exposure (as measured by AUC) is relatively the same between the dosage forms of the present disclosure and WAKIX®. In other words, despite their enteric coatings, the dosage forms and pharmaceutical compositions disclosed herein may be substantially bioequivalent to WAKIX®.

The dosage forms and pharmaceutical compositions disclosed herein may comprise the monohydrochloride salt of Formula (I), which has a relatively high aqueous solubility, e.g., compared to other known pharmaceutically acceptable salts of Formula (I) (e.g., pitolisant oxalate). The aqueous solubility of the pharmaceutically active agent may be about 4 g/mL at 23° C.

The pharmaceutically active agent (e.g., Formula (I), or Formula (Ia)) may be crystalline.

The pharmaceutically active agent can have a water content of 6% (±0.5%) or less, e.g., less than 5%, less than 4%, less than 3%, less than 2%, or less than 1%, by weight.

The pharmaceutically active agent can have characteristic IR peaks at 1112, 1101, 2936, 2868, 1455, 2647, 2551, 1492, and 802 cm⁻¹ (±5).

The pharmaceutically active agent can be a crystalline form of pitolisant hydrochloride.

The pharmaceutically active agent can be the crystalline form of pitolisant hydrochloride disclosed in U.S. Pat. No. 8,207,197, which is incorporated herein by reference in its entirety.

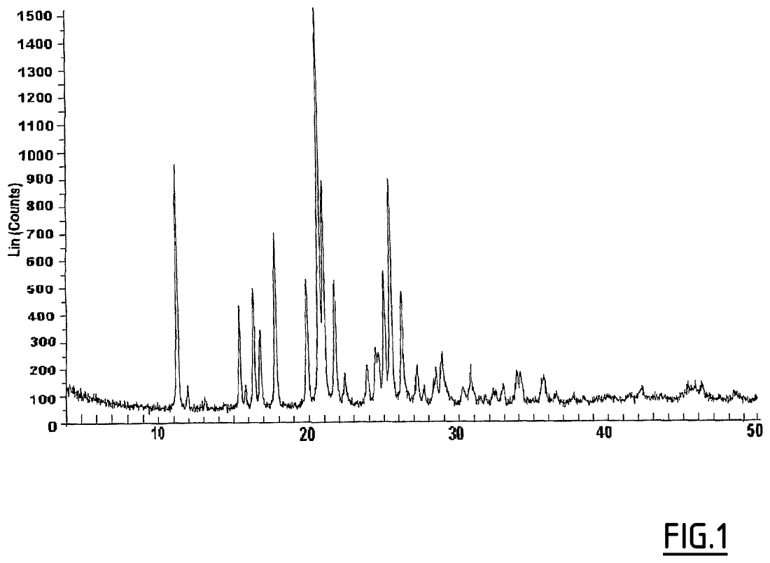

The pharmaceutically active agent can be pitolisant hydrochloride that has an X-ray powder diffraction pattern with characteristic peaks (2θ) at: 11.2°, 19.9°, 20.7° and) 34.1° (±0.2°), as obtained using X-Ray diffraction technique described in U.S. Pat. No. 8,207,197, using a Nonius Kappa Charge Coupled Device system at −158° C., operating at a wavelength of 0.71073 Å. The pharmaceutically active agent can have an X-ray powder diffraction pattern as shown in FIG. 1.

The pharmaceutically active agent may be a compound disclosed in U.S. Pat. Nos. 8,207,197, 8,354,430, 8,486,947, 7,138,413, 7,910,605, or U.S. Pat. No. 7,169,928, each of which are incorporated herein by reference in their entireties.

### Pharmacokinetics

Without wishing to be bound by theory, it is believed that the oral dosage forms and pharmaceutical compositions disclosed herein are substantially bioequivalent and/or have the substantially same bioavailability as their non-enterically coated counterparts, such as WAKIX®. In other words, and without wishing to be bound by theory, it is believed that the presence of the enteric coating of the dosage forms or pharmaceutical compositions disclosed herein does not substantially affect their pharmacokinetics and/or bioavailability, and following administration, a similar biological effect, bioavailability, and/or pharmacokinetics are achieved as would be expected by administering an equivalent dosage form or pharmaceutical composition without an enteric coating (e.g., containing the same pharmaceutically active agent and pharmaceutically acceptable excipient(s), in substantially the same amounts), such as WAKIX®.

For example, it is believed that oral administration of one or more dosage forms or pharmaceutical compositions disclosed herein to provide about 35.6 mg/day of pitolisant (freebase), can achieve a steady state Cₘₐₓ of pitolisant of between about 49.2 ng/ml and about 126 ng/ml, e.g., about 73 ng/mL. Similarly, it is believed that oral administration of one or more dosage forms or pharmaceutical compositions disclosed herein to provide about 35.6 mg/day of pitolisant (freebase), can achieve a steady state AUC of pitolisant of between about 518 ng*hr/mL and about 1468 ng*hr/mL, e.g., about 812 ng*hr/mL. It is further believed that steady state AUC and Cₘₐₓ is reached by day 7, following once daily oral administration of one or more dosage forms or pharmaceutical compositions disclosed herein.

The dosage forms (e.g., oral dosage forms) or pharmaceutical compositions disclosed herein can have substantially the same oral bioavailability as an equivalent dosage form or pharmaceutical composition without the enteric coating (e.g., WAKIX®). For example, without wishing to be bound by theory, it is believed that administering (e.g., orally administering) a dosage form or pharmaceutical composition disclosed herein can achieve an oral absorption of greater than 50%, e.g., about 75%, about 85%, about 90%, or more. It is further believed that administering (e.g., orally administering) a dosage form or pharmaceutical composition disclosed herein can achieve a Tₘₐₓ of pitolisant of between about 2 hours and about 5 hours (e.g., about 3.5 hours).

In some embodiments, the rate and/or extent of absorption of pitolisant following administration of a dosage form or pharmaceutical composition disclosed herein does not show a significant difference when compared to administering the same molar dose of the therapeutic in a dosage form that does not comprise an enteric coating, such as WAKIX®.

In some embodiments, an oral dosage form or pharmaceutical composition disclosed herein is bioequivalent to WAKIX®, as determined by the Bioequivalence Guidance 94D-0401 provided by the U.S. Food and Drug Administration (FDA).

In some embodiments, an oral dosage form or pharmaceutical composition disclosed herein has substantially the same bioavailability as WAKIX®.

### Methods of Treatment

The present disclosure further relates to a method for the treatment of a disease or disorder, comprising administering a dosage form (e.g., oral dosage form) or pharmaceutical composition disclosed herein to a subject in need thereof. The disease or disorder may be a sleep disorder (e.g., excessive daytime sleepiness (EDS), cataplexy, narcolepsy, sleep apnea (e.g., obstructive sleep apnea), sleep induced apnea, diurnal somnolence, central nervous system disorder (e.g., epilepsy, Alzheimer's disease, Parkinson's disease, dementia (e.g., dementia with Lewy bodies and/or vascular dementia), attention disorders, wakefulness disorders, memorization disorders, cognitive deficits (e.g., in aged persons), psychiatric pathologies, depressive and asthenic states, vertigo, and motion sickness, obesity, psychosomatic disorders, respiratory disorders, allergic conditions, inflammatory conditions, cardiac conditions, gastrointestinal conditions, conditions of the urogenital system, conditions of the cutaneous system, stress, migraine, headache, pain, psychotropic disorders, asthma, bronchitis, rhinitis, tracheitis, gastric ulcers, duodenal ulcers, ulcerative colitis, Crohn's disease, irritable bowel syndrome, cystitis, metritis, urinary incontinence, fecal incontinence, urticaria, itching, arthritis, conjunctivitis, premenstrual syndrome, prostatic inflammations, genital disorders, rheumatic conditions, ocular conditions, sialorrhea, convulsion, depression, disorders of the hypothalam hypophyseal system, disorders of the cerebral circulation, and disorders of the immune system.

In preferred aspects, the disease or disorder is a sleep disorder. For example, the disease or disorder can be excessive daytime sleepiness (EDS). The EDS can be in subjects (e.g., adult subjects) with narcolepsy.

The present disclosure further relates to a method for the prevention of undesirable side effects associated with using antipsychotic or antidepressant agents (e.g., aripiprazole, clozapine, olanzapine, risperidone, quetiapine, sertindole, mirtazapine, amitryptiline, and paroxetine), comprising administering a dosage form or pharmaceutical composition of the present disclosure to a subject in need thereof. Non-limiting examples of undesirable side effects associated with using antipsychotic or antidepressant agents includes weight gain, somnolence, and cognitive impairment.

The present disclosure further relates to a method for (i) inducing an extended state of wakefulness; (ii) improving cognitive processes; (iii) reducing food intake; and/or (iv) normalizing vestibular reflexes, comprising administering a dosage form or pharmaceutical composition disclosed herein to a subject in need thereof.

A dosage form (e.g., oral dosage form) or pharmaceutical composition disclosed herein may be taken once daily, twice daily, or more often. More than one dosage form can be administered at once to achieve a desired dose. Preferably, the dosage form (or dosage forms, when multiple dosage forms are used) or pharmaceutical composition is/are taken once daily. For example, one or more dosage forms disclosed herein may be administered orally once daily, e.g., in the morning upon wakening. One or more dosage forms or pharmaceutical compositions disclosed herein may be taken with a frequency (e.g., once daily) so that the total amount of pharmaceutically active agent administered is within the range of from about 10 mg to about 50 mg per day, e.g., about 15 mg to about 40 mg per day. One or more dosage forms or pharmaceutical compositions disclosed herein may be taken with a frequency (e.g., once daily) so that the total amount of pitolisant (in terms of freebase) administered is within the range of from about 17.8 mg to about 35.6 mg per day. For example, a subject may be administered orally two dosage forms once daily, where each dosage form comprises 4.45 mg pitolisant (in terms of freebase), to achieve a daily dose of 8.9 mg pitolisant (in terms of freebase). A subject may be administered orally one dosage form once daily, where the dosage form comprises 17.8 mg pitolisant (in terms of freebase), to achieve a daily dose of 17.8 mg pitolisant (in terms of freebase). A subject may be administered orally two dosage forms once daily, where each dosage form comprises 17.8 mg pitolisant (in terms of freebase), to achieve a daily dose of 35.6 mg pitolisant (free base).

### EXAMPLES

In order that the invention described herein may be more fully understood, the following examples are set forth. These examples are offered to illustrate the dose forms (e.g., enteric-coated dose forms), methods of use, and methods of making, and are not to be construed in any way as limiting their scope.

The compounds and dose forms provided herein can be prepared from readily available starting materials using modifications to the specific protocols set forth below that would be well known to those of ordinary skill in the art. It will be appreciated that where typical or preferred process conditions are given, other process conditions can also be used unless otherwise stated. Optimum reaction conditions may vary with the particular reactants or solvents used, but such conditions can be determined by those skilled in the art by routine optimization.

### Materials and Methods

Materials used in the examples set forth below were purchased from commercial suppliers. Pitolisant hydrochloride (API) was obtained from Corden Pharma Chenove (MA-22-013). Pitolisant hydrochloride may also be prepared according to the methods described in U.S. Pat. No. 8,207,197, which is incorporated herein by reference in its entirety. Microcrystalline cellulose (Vivapur 102) (diluent) was obtained from JRS Pharma (ME-19-093; ME-19-077). Crospovidone (Polyplasdone XL) (disintegrant) was obtained from BASF (0002356295). Talc (Luzenac pharma) (glidant) was obtained from IMI Fabi (TE142). Magnesium stearate (Kemilub EM-F-V) (lubricant) was obtained from Peter Greven (241231). Anhydrous colloidal silica (Aerosil 200 Pharma) (flowing agent) was obtained from Evonik (150061214). OPADRY® amb II clear (anti-moisture barrier agent) was obtained from Colorcon (TKL53663). ACRYL-EZE® Clear (gastro resistant coating agent) was also obtained from Colorcon (TKL64755). Top mill red 240.17 (coloring agent) was obtained from Biogrund (9030806-L). Water for injection (solvent) was obtained from Lavoisier (19KK18GC) (purified water can also be used). Triethyl citrate (TEC) and PEG8000 (plasticizers) were obtained from Colorcon (DTR527487 and DTR550141).

Weighing scales used included OGAL075, OGAL023, OGAL367, AND OGAL351-1. Blending used Soneco blender OGAL112, Soneco container 100 L OGAL108, and 800 μm manual sieve OGAL211-IV3. Compression used a tableting machine FETTE P1200 OGAL035-1, Turret B OGAL035-2, Punches and die D7.5R9.2, D7.5 B132 I/S/M, engraving “20.” Coating used Manesty OGAL117-1, 330 mm perforated pan OGAL117-4, nozzle 0.8 mm, pump OGAL236, agitator OAMA092, adequate beaker, tubes 4.8-2.4, and magnetic stirrer.

### Example 1. Preparation of Exemplary Pitolisant Core Tablets (Batch 1 Core)

### Manufacturing Formula

Core tablets were prepared using the manufacturing formula described in Table 1.

TABLE 1
Manufacturing formulae for core tablets for Batch 1.

| | | | | Theoretical | Actual |
|---|---|---|---|---|---|
| | | Unit formula | Formula | quantity | quantity |
| Raw material | Batch No. | (mg/unit) | (%) | (g) | (g) |
| Pitolisant HCl | MA-22-013 | 20 | 16 | 1440 | 1440.1 |
| Microcrystalline | ME-19-093 | 81 | 64.8 | 5832 | 5832 |
| cellulose | ME-19-077 | | | | |
| Crospovidone | 0002356295 | 10 | 8 | 720 | 720 |
| Talc | TE142 | 10 | 8 | 720 | 720 |
| Colloidal silica | 150061214 | 1 | 0.8 | 72 | 72 |
| Magnesium | 241231 | 3 | 2.4 | 216 | 216 |
| stearate | | | | | |
| Total | — | 125 | 100 | 9000 | 9000.1 |

### Blending Step

Prior to tableting the manufacturing formula was subjected to sieving and blending steps. Table 2. Provides the manufacturing parameters used during these steps to provide the final blend for tableting.

TABLE 2
Blending parameters

| | | Required | |
|---|---|---|---|
| Blending step | Parameters | Value | Batch 1 |
| Sieving 1 | Sieve aperture | 800 μm | 800 μm |
| ½ cellulose + | | | |
| crospovidone + | | | |
| talc + | | | |
| pitolisant HCl + | | | |
| ½ cellulose | | | |
| Blending 1 | Blending time | 3 min 38 sec | 3 min 38 sec |
| Sieved material | Blending speed | 22 rpm | 22 rpm |
| | Rotation number | 80 | 80 |
| Sieving 2 | Sieve aperture | 800 μm | 800 μm |
| Aerosil + | | | |
| magnesium | | | |
| sterate | | | |
| Lubrication | Blending time | 3 min 38 sec | 3 min 38 sec |
| Blending 1 + | Blending speed | 22 rpm | 22 rpm |
| aerosil + | Rotation number | 80 | 80 |
| magnesium | Filling level | — | 50% |
| stearate | | | |

### Manufacturing Formula

The final blend was tested for its flowability and density (Table 3). For comparison, a reference batch blend (Batch 2) was measured. Despite the appearance of poor flowability in Batch 1, there were no flowing issues during the compression step described below.

TABLE 3
IPC on final blends

| Batch Number | Batch 1 | Batch 2 |
|---|---|---|
| Sample size (g) | 50.105 | |
| Flowability | | |
| Flowing time (s) | Infinite¹ | Infinite |
| Flowing speed (g/s) | NA | 0 |
| Tapped volume (mL) | | |
| V0 | 128 | 118 |
| V10 | 118 | 114 |
| V500 | 96 | 94 |
| V1250 | 94 | 92 |
| V2500 | 92 | / |
| V10-V500 | 22 | 20 |
| Density (g/mL) | | |
| D0 | 0.39 | 0.42 |
| D10 | 0.42 | 0.44 |
| D500 | 0.52 | 0.53 |
| D1250 | 0.53 | 0.54 |
| D2500 | 0.54 | / |
| Carr index | 28 | 22 |
| Hausner ratio | 1.39 | 1.28 |
| Flow character | Poor | Passable |
| LOD (%) | 3.9 | 4.69 |

NA: not applicable;
¹Blend flows when regularly tapping on funnel

### Tableting

Batch 1 tablets were manufactured on a rotative tableting press using 5 punches D7.5R9.2 engraved with “20.” Main compression force and filling volume were adapted to reach 50N hardness tablets and 125.0 mg tablet mass. Tableting parameters are provided in Table 4.

TABLE 4
Tableting parameters and IPC results

| | | Batch 1 |
|---|---|---|
| | Specifications | D7.5R9.2 “20” |
| Tableting actual parameters | | |
| Tableting speed (tab/min) | / | 90 000 tblt/h |
| | | (=18750 |
| | | tblt/h with |
| | | 5 punches) |
| Compression force (kN) | / | 6.2 |
| Pre-compression force (kN) | / | 1.5 |
| Filling height (mm) | / | 5.98 |
| Distributor speed (%) | / | 20 |
| IPC results on n = 20 tablets | | |
| Mean tablet mass (mg) | 125.0 ± 3.0% | 125.0 |
| | (121.3-128.8) | |
| Min-Max mean tablet | T1: 7.5% | 124.1-126.0 |
| mass (mg) | (115.6-134.4) | |
| Min-Max individual | T2: 15% | 121.1-128.2 |
| tablet mass (mg) | (106.3-143.8) | |
| CV (%) | / | 0.95 |
| Mean tablet hardness (N) | / | 54 |
| Min-Max mean hardness | / | 53-55 |
| Min-Max individual hardness | / | 45-63 |
| CV (%) | / | 6.04 |
| Mean tablet thickness (mm) | / | 3.08 |
| Min-Max mean thickness | / | 3.04-3.10 |
| Min-Max individual thickness | / | 3.02-3.13 |
| CV (%) | / | 0.35 |
| Mean tablet diameter (mm) | / | 7.58 |
| Min-Max mean diameter | / | 7.57-7.59 |
| Min-Max individual diameter | / | 7.51-7.66 |
| CV (%) | / | 0.45 |
| Aspect | | Round white |
| | | tablets |
| | | with one face |
| | | marked “20” |
| Bulk density (g/mL) | / | 0.73 |

### Disintegration and Friability Tests

The Batch 1 tablets were then subjected to disintegration and friability tests, the results of which are provided in Table 5.

TABLE 5
Disintegration and Friability Test Results of Batch 1 tablets.

| | | | Representative |
|---|---|---|---|
| | | Settings | sample |
| Disintegration time [min-s] | Min | 1 min 50 sec | 3 min 13 sec |
| Specification: <15 min | Max | 3 min 33 sec | 4 min 11 sec |
| Friability [%] | | 0.1% | 0.0 (=0.05%) |
| Specification: ≤1.0% | | | |

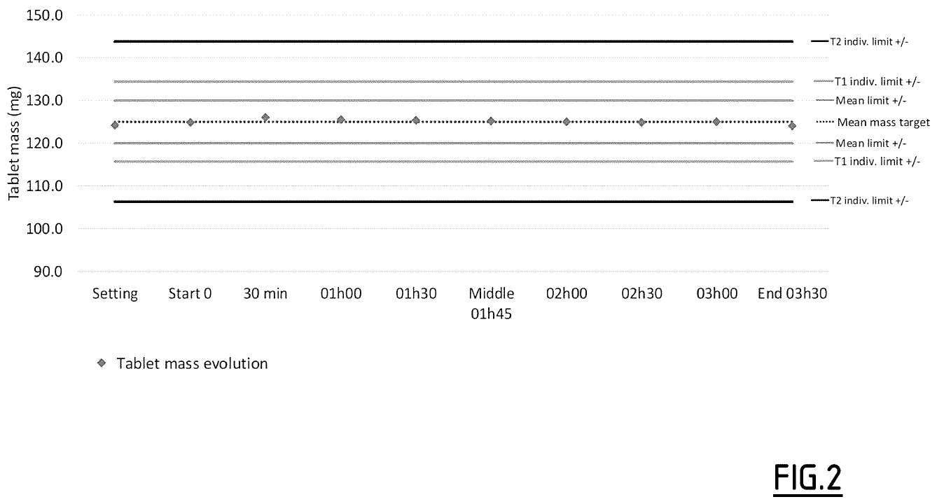

Mass evolution during compression was tested, the results of which are provided in FIG. 2. Tablet mass is very stable, and remains below 1.5%. Increase in RSD over time was attributed to lack of sufficient powder in the feeding system.

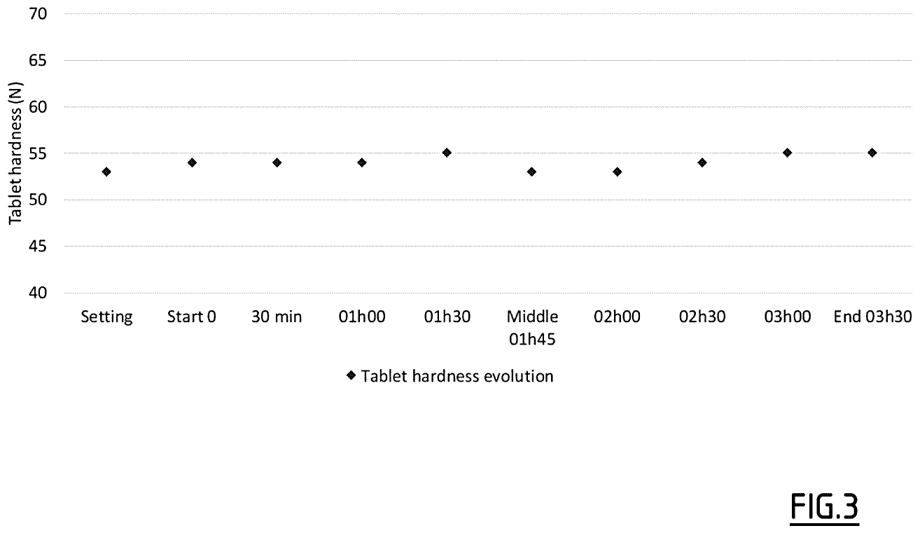

Tablet hardness was also evaluated during compression, and the results are provided in FIG. 3. Tablet hardness was as stable as the tablet mass.

The Batch 1 core tablets complied in terms of disintegration time and friability results. Tablet mass and hardness were very stable throughout the process.

### Example 2. Preparation of Exemplary Enteric-Coated Pitolisant (Batch 1)

The exemplary core tablet of Example 1 (Batch 1 core) was coated, first with an anti-moisture barrier coating, followed by a gastric resistant coating.

The coasting was performed in perforated pan using a spray nozzle of 0.8 mm. A first layer of OPADRY® amb II (3%) was performed (sub-layer). A coloring agent was also added in this layer. Then the gastroresistant coating was applied using ACRYL-EZE®. The plasticizers used were TEC (12%). The suspensions for the coatings were prepared using a magnetic bar and beaker to stir.

### Anti-Moisture Barrier Coating Step (Batch 1)

A suspension of OPADRY® amb II was prepared according to the manufacturing formulae provided in Table 6. Suspension preparation details are provided in Table 7.

TABLE 6
Manufacturing formulae for Amb II coating.

| | | Unit | Theoretical | Actual |
|---|---|---|---|---|
| | | formula | quantity | quantity |
| Raw material | Batch No. | (mg / unit) | (g) | (g) |
| Core | Batch 1 | 125 | 800 | 800 |
| OPADRY ® amb II | TKL53663 | 3.75 | 48 | 48 |
| clear | | | | |
| Top mill red | 9030806-L | 0.1 | 1.28 | 0.39497 |
| Water for injection | 19KK18GC | | 279.25 | 279.7¹ |
| Total | / | 128.85 | / | / |

¹15% solids, in excess (x2)

TABLE 7
Suspension preparation (OPADRY ® amb II)

| Step | Parameters | Batch 1 | |
|---|---|---|---|
| OPADRY ® amb II and top | Stirring speed | 450 to 500 | rpm |
| mill introduction | Duration | 45 | sec |
| Stirring | Stirring speed | 500 | rpm |

| | |
|---|---|
| Stirring time | 2 hours 30 mins |

Suspension was homogenous and stirring speed was decreased at 300 rpm for coating.

The coating parameters for the anti-moisture barrier layer (OPADRY® amb II) are provided in Table 8.

TABLE 8
Coating parameters for OPADRY ® amb II coating

| | | |
|---|---|---|
| Pre heat (min) | | 10 |
| Product temperature (° C.) | Start | 21.8 |
| | End | 45.6 |
| Pan speed (rpm) | Start | 5 (intermittent) |
| | End | 5 (intermittent) |
| Actual inlet air temp. (° C.) | Start | 65 |
| | End | 66 |
| Outlet air temp (° C.) | Start | 51.2 |
| | End | 54.6 |
| Air flow rate (m³/h) | Start | 300 |
| | End | 303 |
| Coating time (min) | | 80 |
| Product temp. (° C.) | Min | 45.6 |
| | Max | 47.4 |
| Pan speed (rpm) | Min | 25 |
| | Max | 28 |
| Actual inlet air temp. (° C.) | Min | 64.6 |
| | Max | 66.9 |
| Outlet air temp (° C.) | Min | 51.1 |
| | Max | 55.6 |
| Air flow rate (m³/h) | Min | 293 |
| | Max | 310 |
| Atomization pressure (bar) | | 1.4 to 1.2 |
| Spray width (bar) | | 1.8 to 1.6 |
| Spraying flow rate (g/min) | Min | 2.24 |
| | Max | 3.38 |
| Pump setting (%) | | 10-11 |
| Drying time (min) | | 5 |
| Product temp. (° C.) | Start | 47.4 |
| | End | 48.2 |
| Pan speed (rpm) | Start | 15 |
| | End | 15 |
| Actual inlet air temp. (° C.) | Start | 66.8 |
| | End | 66.5 |
| Outlet air temp (° C.) | Start | 52.7 |
| | End | 53.2 |
| Air flow rate (m³/h) | Start | 299 |
| | End | 311 |
| Weight gain (%) | | 3.0 |
| Cooling time (min) | | 20 |
| Product temp. (° C.) | Start | 48.2 |
| | End | 25.7 |
| Pan speed (rpm) | Start | 5 (intermittent) |
| | End | 5 (intermittent) |
| Actual inlet air temp. (° C.) | Start | 66.5 |
| | End | 33.5 |
| Outlet air temp (° C.) | Start | 53.2 |
| | End | 28.3 |
| Air flow rate (m³/h) | Start | 311 |
| | End | 305 |

### Gastroresistant Coating Step (Batch 1)

A suspension of ACRYL-EZE® was prepared according to the manufacturing formulae provided in Table 9. Suspension preparation details are provided in Table 10.

TABLE 9
Manufacturing formulae for ACRYL-EZE ® coating.

| | | Unit | Theoretical | Actual |
|---|---|---|---|---|
| | | formula | quantity | quantity |
| Raw material | Batch No. | (mg/unit) | (g) | (g) |
| OPADRY ® amb II | Batch 1 coated | 128.9375 | 767.7 | 767.7 |
| coated core | tablets (above) | | | |
| Acryl eze clear | TKL64755 | 19.34 | 240.0 | 240.0 |
| TEC | DTR527487 | 2.32 | 28.8 | 28.8 |
| Purified water | / | / | 960.0 | 960.0¹ |
| Total | / | 150.6 | / | / |

¹20% solids, in excess (×2)

TABLE 10
Suspension preparation (ACRYL-EZE ®)

| Step | Parameters | Batch 1 |
|---|---|---|
| PEG 8000 introduction | Stirring speed | 400 then 500 rpm |
| | Duration | 45 sec + 5 min |
| Acryl eze introduction | Stirring speed | About 600 rpm |
| | Duration | 1 min 30 sec + 5 min |
| Stirring | Stirring speed | About 500 tpm |
| | Stirring time | 2 hours 50 min |

Suspension was homogenous and stirring speed was decreased at 300 rpm for coating.

The coating parameters for the gastroresistant layer (ACRYL-EZE®) are provided in Table 11.

TABLE 11
Coating parameters for ACRYL-EZE ® coating

| | | |
|---|---|---|
| Pre heat (min) | | 6 |
| Product temperature (° C.) | Start | 21.0 |
| | End | 34.5 |
| Pan speed (rpm) | Start | 5 (intermittent) |
| | End | 5 (intermittent) |
| Actual inlet air temp. (° C.) | Start | 43.7 |
| | End | 44.0 |
| Outlet air temp (° C.) | Start | 38.9 |
| | End | 39.1 |
| Air flow rate (m³/h) | Start | 289 |
| | End | 289 |
| Coating time (min) | | 170 |
| Product temp. (° C.) | Min | 32.5 |
| | Max | 34.5 |
| Pan speed (rpm) | Min | 28 |
| | Max | 28 |
| Actual inlet air temp. (° C.) | Min | 42.7 |
| | Max | 44.7 |
| Outlet air temp (° C.) | Min | 36.5 |
| | Max | 39.1 |
| Air flow rate (m³/h) | Min | 289 |
| | Max | 303 |
| Atomization pressure (bar) | | 1.2 |
| Spray width (bar) | | 1.6 |
| Spraying flow rate (g/min) | Min | 2.60 |
| | Max | 5.11 |
| Pump setting (%) | | 10-13 |
| Drying time (min) | | 5 |
| Product temp. (° C.) | Start | 33.2 |
| | End | 36.6 |
| Pan speed (rpm) | Start | 15 |
| | End | 15 |
| Actual inlet air temp. (° C.) | Start | 44.7 |
| | End | 44.3 |
| Outlet air temp (° C.) | Start | 37.3 |
| | End | 37.9 |
| Air flow rate (m³/h) | Start | 296 |
| | End | 304 |
| Weight gain (%) | | 17.6 (corresponding |
| | | to 15.7% of GR) |
| Cooling time (min) | | 14 |
| Product temp. (° C.) | Start | 36.6 |
| | End | 25.5 |
| Pan speed (rpm) | Start | 5 (intermittent) |
| | End | 5 (intermittent) |
| Actual inlet air temp. (° C.) | Start | 44.3 |
| | End | 29.4 |
| Outlet air temp (° C.) | Start | 37.96 |
| | End | 26.3 |
| Air flow rate (m³/h) | Start | 296 |
| | End | 289 |

Final IPC was performed on the exemplary coated tablets. The results are provided in Table 12.

TABLE 12
IPC results of exemplary enteric-coated tablets

| | |
|---|---|
| Mean tablet mass (mg) | 149.0 |
| Min-Max mean tablet mass (mg) | 145.1-153.2 |
| CV (%) | 1.73 |
| Mean tablet hardness (N) | 110 |
| Min-Max mean hardness | 87-127 |
| CV (%) | 10.15 |
| Mean tablet thickness (mm) | 3.43 |
| Min-Max mean thickness | 3.40-3.46 |
| CV (%) | 0.60 |
| Mean tablet diameter (mm) | 7.78 |
| Min-Max mean diameter | 7.70-7.86 |
| CV (%) | 0.59 |

The addition of dye confirmed that the coating was uniform.

### Example 3. Preparation of Exemplary Enteric Coated Tablets (Batches 3-6)

Four additional batches were prepared, which are identified in Table 13. For each, the same core tablet prepared in Example 1 was used (Batch 1 core). Different coatings were applied following the protocols set forth below.

TABLE 13
Batches 3-6 comprising different amounts of gastroresistant
layer (GR), anti-moisture barrier layer (amb
II), and plasticizer (PEG or TEC).

| | F1 | F2 | F3 |
|---|---|---|---|
| Batch 3 | 15% weight gain GR | 3% weight gain ambII | PEG 8% |
| Batch 4 | 10% weight gain GR | 5% weight gain ambII | PEG 8% |
| Batch 5 | 10% weight gain GR | 3% weight gain ambII | TEC 12% |
| Batch 6 | 15% weight gain GR | 5% weight gain ambII | TEC 12% |

### Anti-Moisture Barrier Coating Step

A 3% suspension of OPADRY® amb II was prepared according to the manufacturing formulae provided in Table 14, and a 5% suspension of OPADRY® amb II was prepared according to the manufacturing formulae provided in Table 15. As both the OPADRY® amb II 3% and 5% coatings were made on the same day, only one solution was made for each. Suspension preparation details are provided in Table 16.

TABLE 14
Manufacturing formulae for 3% OPADRY ® amb II coating.

| | | Unit formula | Theoretical | Actual |
|---|---|---|---|---|
| Raw material | Batch No. | (mg/unit) | quantity (g) | quantity (g) |
| Core | Batch 1 core | 125 | 1600 | / |
| OPADRY ® | TKL53663 | 3.75 | 96 | 96 |
| amb II clear | | | | |
| Water for | 19KK18GC | | 544.0 | 544.1¹ |
| injection | | | | |
| total | / | 128.75 | / | / |

¹15% solids, in excess (×2)

TABLE 15
Manufacturing formulae for 5% OPADRY ® amb II coating.

| | | Unit formula | Theoretical | Actual |
|---|---|---|---|---|
| Raw material | Batch No. | (mg/unit) | quantity (g) | quantity (g) |
| Core | Batch 1 core | 125 | 1600 | / |
| OPADRY ® | TKL53663 | 6.25 | 160 | 160 |
| amb II clear | | | | |
| Purified water | 19KK18GC | | 906.67 | 906.7¹ |
| Total | / | 131.25 | / | / |

¹15% solids, in excess (×2)

TABLE 16
Suspension preparation (OPADRY ® amb II)

| Step | Parameters | Batches 3 and 5 | Batches 4 and 6 |
|---|---|---|---|
| AmbII | Stirring speed | 450 to 500 rpm | 300 rpm |
| introduction | Duration | 55 sec | 2 min 0 sec |
| Stirring | Stirring speed | 500 rpm | 300 rpm |
| | Stirring time | 1 hours 5 mins | 1 hour 30 min |

Suspension was homogenous and stirring speed was decreased at 300 rpm for coating.

The coating parameters for the anti-moisture barrier layer (OPADRY® amb II) applied to Batches 3-6 are provided in Table 17.

TABLE 17
Coating parameters for OPADRY ® amb II coating on Batches 3-6. Batch 1 is included for comparison

| | | Batch | | | | |
|---|---|---|---|---|---|---|
| | | 1 | 3 | 4 | 5 | 6 |
| Pre heat (min) | | 10 | 9 | 8 | 8 | 9 |
| Product | Start | 21.8 | 21.1 | 20.8 | 21.9 | 21.7 |
| temperature | End | 45.6 | 46.9 | 46.1 | 47.6 | 47.5 |
| (° C.) | | | | | | |
| Pan speed (rpm) | Start | 5 (intermittent) | 5 (intermittent) | 5 (intermittent) | 5 (intermittent) | 5 (intermittent) |
| | End | 5 (intermittent) | 5 (intermittent) | 5 (intermittent) | 5 (intermittent) | 5 (intermittent) |
| Actual inlet air | Start | 65 | 64.3 | 66.8 | 65.8 | 65.7 |
| temp. (° C.) | End | 66 | 65.5 | 65.1 | 66.2 | 65.9 |
| Outlet air temp | Start | 51.2 | 51.6 | 54.7 | 51.8 | 52.8 |
| (° C.) | End | 54.6 | 52.2 | 51.8 | 52.9 | 52.6 |
| Air flow rate | Start | 300 | 321 | 304 | 304 | 304 |
| (m³/h) | End | 303 | 305 | 303 | 306 | 300 |
| Coating time | | 80 | 70 | 100 | 80 | 110 |
| (min) | | | | | | |
| Product temp. | Min | 45.6 | 45.7 | 46.0 | 46.4 | 46.3 |
| (° C.) | Max | 47.4 | 47.1 | 47.3 | 47.6 | 47.5 |
| Pan speed (rpm) | Min | 25 | 25 | 25 | 25 | 25 |
| | Max | 28 | 28 | 28 | 28 | 25 |
| Actual inlet air | Min | 64.6 | 65.7 | 64.8 | 64.9 | 64.3 |
| temp. (° C.) | Max | 66.9 | 66.9 | 66.6 | 66.2 | 65.9 |
| Outlet air temp | Min | 51.1 | 51.5 | 51.5 | 51.5 | 51.4 |
| (° C.) | Max | 55.6 | 52.6 | 52.6 | 52.9 | 52.6 |
| Air flow rate | Min | 293 | 295 | 295 | 291 | 295 |
| (m³/h) | Max | 310 | 313 | 308 | 306 | 307 |
| Atomization | | 1.4 to 1.2 | 1.2 | 1.2 | 1.2 | 1.2 |
| pressure (bar) | | | | | | |
| Spray width | | 1.8 to 1.6 | 1.6 | 1.6 | 1.6 | 1.6 |
| (bar) | | | | | | |
| Spraying flow | Min | 2.24 | 2.58 | 2.68 | 2.75 | 2.66 |
| rate (g/min) | Max | 3.38 | 3.30 | 3.44 | 3.43 | 3.54 |
| Pump setting | | 10-11 | 10 | 10-11 | 10-11 | 10-11 |
| (%) | | | | | | |
| Drying time | | 5 | 5 | 5 | 5 | 5 |
| (min) | | | | | | |
| Product temp. | Start | 47.4 | 46.5 | 47.3 | 46.4 | 46.7 |
| (° C.) | End | 48.2 | 50.5 | 49.4 | 50.5 | 49.9 |
| Pan speed (rpm) | Start | 15 | 15 | 15 | 15 | 15 |
| | End | 15 | 15 | 15 | 15 | 15 |
| Actual inlet air | Start | 66.8 | 65.7 | 64.8 | 65.2 | 64.5 |
| temp. (° C.) | End | 66.5 | 66.3 | 65.2 | 65.0 | 64.3 |
| Outlet air temp | Start | 52.7 | 52.2 | 52.6 | 51.8 | 51.7 |
| (° C.) | End | 53.2 | 53.4 | 53.8 | 53.2 | 52.6 |
| Air flow rate | Start | 299 | 302 | 298 | 303 | 307 |
| (m³/h) | End | 311 | 303 | 298 | 295 | 306 |
| Weight gain | | 3.0 | 3.0 | 5.0 | 3.1 | 5.0 |
| (%) | | | | | | |
| Cooling time | | 20 | 15 | 14 | 14 | 18 |
| (min) | | | | | | |
| Product temp. | Start | 48.2 | 50.2 | 49.4 | 50.5 | 49.9 |
| (° C.) | End | 25.7 | 28.7 | 29.7 | 29.3 | 28.9 |
| Pan speed (rpm) | Start | 5 (intermittent) | 5 (intermittent) | 5 (intermittent) | 5 (intermittent) | 5 (intermittent) |
| | End | 5 (intermittent) | 5 (intermittent) | 5 (intermittent) | 5 (intermittent) | 5 (intermittent) |
| Actual inlet air | Start | 66.5 | 66.3 | 65.2 | 65.0 | 64.3 |
| temp. (° C.) | End | 33.5 | 36.5 | 37.2 | 37.6 | 35.9 |
| Outlet air temp | Start | 53.2 | 53.4 | 53.8 | 53.2 | 52.6 |
| (° C.) | End | 28.3 | 31.5 | 33.5 | 32.4 | 32.8 |
| Air flow rate | Start | 311 | 303 | 298 | 295 | 306 |
| (m³/h) | End | 305 | 303 | 303 | 296 | 295 |

### Gastroresistant Coating Step

Suspensions of ACRYL-EZE® were prepared for each of Batches 3-6 according to the manufacturing formulae provided in Tables 18-21. Suspension preparation details are provided in Table 22.

TABLE 18
Manufacturing formulae for ACRYL-EZE ® coating for Batch 3.

| | | Unit | Theoretical | Actual |
|---|---|---|---|---|
| | | formula | quantity | quantity |
| Raw material | Batch No. | (mg/unit) | (g) | (g) |
| amb II coated | Batch 3 coated | 128.75 | 800 | 784.1 |
| core | tablets (above) | | | |
| ACRYL-EZE ® | TKL64755 | 19.31 | 240 | 240 |
| clear | | | | |
| PEG8000 | DTR550141 | 1.545 | 19.2 | 19.2 |
| Purified water | / | / | 960.0 | 959.9¹ |
| Total | / | 149.61 | / | / |

¹20% solids, in excess (×2)

TABLE 19
Manufacturing formulae for ACRYL-EZE ® coating for Batch 4.

| | | Unit | Theoretical | Actual |
|---|---|---|---|---|
| | | formula | quantity | quantity |
| Raw material | Batch No. | (mg/unit) | (g) | (g) |
| amb II coated | Batch 4 coated | 131.25 | 800 | 763.6 |
| core | tablets (above) | | | |
| ACRYL-EZE ® | TKL64755 | 13.125 | 160 | 160 |
| clear | | | | |
| PEG8000 | DTR550141 | 1.05 | 12.8 | 12.8 |
| Purified water | / | / | 640 | 640.4¹ |
| Total | / | 145.425 | / | / |

¹20% solids, in excess (×2)

TABLE 20
Manufacturing formulae for ACRYL-EZE ® coating for Batch 5.

| | | Unit | Theoretical | Actual |
|---|---|---|---|---|
| | | formula | quantity | quantity |
| Raw material | Batch No. | (mg/unit) | (g) | (g) |
| amb II coated | Batch 5 coated | 128.75 | 800 | 765 |
| core | tablets (above) | | | |
| ACRYL-EZE ® | TKL64755 | 12.875 | 160 | 160 |
| clear | | | | |
| TEC | DTR527487 | 1.545 | 19.2 | 19.2 |
| Purified water | / | / | 640.0 | 640.1¹ |
| Total | / | 143.17 | / | / |

¹20% solids, in excess (×2)

TABLE 21
Manufacturing formulae for ACRYL-EZE ® coating for Batch 6.

| | | Unit | Theoretical | Actual |
|---|---|---|---|---|
| | | formula | quantity | quantity |
| Raw material | Batch No. | (mg/unit) | (g) | (g) |
| amb II coated | Batch 6 coated | 131.25 | 800 | 776.4 |
| core | tablets (above) | | | |
| ACRYL-EZE ® | TKL64755 | 19.6875 | 240 | 240 |
| clear | | | | |
| TEC | DTR527487 | 2.3625 | 28.8 | 28.8 |
| Purified water | / | / | 960 | 960.2¹ |
| Total | / | 153 | / | / |

¹20% solids, in excess (×2)

TABLE 22
Batches 3-6 suspension preparation (ACRYL-EZE ®)

| Step | Parameters | Batch 3 | Batch 4 | Batch 5 | Batch 6 |
|---|---|---|---|---|---|
| PEG 8000 | Stirring speed (rpm) | 500 | 300 | 350 | 350 |
| introduction | Duration | 40 sec + 5 min | 20 sec + 5 min | 5 min | 5 min |
| ACRYL-EZE ® | Stirring speed (rpm) | About 500 | 300 to 600 | 500 to 600 | 500 to 600 |
| introduction | Duration (min) | 2 min + 5 min | 2.5 min + 5 min | 1 min + 4 min | 2.25 min + 5 min |
| Stirring | Stirring speed (rpm) | About 400 | About 500 | About 500 | About 500 |
| | Stirring time | 3 h 0 min | 1 h 10 min | 1 h 0 min | 3 h 0 min |

Suspension was homogenous and stirring speed was decreased at 250/300 rpm for coating.

The coating parameters for the gastroresistant layer (ACRYL-EZE®) applied to Batches 3-6 are provided in Table 23.

TABLE 23
Coating parameters for ACRYL-EZE ® coating of Batches 3-6. Batch 1 is included for comparison

| | | Batch | | | | |
|---|---|---|---|---|---|---|
| | | 1 | 3 | 4 | 5 | 6 |
| Pre heat | | 6 | 7 | 8 | 8 | 7 |
| (min) | | | | | | |
| Product | Start | 21.0 | 20.9 | 20.4 | 20.9 | 20.5 |
| temperature | End | 34.5 | 37.3 | 34.6 | 35.1 | 34.7 |
| (° C.) | | | | | | |
| Pan speed | Start | 5 | 5 | 5 | 5 | 5 |
| (rpm) | | (intermittent) | (intermittent) | (intermittent) | (intermittent) | (intermittent) |
| | End | 5 | 5 | 5 | 5 | 5 |
| | | (intermittent) | (intermittent) | (intermittent) | (intermittent) | (intermittent) |
| Actual inlet | Start | 43.7 | 44.9 | 45.0 | 45.0 | 44.9 |
| air temp. | End | 44.0 | 45.1 | 45.0 | 44.1 | 44.4 |
| (° C.) | | | | | | |
| Outlet air | Start | 38.9 | 37.2 | 37.4 | 38.1 | 36.9 |
| temp (° C.) | End | 39.1 | 38.5 | 38.1 | 37.7 | 37.7 |
| Air flow | Start | 289 | 303 | 297 | 302 | 298 |
| rate (m³/h) | End | 289 | 295 | 301 | 301 | 310 |
| Coating | | 170 | 170 | 120 | 120 | 180 |
| time (min) | | | | | | |
| Product | Min | 32.5 | 32.1 | 32.3 | 32.3 | 32.0 |
| temp. (° C.) | Max | 34.5 | 37.3 | 34.6 | 35.1 | 34.7 |
| Pan speed | Min | 28 | 28 | 28 | 27 | 28 |
| (rpm) | Max | 28 | 28 | 28 | 28 | 28 |
| Actual inlet | Min | 42.7 | 43.7 | 44.3 | 44.1 | 44.3 |
| air temp. | Max | 44.7 | 45.2 | 45.0 | 44.7 | 45.3 |
| (° C.) | | | | | | |
| Outlet air | Min | 36.5 | 36.8 | 45.0 | 44.7 | 37.0 |
| temp (° C.) | Max | 39.1 | 38.5 | 36.9 | 36.8 | 37.8 |
| Air flow | Min | 289 | 294 | 293 | 296 | 296 |
| rate (m³/h) | Max | 303 | 308 | 303 | 307 | 310 |
| Atomization | | 1.2 | 1.2 | 1.2 | 1.2 | 1.2 |
| pressure (bar) | | | | | | |
| Spray width | | 1.6 | 1.6 | 1.6 | 1.6 | 1.6 |
| (bar) | | | | | | |
| Spraying | Min | 2.60 | 2.90 | 3.02 | 2.86 | 2.82 |
| flow rate | Max | 5.11 | 5.12 | 4.97 | 5.04 | 4.90 |
| (g/min) | | | | | | |
| Pump | | 10-13 | 9-15 | 10-14 | 10-14 | 10-15 |
| setting (%) | | | | | | |
| Drying time | | 5 | 5 | 5 | 5 | 5 |
| (min) | | | | | | |
| Product | Start | 33.2 | 33.1 | 33.5 | 32.5 | 32.7 |
| temp. (° C.) | End | 36.6 | 37.1 | 36.7 | 34.1 | 36.3 |
| Pan speed | Start | 15 | 15 | 15 | 15 | 15 |
| (rpm) | End | 15 | 15 | 15 | 15 | 15 |
| Actual inlet | Start | 44.7 | 44.5 | 44.5 | 44.5 | 44.5 |
| air temp. | End | 44.3 | 44.4 | 44.5 | 44.5 | 44.4 |
| (° C.) | | | | | | |
| Outlet air | Start | 37.3 | 37.0 | 37.0 | 37.1 | 37.2 |
| temp (° C.) | End | 37.9 | 39.1 | 38.1 | 38.3 | 38.8 |
| Air flow | Start | 296 | 306 | 303 | 297 | 300 |
| rate (m³/h) | End | 304 | 292 | 300 | 302 | 306 |
| Weight gain | | 17.6 | 16.1 | 11.0 | 11.3 | 17.3 |
| (%) | | (corresponding | corresponding | corresponding | corresponding | corresponding |
| | | to 15.7% of GR) | to 14.9% GR | to 9.8% GR | to 10.9% GR | to 15.4% GR |
| Cooling | | 14 | 9 | 13 | 8 | 8 |
| time (min) | | | | | | |
| Product | Start | 36.6 | 37.1 | 36.7 | 34.1 | 36.3 |
| temp. (° C.) | End | 25.5 | 29.8 | 27.5 | 27.9 | 28.7 |
| Pan speed | Start | 5 | 5 | 5 | 5 | 5 |
| (rpm) | | (intermittent) | (intermittent) | (intermittent) | (intermittent) | (intermittent) |
| | End | 5 | 5 | 5 | 5 | 5 |
| | | (intermittent) | (intermittent) | (intermittent) | (intermittent) | (intermittent) |
| Actual inlet | Start | 44.3 | 44.4 | 44.5 | 44.5 | 44.4 |
| air temp. | End | 29.4 | 37.4 | 32.0 | 33.3 | 34.9 |
| (° C.) | | | | | | |
| Outlet air | Start | 37.96 | 39.1 | 38.1 | 33.8 | 38.8 |
| temp (° C.) | End | 26.3 | 32.9 | 29.2 | 29.3 | 30.6 |
| Air flow | Start | 296 | 292 | 300 | 302 | 306 |
| rate (m³/h) | End | 289 | 299 | 301 | 297 | 308 |

Final IPC was performed on the exemplary tablets after anti-moisture barrier coating (OPADRY® amb II) was applied and after applying gastroresistant coating (ACRYL-EZE®) for Batches 3-6. The results are provided in Table 24.

TABLE 24
IPC results of exemplary enteric-coated tablets. Batch 1 is included for comparison.

| | | | | |
|---|---|---|---|---|
| Batch 1 | Batch 3 | Batch 4 | Batch 5 | Batch 6 |

OPADRY ® amb II-coated tablets

| | | | | | |
|---|---|---|---|---|---|
| Mean tablet mass (mg) | 128.2 | 127.5 | 129.2 | 127.8 | 129.3 |
| Min-Max mean tablet mass (mg) | 125.6-130.0 | 125.4-129.6 | 127.7-129.6 | 125.9-129.3 | 125.2-130.5 |
| CV (%) | 1.09 | 1.04 | 1.04 | 0.97 | 1.24 |
| Mean tablet hardness (N) | 74 | 73 | 80 | 75 | 79 |
| Min-Max mean hardness | 70-83 | 67-84 | 70-87 | 68-81 | 71-85 |
| CV (%) | 5.83 | 7.77 | 7.54 | 5.61 | 5.91 |
| Mean tablet thickness (mm) | 3.15 | 3.14 | 3.17 | 3.14 | 3.17 |
| Min-Max mean thickness | 3.11-3.16 | 3.11-3.17 | 3.14-3.19 | 3.12-3.17 | 3.15-3.20 |
| CV (%) | 0.48 | 0.72 | 0.57 | 0.52 | 0.54 |
| LOD (%) | 3.4 | 3.34 | 3.23 | 3.21 | 3.04 |
| Disintegration time in HCl | Min 4 m 1 s | Min 3 m 53 s | Min 4 m 22 s | Min 3 m 42 s | Min 4 m 21 s |
| (minutes (m) and seconds (s)). | Max 4 m 42 s | Max 5 m 49 s | Max 5 m 10 s | Max 4 m 37 s | Max 4 m 41 s |

ACRYL-EZE ® coated tablets

| | | | | | |
|---|---|---|---|---|---|
| Mean tablet mass (mg) | 149.0 | 147.8 | 143.5 | 141.6 | 152.2 |
| Min-Max mean tablet mass (mg) | 145.1-153.2 | 145.3-150.2 | 140.9-145.2 | 138.2-144.1 | 149.5-155.2 |
| CV (%) | 1.73 | 0.95 | 1.10 | 1.50 | 1.04 |
| Mean tablet hardness (N) | 110 | 110 | 92 | 91 | 115 |
| Min-Max mean hardness | 87-127 | 94-118 | 84-102 | 62-99 | 107-125 |
| CV (%) | 10.15 | 6.24 | 6.35 | 12.02 | 5.43 |
| Mean tablet thickness (mm) | 3.43 | 3.41 | 3.35 | 3.34 | 3.46 |
| Min-Max mean thickness | 3.40-3.46 | 3.38-3.45 | 3.30-3.39 | 3.30-3.37 | 3.42-3.48 |
| CV (%) | 0.60 | 0.65 | 0.96 | 0.65 | 0.60 |
| LOD (%) | 2.83 | 3.08 | 2.89 | 2.99 | 2.88 |

### Example 4. Preparation of an Exemplary Enteric Coated Tablets (Batch 7)

Another enteric-coated tablet batch (Batch 7) was prepared according to the following protocol.

This batch was prepared using the same core tablet prepared in Example 1 (Batch 1 core). The first coating was carried out with OPADRY® amb II BEIGE (reference 88A270004) (weight gain 3%) and the second coating was performed with ACRYL-EZE® clear at different percentages (up to 18%). The plasticizer used for this batch was PEG8000 (8%).

### Anti-Moisture Barrier Coating Step (Batch 7)

An OPADRY® amb II BEIGE suspension was prepared according to the manufacturing formulae provided in Table 25. Suspension preparation details are provided in Table 26.

TABLE 25
Manufacturing formulae for Batch 7 OPADRY ® amb
II BEIGE coating.

| | | Unit formula | Theoretical | Actual |
|---|---|---|---|---|
| Raw material | Batch No. | (mg/unit) | quantity (g) | quantity (g) |
| Core | Batch 1 core | 125 | 800.0 | 800.3 |
| ambII beige | TKL66169 | 3.75 | 48.0 | 48.0 |
| 88A270004 | | | | |
| Purified Water | / | / | 272 | 272.1¹ |
| Total | / | 128.75 | / | / |

¹15% solids, in excess (×2)

TABLE 26
Suspension preparation for Batch 7 OPADRY ® amb
II BEIGE coating.

| Step | Parameters | Batch 7 |
|---|---|---|
| AmbII | Stirring speed | About 500 rpm |
| introduction | Duration | 2 min 0 sec |
| Stirring | Stirring speed | About 600 rpm |
| | Stirring time | 55 min |

Suspension was homogenous and stirring speed was decreased at 300 rpm for coating.

The coating parameters for the anti-moisture barrier layer (OPADRY® amb II BEIGE) applied to Batch 7 is provided in Table 27.

TABLE 27
Coating parameters for OPADRY ® amb II coating
on Batch 7. Batch 1 is included for comparison.

| Batch | | 1 | 7 |
|---|---|---|---|
| Pre heat (min) | | 10 | 8 |
| Product | Start | 21.8 | 21.6 |
| temperature (° C.) | End | 45.6 | 47.4 |
| Pan speed (rpm) | Start | 5 (intermittent) | 4 (intermittent) |
| | End | 5 (intermittent) | 4 (intermittent) |
| Actual inlet air | Start | 65 | 64.8 |
| temp. (° C.) | End | 66 | 66.8 |
| Outlet air temp | Start | 51.2 | 52.1 |
| (° C.) | End | 54.6 | 53.6 |
| Air flow rate | Start | 300 | 304 |
| (m³/h) | End | 303 | 299 |
| Coating time | | 80 | 85 |
| (min) | | | |
| Product temp. | Min | 45.6 | 45.4 |
| (° C.) | Max | 47.4 | 47.4 |
| Pan speed (rpm) | Min | 25 | 28 |
| | Max | 28 | 28 |
| Actual inlet air | Min | 64.6 | 66.8 |
| temp. (° C.) | Max | 66.9 | 68.3 |
| Outlet air temp | Min | 51.1 | 52.0 |
| (° C.) | Max | 55.6 | 53.8 |
| Air flow rate | Min | 293 | 295 |
| (m³/h) | Max | 310 | 307 |
| Atomization | | 1.4 to 1.2 | 1.2 |
| pressure (bar) | | | |
| Spray width (bar) | | 1.8 to 1.6 | 1.6 |
| Spraying flow | Min | 2.24 | 2.54 |
| rate (g/min) | Max | 3.38 | 3.10 |
| Pump setting (%) | | 10-11 | 10 |
| Drying time (min) | | 5 | 5 |
| Product temp. | Start | 47.4 | 47.0 |
| (° C.) | End | 48.2 | 49.5 |
| Pan speed (rpm) | Start | 15 | 15 |
| | End | 15 | 15 |
| Actual inlet air | Start | 66.8 | 66.7 |
| temp. (° C.) | End | 66.5 | 67.7 |
| Outlet air temp | Start | 52.7 | 53.6 |
| (° C.) | End | 53.2 | 53.8 |
| Air flow rate | Start | 299 | 302 |
| (m³/h) | End | 311 | 299 |
| Weight gain (%) | | 3.0 | 2.9 |
| Cooling time | | 20 | 22 |
| (min) | | | |
| Product temp. | Start | 48.2 | 47.0 |
| (° C.) | End | 25.7 | 29 |
| Pan speed (rpm) | Start | 5 (intermittent) | 5 (intermittent) |
| | End | 5 (intermittent) | 5 (intermittent) |
| Actual inlet air | Start | 66.5 | 66.7 |
| temp. (° C.) | End | 33.5 | 37.5 |
| Outlet air temp | Start | 53.2 | 53.6 |
| (° C.) | End | 28.3 | 31.4 |
| Air flow rate | Start | 311 | 299 |
| (m³/h) | End | 305 | 299 |

### Gastroresistant Coating Step (Batch 7)

Suspensions of ACRYL-EZE® were prepared for Batch 7 according to the manufacturing formulae provided in Table 28. Suspension preparation details are provided in Table 29.

TABLE 28
Manufacturing formulae for acryl-ease coating for Batch 7.

| | | Unit | | Actual |
|---|---|---|---|---|
| | | formula | Theoretical | quantity |
| Raw material | Batch No. | (mg/unit) | quantity (g) | (g) |
| amb II | Batch 7 coated | 128.75 | 800 | 787.5 |
| coated core | tablets (above) | | | |
| ACRYL-EZE ® | ME-22-083 | 21.8875 | 272 | 272 |
| clear | | | | |
| PEG8000 | DTR550141 | 1.751 | 21.76 | 21.8 |
| Purified water | / | / | 1088.0 | 1087.9¹ |
| Total | / | 152.3885 | / | / |

¹20% solids, in excess (×2)

TABLE 29
Suspension preparation details for Batch 7.

| Step | Parameters | Batch 7 |
|---|---|---|
| PEG 8000 | Stirring speed (rpm) | About 600 |
| introduction | Duration | 30 sec + 7 min |
| ACRYL-EZE ® | Stirring speed (rpm) | About 600 to 700 |
| introduction | Duration (min) | 3 min |
| Stirring | Stirring speed (rpm) | About 500 |
| | Stirring time | 2 h 30 min |

The coating parameters for the gastroresistant layer (ACRYL-EZE®) applied to Batch 7 is provided in Table 30.

TABLE 30
Coating parameters for ACRYL-EZE ® coating
of Batch 7. Batch 1 is included for comparison

| Batch | | 1 | 7 |
|---|---|---|---|
| Pre heat (min) | | 6 | 5 |
| Product | Start | 21.0 | 26.6 |
| temperature (° C.) | End | 34.5 | 34.9 |
| Pan speed (rpm) | Start | 5 (intermittent) | 5 (intermittent) |
| | End | 5 (intermittent) | 5 (intermittent) |
| Actual inlet air | Start | 43.7 | 44.8 |
| temp. (° C.) | End | 44.0 | 44.3 |
| Outlet air temp | Start | 38.9 | 36.8 |
| (° C.) | End | 39.1 | 38.0 |
| Air flow rate | Start | 289 | 300 |
| (m³/h) | End | 289 | 300 |
| Coating time | | 170 | 190 |
| (min) | | | |
| Product temp. | Min | 32.5 | 31.9 |
| (° C.) | Max | 34.5 | 34.9 |
| Pan speed (rpm) | Min | 28 | 28 |
| | Max | 28 | 28 |
| Actual inlet air | Min | 42.7 | 42.4 |
| temp. (° C.) | Max | 44.7 | 44.3 |
| Outlet air temp | Min | 36.5 | 36.2 |
| (° C.) | Max | 39.1 | 38.0 |
| Air flow rate | Min | 289 | 295 |
| (m³/h) | Max | 303 | 304 |
| Atomization | | 1.2 | 1.2 |
| pressure (bar) | | | |
| Spray width (bar) | | 1.6 | 1.6 |
| Spraying flow | Min | 2.60 | 2.70 |
| rate (g/min) | Max | 5.11 | 4.95 |
| Pump setting (%) | | 10-13 | 10-15 |
| Drying time (min) | | 5 | 5 |
| Product temp. | Start | 33.2 | 32.7 |
| (° C.) | End | 36.6 | 35.8 |
| Pan speed (rpm) | Start | 15 | 15 |
| | End | 15 | 15 |
| Actual inlet air | Start | 44.7 | 42.4 |
| temp. (° C.) | End | 44.3 | 42.4 |
| Outlet air temp | Start | 37.3 | 36.2 |
| (° C.) | End | 37.9 | 37.6 |
| Air flow rate | Start | 296 | 304 |
| (m³/h) | End | 304 | 299 |
| Weight gain (%) | | 17.6 (corresponding | 18.8 (corresponding |
| | | to 15.7% of GR) | to 16.9% of GR) |
| Cooling time | | 14 | 8 |
| (min) | | | |
| Product temp. | Start | 36.6 | 35.8 |
| (° C.) | End | 25.5 | 28.3 |
| Pan speed (rpm) | Start | 5 (intermittent) | 5 (intermittent) |
| | End | 5 (intermittent) | 5 (intermittent) |
| Actual inlet air | Start | 44.3 | 42.4 |
| temp. (° C.) | End | 29.4 | 36.6 |
| Outlet air temp | Start | 37.96 | 37.6 |
| (° C.) | End | 26.3 | 30.0 |
| Air flow rate | Start | 296 | 299 |
| (m³/h) | End | 289 | 303 |

### Example 5. Quality Control Testing

Batches 1 and 3-6 were subjected to quality control testing, including disintegration, dissolution, content uniformity. Batches 3, 5, and 6 were also subjected to stability testing for appearance, average mass, water content, assay and purity.

### Disintegration-Batch 1

Disintegration studies were performed on n=6 tablets from a representative sample of Batch 1 with 10% GR (Batch 1-1); 12% GR (Batch 1-2); and 15% GR coating (Batch 1-3). Studies were performed in two steps, first in HCl 0.1M for 2 hours (without disks), then in phosphate buffer at pH 6.8 for 1 hour (with disks), according to the Ph.Eur. 2.9.1/USP <711>.

Results of the studies are provided in Table 31. All disintegration results were compliant.

TABLE 31
Disintegration results for Batches 1-1, 1-2, and 1-3

| | | Batch 1-1 | Batch 1-2 | Batch 1-3 |
|---|---|---|---|---|
| | Specification | 10% GR | 12% GR | 15% GR |
| First in HCl | No tablet shows | comply | comply | Comply |
| 0.1M for 2 h | signs of either | | | |
| (without disks) | disintegration | | | |
| | (apart from | | | |
| | fragments of | | | |
| | coating) or | | | |
| | cracks that | | | |
| | would allow | | | |
| | the escape | | | |
| | of contents | | | |
| Then phosphate | Comply with | 7 min | 9 min | 9 min |
| buffer pH 6.8 for | Eur. Ph. | | | |
| 1 h (with disks) | 2.9.1 <1 hour | | | |

### Disintegration—Batches 3-6

Disintegration studies were performed on n=6 tablets from a representative sample of Batches 3-6, in two steps, first in HCl 0.1M for 2 hours (without disks), then in phosphate buffer pH 6.8 for 1 hour (with disks), according to the Ph.Eur. 2.9.1/USP <711>. For the Batch 4 disintegration results obtained were not compliant, in acid media, 5 units were open after 120 minutes in HCl 0.1M. For Batch 6, 2 tablets were swollen after 120 minutes in HCl 0.1M. The other batches 3 and 5 were compliant. Results are provided in Table 32

TABLE 32
Disintegration Studies with Batches 3-6

| | Specification | Batch 3 | Batch 4 | Batch 5 | Batch 6 |
|---|---|---|---|---|---|
| First in HCl | No tablet shows | Comply | Do not | Comply | Do not |
| 0.1M for 2 h | signs of either | | comply | | comply |
| (without disks) | disintegration (apart | | (5 tablets | | (2 swollen) |
| | from fragments of | | open) | | |
| | coating) or cracks | | | | |
| | that would allow the | | | | |
| | escape of contents | | | | |
| Then phosphate | Comply with | 8 min | 7 min | 4 min | 8 min |
| buffer pH 6.8 for | Eur. Ph. 2.9.1 | | | | |
| 1 h (with disks) | | | | | |

### Disintegration—Batch 7

Disintegration studies were performed on n=6 tablets from a representative sample of Batch 7 with 10% GR (7-1), 12% GR (7-2), 15% GR (7-3), and 17% GR (7-4). Studies were performed in two steps, first in HCl 0.1M for 2 hours (without disks), then in phosphate buffer pH 6.8 for 1 hour (with disk), according to the Ph.Eur. 2.9.1/USP <711>.

For all the amount tested of GR coating (from 10% to 17%) of the Batch 7 disintegration results obtained are compliant, in acid media and in phosphate buffer. Results are provided in Table 33.

TABLE 33
Disintegration results for Batches 7-1, 7-2, 7-3, and 7-4

| | Specification | Batch 3 | Batch 4 | Batch 5 | Batch 6 |
|---|---|---|---|---|---|
| First in HCl | No tablet shows | Comply | Comply | Comply | Comply |
| 0.1M for | signs of either | | | | |
| 2 h (without | disintegration | | | | |
| disks) | (apart from | | | | |
| | fragments of | | | | |
| | coating) or | | | | |
| | cracks that | | | | |
| | would allow | | | | |
| | the escape | | | | |
| | of contents | | | | |
| Then | Comply with | 9 min | 10 min | 13 min | 12 min |
| phosphate | Eur. Ph. 2.9.1 | | | | |
| buffer pH 6.8 | | | | | |
| for 1 h | | | | | |
| (with disks) | | | | | |

### Dissolution—Batch 1

Dissolution testing was performed on gastroresistant film coated tablets from a representative sample (n=6 tablets). Results are provided in Tables 34-36, which show a plateau was reached at 100% dissolution and a low variability was observed between each unit. The dissolution results comply with specification at level 1. There are no significant differences in dissolution profiles regardless of the amount of gastroresistant coating (GR) by comparing 10% GR (Batch 1-1); 12% GR (Batch 1-2); and 15% GR coating (Batch 1-3).

TABLE 34
Dissolution Results for Batch 1-1
Dissolution in HCl 0.1N/Ph 6.8 (75/25) batch 1-1 (10% GR), 20 mg pitolisant hydrochloride

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0 | 0 | 0 | 0 | 0 | 0 | 0 | 0 | 0 | 0 | 0 |
| 120 | 0 | 0 | 0 | 0 | 0 | 0 | 0 | 0 | 0 | 0 | 0 |
| 125 | 0 | 0 | 0 | 0 | 0 | 0 | 0 | 0 | 0 | 0 | 0 |
| 130 | 4.7 | 80.4 | 77.9 | 75.4 | 79.3 | 82.9 | 67 | 30.5 | 45.66 | 5 | 83 |
| 135 | 92.8 | 96.4 | 93.5 | 94.4 | 97.3 | 94.7 | 95 | 1.7 | 1.78 | 93 | 97 |
| 140 | 98.8 | 99 | 97.7 | 97.0 | 100.2 | 97.2 | 98 | 1.2 | 1.24 | 97 | 100 |
| 150 | 99.3 | 99 | 98.0 | 97.4 | 100.5 | 97.2 | 99 | 1.3 | 1.28 | 97 | 100 |
| 165 | 99.3 | 99.4 | 98.2 | 97.6 | 100.6 | 97.4 | 99 | 1.2 | 1.26 | 97 | 101 |
| 180 | 99.5 | 99.7 | 98.4 | 97.4 | 100.9 | 97.3 | 99 | 1.4 | 1.41 | 97 | 101 |
| 195 | 99.3 | 99.7 | 98.5 | 97.5 | 100.8 | 97.6 | 99 | 1.3 | 1.31 | 97 | 101 |

TABLE 35
Dissolution Results for Batch 1-2
Dissolution in HCl 0.1N/Ph 6.8 (75/25) batch 1-2 (12% GR), 20 mg pitolisant hydrochloride

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0.6 | 0.2 | 0.3 | 0.2 | 0.2 | 1.6 | 1 | 0.6 | 103.79 | 0 | 2 |
| 120 | 0 | 0 | 1.0 | 0 | 0 | 0.5 | 0 | 0.4 | 169.65 | 0 | 1 |
| 125 | 0.9 | 0.6 | 0.5 | 0.5 | 1.3 | 0.4 | 1 | 0.4 | 50.37 | 0 | 1 |
| 130 | 0 | 0.3 | 27.3 | 0.8 | 0.8 | 0.6 | 5 | 10.9 | 220.90 | 0 | 27 |
| 135 | 91.4 | 90.5 | 92.6 | 84.9 | 91.3 | 87.3 | 90 | 2.9 | 3.25 | 85 | 93 |
| 140 | 96.3 | 97.0 | 98.2 | 93.8 | 96.3 | 92.6 | 96 | 2.1 | 2.19 | 93 | 98 |
| 150 | 97.6 | 98.6 | 99.2 | 96.6 | 98.1 | 96.5 | 98 | 1.1 | 1.11 | 97 | 99 |
| 165 | 97.6 | 98.2 | 99.8 | 96.7 | 98.6 | 97.0 | 98 | 1.1 | 1.17 | 97 | 100 |
| 180 | 97.8 | 98.5 | 99.7 | 97.1 | 98.8 | 96.5 | 98 | 1.2 | 1.20 | 97 | 100 |
| 195 | 97.8 | 98.6 | 99.7 | 98.0 | 99.1 | 96.6 | 98 | 1.1 | 1.13 | 97 | 100 |

TABLE 36
Dissolution Results for Batch 1-3
Dissolution in HCl 0.1N/Ph 6.8 (75/25) batch 1-2 (12% GR), 20 mg pitolisant hydrochloride

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0 | 0.3 | 0.5 | 0 | 1.1 | 0.6 | 0 | 0.4 | 99.33 | 0 | 1 |
| 120 | 0.5 | 0 | 0 | 0.3 | 0.7 | 0 | 0 | 0.3 | 122.24 | 0 | 1 |
| 125 | 0.5 | 0 | 0 | 0.6 | 0 | 0.9 | 0 | 0.4 | 116.60 | 0 | 1 |
| 130 | 7.6 | 57.8 | 4.9 | 3.8 | 10.9 | 0.8 | 14 | 21.6 | 150.76 | 1 | 58 |
| 135 | 91.8 | 91 | 87.5 | 94.2 | 90.7 | 93.1 | 91 | 2.3 | 2.53 | 87 | 94 |
| 140 | 95.6 | 95.4 | 93.5 | 98.9 | 96.6 | 96.8 | 96 | 1.8 | 1.85 | 94 | 99 |
| 150 | 97 | 97.1 | 96.7 | 99.9 | 98.0 | 99.4 | 98 | 1.4 | 1.41 | 97 | 100 |
| 165 | 97.6 | 97.7 | 96.3 | 101.2 | 98.3 | 100.5 | 99 | 1.9 | 1.90 | 96 | 101 |
| 180 | 97.5 | 97.5 | 96.8 | 101.5 | 99.0 | 100.9 | 99 | 2.0 | 1.99 | 97 | 101 |
| 195 | 98 | 98.4 | 97.1 | 101.7 | 98.8 | 101.9 | 99 | 2.04 | 2.04 | 97 | 102 |

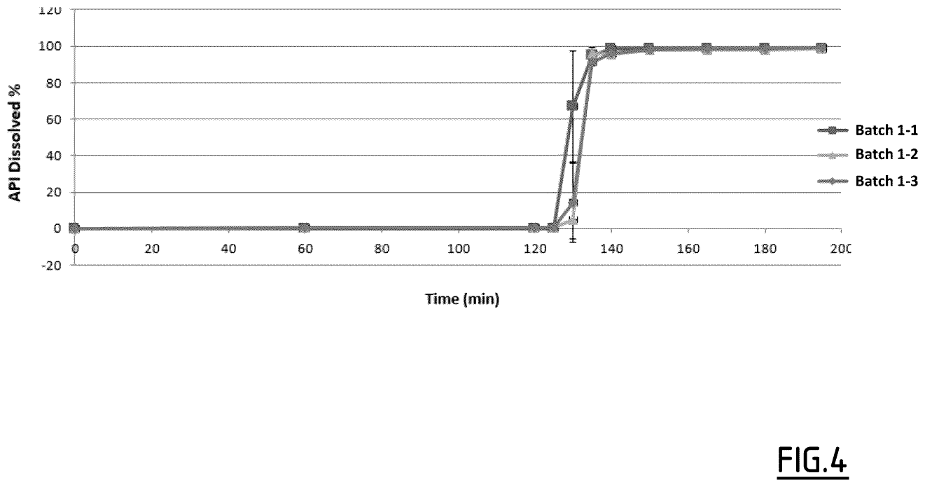

An overlay of dissolution profiles for Batches 1-1 to 1-3 is provided in FIG. 4.

### Dissolution—Batches 3-6

Dissolution testing was performed on gastroresistant film coated tablets from a representative sample (n=6 tablets) from batches 3-6. Results are provided in Tables 37-40. The dissolution results comply with the specifications except for Batch 4, all units exceed 10% dissolved after 120 minutes.

TABLE 37
Dissolution Results for Batch 3
Dissolution in HCl 0.1N/Ph 6.8 (75/25) Batch 3, 20 mg pitolisant hydrochloride

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0 | 0 | 0 | 0 | 0 | 0.1 | 0 | 0.1 | 244.95 | 0 | 0 |
| 120 | 0.2 | 0.1 | 0 | 0 | 0 | 0.0 | 0 | 0.1 | 168.99 | 0 | 0 |
| 125 | 2.3 | 0 | 0 | 0 | 0 | 0.0 | 0 | 1.0 | 244.95 | 0 | 2 |
| 130 | 18.7 | 20.6 | 0 | 0.8 | 67.9 | 20.1 | 21 | 24.7 | 115.67 | 0 | 68 |
| 135 | 87.3 | 87.3 | 85.8 | 90.6 | 90.3 | 90.6 | 89 | 2.1 | 2.36 | 86 | 91 |
| 140 | 93.7 | 96.1 | 94.5 | 97.4 | 97.2 | 96.8 | 96 | 1.5 | 1.58 | 94 | 97 |
| 150 | 96.5 | 100.4 | 97.2 | 99.8 | 100.7 | 99.3 | 99 | 1.7 | 1.76 | 96 | 101 |
| 165 | 97.8 | 100.9 | 98.2 | 99.3 | 101.0 | 99.8 | 99 | 1.4 | 1.36 | 98 | 101 |
| 180 | 97.5 | 101.1 | 98.1 | 98.9 | 101.3 | 99.4 | 99 | 1.5 | 1.55 | 98 | 101 |
| 195 | 97.8 | 101.0 | 97.8 | 100.3 | 102.6 | 100.0 | 100 | 1.9 | 1.88 | 98 | 103 |

TABLE 38
Dissolution Results for Batch 4
Dissolution in HCl 0.1N/Ph 6.8 (75/25) Batch 4, 20 mg pitolisant hydrochloride

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 77.6 | 10.5 | | 0.4 | 103.2 | 7.5 | 40 | 47.2 | 118.46 | 0 | 103 |
| 120 | 90.6 | 46.1 | | 28.8 | 105.9 | 49.5 | 64 | 32.5 | 50.69 | 29 | 106 |
| 125 | 86.6 | 46.6 | | 30.1 | 99.4 | 49.0 | 62 | 29.3 | 46.98 | 30 | 99 |
| 130 | 88.3 | 49.5 | | 71.5 | 100.5 | 52.6 | 72 | 22.1 | 30.56 | 49 | 100 |
| 135 | 91.6 | 55.3 | | 89.3 | 101.5 | 81.7 | 84 | 17.4 | 20.81 | 55 | 101 |
| 140 | 96.5 | 88.0 | | 96.4 | 101.4 | 93.3 | 95 | 4.9 | 5.20 | 88 | 101 |
| 150 | 97.8 | 99.4 | | 98.6 | 101.6 | 98.4 | 99 | 1.5 | 1.52 | 98 | 102 |
| 165 | 98.0 | 100.6 | | 98.9 | 101.4 | 98.9 | 100 | 1.4 | 1.38 | 98 | 101 |
| 180 | 97.2 | 100.3 | | 98.6 | 101.5 | 98.9 | 99 | 1.6 | 1.65 | 97 | 101 |
| 195 | 97.9 | 100.4 | | 98.9 | 101.1 | 99.1 | 99 | 1.3 | 1.28 | 98 | 101 |

TABLE 39
Dissolution Results for Batch 5
Dissolution in HCl 0.1N/Ph 6.8 (75/25) Batch 5, 20 mg pitolisant hydrochloride

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0 | 0 | 0 | 0 | 0 | 0 | 0 | 0 | 0 | 0 | 0 |
| 120 | 2.6 | 5.3 | 0 | 2.7 | 4.9 | 2.9 | 3 | 1.9 | 62.37 | 0 | 5 |
| 125 | 3.0 | 5.7 | 0.2 | 3.2 | 5.6 | 3.5 | 4 | 2.0 | 57.29 | 0 | 6 |
| 130 | 5.7 | 8.3 | 86.4 | 5.5 | 8.4 | 6.3 | 20 | 32.4 | 159.98 | 6 | 86 |
| 135 | 57.3 | 82.2 | 96.0 | 51.0 | 79.1 | 86.8 | 75 | 17.5 | 23.24 | 51 | 96 |
| 140 | 99.2 | 94.4 | 99.1 | 93.9 | 99.8 | 97.3 | 97 | 2.6 | 2.64 | 94 | 100 |
| 150 | 101.4 | 95.3 | 99.5 | 97.3 | 101.1 | 98.5 | 99 | 2.3 | 2.37 | 95 | 101 |
| 165 | 101.7 | 95.7 | 99.9 | 97.8 | 100.9 | 98.6 | 99 | 2.2 | 2.23 | 96 | 102 |
| 180 | 102.3 | 95.5 | 99.9 | 97.6 | 101.3 | 98.3 | 99 | 2.5 | 2.53 | 95 | 102 |
| 195 | 101.8 | 95.6 | 99.8 | 98.1 | 101.3 | 98.7 | 99 | 2.3 | 2.30 | 96 | 102 |

TABLE 40
Dissolution Results for Batch 6
Dissolution in HCl 0.1N/Ph 6.8 (75/25) Batch 6, 20 mg pitolisant hydrochloride

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0 | 0 | 0 | 0 | 0 | 0 | 0 | 0 | 0 | 0 | 0 |
| 120 | 1.2 | 0 | 0.5 | 0 | 0 | 0 | 0 | 0.5 | 172.88 | 0 | 1 |
| 125 | 1.3 | 0 | 0.7 | 0.6 | 0.3 | 0.3 | 1 | 0.5 | 88.48 | 0 | 1 |
| 130 | 2.0 | 6.0 | 1.7 | 2.0 | 1.0 | 1.0 | 2 | 1.9 | 82.50 | 1 | 6 |
| 135 | 8.9 | 57.4 | 8.3 | 35.8 | 38.4 | 38.4 | 30 | 18.8 | 62.85 | 8 | 57 |
| 140 | 80.1 | 87.8 | 82.3 | 89.4 | 94.2 | 94.2 | 87 | 5.2 | 5.97 | 80 | 94 |
| 150 | 98.1 | 99.1 | 97.3 | 97.6 | 100.9 | 100.9 | 99 | 1.3 | 1.34 | 97 | 101 |
| 165 | 99.2 | 100.2 | 98.8 | 98.5 | 101.2 | 101.2 | 100 | 1.0 | 1.00 | 99 | 101 |
| 180 | 99.3 | 100.0 | 99.1 | 98.7 | 101.3 | 101.3 | 100 | 0.9 | 0.90 | 99 | 101 |
| 195 | 99.7 | 100.4 | 99.2 | 98.8 | 101.7 | 101.7 | 100 | 1.0 | 1.02 | 99 | 102 |

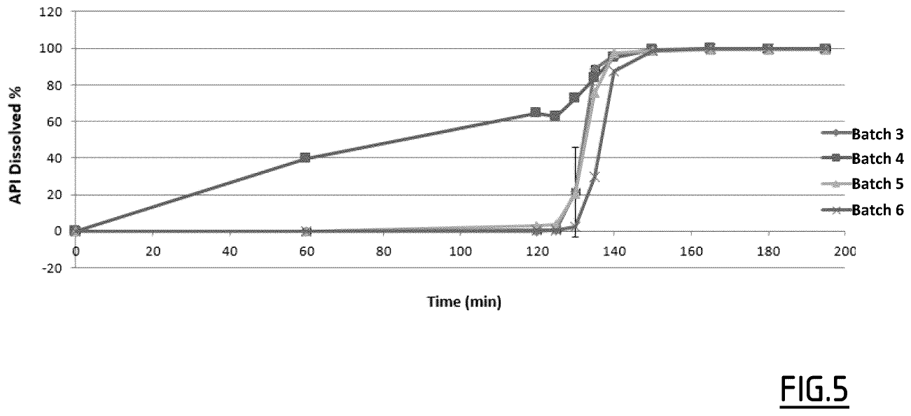

An overlay of dissolution profiles for Batches 4-6 is provided in FIG. 5.

### Dissolution—Batch 7

The dissolution testing was performed on Gastro resistant film coated tablets from a representative sample (n=6) of Batch 7 tablets with 10% GR (7-1), 12% GR (7-2), 15% GR (7-3), and 17% GR (7-4). The dissolution results comply with the specifications except for the batch 7-1 (10% GR coating). Results are provided in Tables 41-44.

TABLE 41
Dissolution results for Batch 7-1 (10% GR coating)
Batch 7-1 10% pelliculage GR

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0.0 | 0 | | |
| 60 | 0.330 | 1.397 | 0.359 | 0.362 | 0.503 | 1.496 | 1 | 0.6 | 74.28 | 0 | 1 |
| 120 | 0.000 | 35.865 | 0.108 | 21.997 | 6.320 | 32.163 | 16 | 16.1 | 100.08 | 0 | 36 |
| 125 | 0.248 | 42.809 | 0.000 | 23.496 | 6.459 | 57.651 | 22 | 24.1 | 110.68 | 0 | 58 |
| 130 | 11.676 | 78.021 | 52.928 | 29.655 | 10.614 | 70.140 | 42 | 29.2 | 69.26 | 11 | 78 |
| 135 | 88.527 | 86.510 | 90.772 | 42.745 | 53.726 | 80.194 | 74 | 20.4 | 27.62 | 43 | 91 |
| 140 | 100.873 | 97.151 | 96.916 | 57.966 | 93.254 | 98.274 | 91 | 16.2 | 17.90 | 58 | 101 |
| 150 | 102.990 | 99.623 | 100.209 | 93.617 | 98.762 | 102.192 | 100 | 3.3 | 3.33 | 94 | 103 |
| 165 | 102.897 | 99.677 | 100.068 | 96.520 | 99.232 | 102.381 | 100 | 2.3 | 2.31 | 97 | 103 |
| 180 | 101.878 | 98.199 | 99.756 | 96.967 | 98.799 | 102.268 | 100 | 2.1 | 2.10 | 97 | 102 |
| 195 | 102.306 | 99.427 | 99.728 | 97.316 | 99.013 | 101.862 | 100 | 1.9 | 1.86 | 97 | 102 |

TABLE 42
Dissolution results for Batch 7-2 (13% GR coating)
Batch 7-2 13% pelliculage GR

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0.0 | 0 | | |
| 60 | 0.683 | 0.317 | 0.547 | 0.396 | 0.450 | 0.303 | 0 | 0.1 | 32.39 | 0 | 1 |
| 120 | 0.333 | 0.291 | 0.385 | 0.139 | 0.157 | 0.091 | 0 | 0.1 | 51.30 | 0 | 0 |
| 125 | 0.000 | 0.439 | 0.170 | 0.404 | 0.000 | 0.282 | 0 | 0.2 | 89.09 | 0 | 0 |
| 130 | 0.482 | 28.818 | 0.000 | 0.000 | 0.730 | 85.997 | 19 | 34.6 | 178.88 | 0 | 86 |
| 135 | 93.276 | 87.871 | 87.429 | 87.422 | 85.624 | 94.409 | 89 | 3.6 | 4.02 | 86 | 94 |
| 140 | 95.002 | 100.846 | 92.925 | 93.319 | 96.436 | 97.447 | 96 | 2.9 | 3.07 | 93 | 101 |
| 150 | 99.618 | 104.687 | 98.686 | 97.171 | 102.489 | 99.831 | 100 | 2.7 | 2.71 | 97 | 105 |
| 165 | 100.443 | 105.159 | 99.735 | 97.691 | 102.468 | 100.148 | 101 | 2.6 | 2.55 | 98 | 105 |
| 180 | 100.127 | 104.752 | 100.011 | 97.254 | 102.248 | 99.776 | 101 | 2.5 | 2.53 | 97 | 105 |
| 195 | 99.708 | 104.671 | 99.437 | 97.566 | 102.860 | 99.500 | 101 | 2.6 | 2.60 | 98 | 105 |

TABLE 43
Dissolution results for Batch 7-3 (15% GR coating)
Batch 7-3 15% coating GR

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0.0 | 0 | | |
| 60 | 0.230 | 0.234 | 0.236 | 0.214 | 0.285 | 0.381 | 0 | 0.1 | 23.70 | 0 | 0 |
| 120 | 0.116 | 0.252 | 0.114 | 0.112 | 0.176 | 0.142 | 0 | 0.1 | 36.05 | 0 | 0 |
| 125 | 0.000 | 0.000 | 0.000 | 0.000 | 0.214 | 0.119 | 0 | 0.1 | 164.10 | 0 | 0 |
| 130 | 72.705 | 34.035 | 0.000 | 0.000 | 0.141 | 0.000 | 18 | 30.1 | 169.17 | 0 | 73 |
| 135 | 90.468 | 71.036 | 83.882 | 90.381 | 88.095 | 85.255 | 85 | 7.3 | 8.57 | 71 | 90 |
| 140 | 93.913 | 87.777 | 82.354 | 97.968 | 96.927 | 93.516 | 92 | 5.9 | 6.46 | 82 | 98 |
| 150 | 99.414 | 100.288 | 97.496 | 101.513 | 99.777 | 99.613 | 100 | 1.3 | 1.31 | 97 | 102 |
| 165 | 99.630 | 101.014 | 97.887 | 101.411 | 100.193 | 99.588 | 100 | 1.3 | 1.25 | 98 | 101 |
| 180 | 98.346 | 100.248 | 96.985 | 101.368 | 99.612 | 99.523 | 99 | 1.5 | 1.53 | 97 | 101 |
| 195 | 98.972 | 100.403 | 96.721 | 101.510 | 99.838 | 99.270 | 99 | 1.6 | 1.62 | 97 | 102 |

TABLE 44
Dissolution results for Batch 7-4 (17% GR coating)
Batch 7-4 17% coating GR

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0.0 | 0 | | |
| 60 | 0.189 | 0.199 | 0.654 | 0.209 | 0.422 | 0.230 | 0 | 0.2 | 58.84 | 0 | 1 |
| 120 | 0.000 | 0.000 | 0.310 | 0.107 | 0.146 | 0.105 | 0 | 0.1 | 102.82 | 0 | 0 |
| 125 | 0.000 | 0.000 | 0.157 | 0.000 | 0.000 | 0.000 | 0 | 0.1 | 244.95 | 0 | 0 |
| 130 | 0.000 | 1.980 | 13.827 | 34.699 | 0.292 | 0.000 | 8 | 13.9 | 164.44 | 0 | 35 |
| 135 | 92.967 | 92.161 | 86.572 | 92.421 | 91.916 | 96.507 | 92 | 3.2 | 3.46 | 87 | 97 |
| 140 | 97.337 | 99.148 | 93.791 | 96.815 | 98.291 | 100.584 | 98 | 2.3 | 2.38 | 94 | 101 |
| 150 | 99.420 | 101.356 | 101.774 | 99.374 | 101.923 | 102.275 | 101 | 1.3 | 1.28 | 99 | 102 |
| 165 | 99.801 | 101.962 | 102.151 | 99.616 | 102.593 | 102.862 | 101 | 1.4 | 1.40 | 100 | 103 |
| 180 | 100.190 | 102.254 | 102.404 | 99.995 | 102.997 | 103.323 | 102 | 1.4 | 1.40 | 100 | 103 |
| 195 | 100.302 | 102.548 | 102.859 | 101.174 | 103.192 | 103.472 | 102 | 1.2 | 1.22 | 100 | 103 |

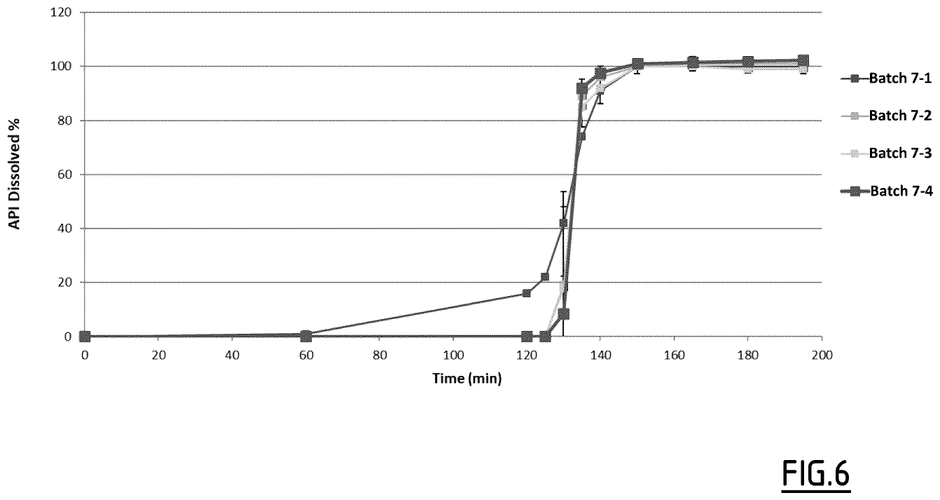

An overlay of dissolution profiles for Batches 7-1-7-4 is provided in FIG. 6.

### Content Uniformity—Batch 1-3

Content of uniformity studies were performed on 10 units with isocratic mode for Batch 1-3 (15% GR). The acceptance value (AV) was calculated, according the Eur. Ph. 2.9.40/USP<905> on n=10 units. The content uniformity complied with the specification of Ph.Eur. 2.9.40/USP <905> (≤15.0). Results obtained are provided in Table 45, and match the observation made on dissolution results. There was no variability between tablets and a target at 100%. The content uniformity comply with the specification of Ph.Eur. 2.9.40/USP <905> (≤15.0).

TABLE 45
Content uniformity results for batch 1-3 (15% GR).

| | | Batch 1-3 (20 mg |
|---|---|---|
| Content (%) | Specification | pitolisant HCl) |
| Tablet 1 | Eur. Ph. 2.9.40 | 98.8 |
| Tablet 2 | USP <905> | 101.9 |
| Tablet 3 | AV ≤15.0 with | 97.9 |
| Tablet 4 | L1 = 15.0 | 100.5 |
| Tablet 5 | L2 = 25.0 | 99.4 |
| Tablet 6 | | 98.9 |
| Tablet 7 | | 99.7 |
| Tablet 8 | | 100.1 |
| Tablet 9 | | 101.6 |
| Tablet 10 | | 99.2 |
| Mean value (%) | | 99.8 |
| SD | | 1.2 |
| Acceptance value (AV) | | 3.0 |

### Content Uniformity—Batches 3-6

The content of uniformity was performed on 10 units with gradient mode for enteric coated tablets of Batches 3, 5, and 6. The acceptance value (AV) was calculated, according the Eur. Ph. 2.9.40/USP<905> on n=10 units. The content uniformity results presented in Table 46 comply with the specification of Ph.Eur. 2.9.40/USP <905> (≤15.0).

TABLE 46
Content uniformity results for batches 3-6 (20 mg pitolisant HCl).

| Content (%) | Specification | Batch 3 | Batch 4 | Batch 5 | Batch 6 |
|---|---|---|---|---|---|
| Tablet 1 | Eur. Ph. 2.9.40 | 100.0 | 99.0 | 96.6 | 98.5 |
| Tablet 2 | USP <905> | 93.3 | 99.8 | 99.3 | 100.0 |
| Tablet 3 | AV ≤15.0 with | 98.3 | 96.9 | 99.5 | 96.6 |
| Tablet 4 | L1 = 15.0 | 97.4 | 100.6 | 96.2 | 99.9 |
| Tablet 5 | L2 = 25.0 | 98.8 | 97.6 | 95.7 | 99.6 |
| Tablet 6 | | 99.4 | 98.3 | 98.1 | 99.8 |
| Tablet 7 | | 100.0 | 101.9 | 98.4 | 101.4 |
| Tablet 8 | | 96.2 | 98.3 | 98.3 | 99.1 |
| Tablet 9 | | 99.0 | 98.0 | 99.5 | 96.2 |
| Tablet 10 | | 100.8 | 99.4 | 97.8 | 99.3 |
| Mean | | 98.3 | 99.0 | 97.9 | 99.0 |
| value (%) | | | | | |
| SD | | 2.2 | 1.5 | 1.4 | 1.6 |
| Acceptance | | 5.5 | 3.6 | 3.8 | 3.8 |
| value (AV) | | | | | |

### Appearance Studies

The appearance study was performed on n=10 tablets taken from a representative sample, by visual inspection, using Batches 3, 5, and 6. The results are provided in Table 47.

TABLE 47
Appearance for Batches 3, 5, and 6

| | Specification | Batch 3 | Batch 5 | Batch 6 |
|---|---|---|---|---|
| Appearance | White to almost white | complies | complies | complies |
| | tablet, no damage, | | | |
| | no sticking trace | | | |

### Average Mass Studies

Average mass studies were performed on n=10 tablets from a representative sample, by weighing, using Batches 3, 5, and 6. The results are provided in Table 48.

TABLE 48
Average mass results for batches 3, 5, and 6

| | Specification | Batch 3 | Batch 5 | Batch 6 |
|---|---|---|---|---|
| Average | Run and record | 147.3 | 142.0 | 151.6 |
| mass | for information | | | |

### Water Content Studies

The water content of a representative sample was performed (n=3) by volumetric Karl Fischer analysis. Batches 3, 5, and 6 were studied. The results are provided in Table 49.

TABLE 49
Water content for Batches 3, 5, and 6

| | Specification | Batch 3 | Batch 5 | Batch 6 |
|---|---|---|---|---|
| Mean water | Run and record | 3.8 | 3.8 | 3.4 |
| content (n = 3) | for information | | | |

### Assay and Purity

Assay and related substances testing was performed on n=2 samples, with Batches 3, 5, and 6, using HPLC according to the parameters provided in Table 50. A gradient method was developed in order to optimize a co-elution between API (pitolisant) and triethyl citrate (TEC), an excipient present in some formulations.

TABLE 50
HPLC parameters

| Parameters | Conditions | | |
|---|---|---|---|
| Instrument | HPLC system with UV detection, a heater column | | |
| | oven | | |
| Column | Luna II C18 Column, 250*4.6 mm, 5.0 μm particle | | |
| | size | | |
| Mobile phase | Mobile phase A: triethylamine 0.6% adjusted at pH | | |
| | 6.0 ± 0.05 | | |
| | Mobile phase B: acetonitrile | | |
| Wavelength | 220 nm | | |
| Flow rate | 1.4 mL/min | | |
| Column | 38° C. ± 2° C. | | |
| temperature | | | |
| Autosampler | 20° C. ± 3° C. | | |
| temperature | | | |
| Injection volume | 25 μL | | |
| Injection mode | Syringe draw rate mode normal | | |
| Needle cleaning | Acetonitrile/water (50 v/50 v) | | |
| solvent | | | |
| | | Mobile phase | Mobile phase |
| | Time (min) | A (%) | B (%) |
| Gradient | 0 | 65 | 35 |
| | 17 | 50 | 50 |
| | 30 | 45 | 55 |
| | 31 | 65 | 35 |
| | 36 | 65 | 35 |

| | |
|---|---|
| Retention time | Approx. min |
| of pitolisant | |
| Run time | 36 min |

Assay and related substances results complied with specifications. The target of 100% assay was reached and individual impurity as well as total impurities remained <LQ. The assay and purity results are provided in Table 51.

TABLE 51
Assay and purity results.

| | | | |
|---|---|---|---|
| Specification | Batch 3 | Batch 5 | Batch 6 |

Assay (%)

| | | | | |
|---|---|---|---|---|
| Sample 1 | 95-105 | 98 (98.3) | 97 (97.0) | 98 (98.0) |
| (% LC) | | | | |
| Sample 2 | | 98 (98.0) | 98 (98.1) | 97 (96.7) |
| (% LC) | | | | |
| Mean assay | | 98 (98.2) | 98 (97.5) | 97 (97.3) |
| (% LC) | | | | |

Related substances (%)

| | | | | |
|---|---|---|---|---|
| Each impurity | BF4 ≤0.2% | <LQ | <LQ | <LQ |
| | BP2.941 | ND | ND | ND |
| | (N-oxide ≤0.2% | | | |
| | Unknown | RRT 0.60: | RRT 0.60: | RRT 0.60: |
| | impurity ≤0.2% | 0.1 (0.05) | 0.1 (0.05) | 0.1 (0.05) |
| Total | Sum of | 0.1 | 0.1 | 0.1 |
| degradation | impurities | | | |
| products | (except BF6) | | | |
| | ≤1.0% | | | |

LQ: limit of quantification (0.05%).

### Summary of Quality Control Studies

For Batch 1 (amb II 3%, TEC 12%) all the results were compliant regardless of the amount of ACRYL-EZE® coating (10%, 12%, or 15%). Batch 4 (ACRYL-EZE® 10%, amb II 5%, PEG 8%) disintegration and dissolution results obtained were not compliant (disintegration: in acid media, 5 units were open after 120 mins; dissolution: all units exceed 10% dissolved after 120 min). On the other hand, Batches 3, 5, and 6 were compliant. In acid medium, the best formula obtained was Batch 3 (ACRYL-EZE® 15%, amb II 3%, PEG 8%). For Batches 3-6 the content uniformity tests were compliant, CU results showed good homogeneity of the batch. The results are summarized in Table 52 below.

TABLE 52
Initial analysis of Batches 3-6 summary.

| Initial analysis | | Batch 3 | Batch 4 | Batch 5 | Batch 6 |
|---|---|---|---|---|---|
| Experience | | Experience 2 | Experience 3 | Experience 5 | Experience 8 |
| ACRYL-EZE ® 93A19346 clear | | 15% | 10% | 10% | 15% |
| OPADRY ® amb II 88A190022 clear | | 3% | 5% | 3% | 5% |
| Plasticizer type | | PEG-8% | PEG-8% | TEC-12% | TEC-12% |
| Disintegration | Results in HCl 0.1M for 2 hours | Complies | Not | Complies | Complies |
| (n = 6) | without disks: | | compliant | | |
| | After 1 hour | NA | / | NA | NA |
| | After 2 hours | NA | 5 open | NA | 2 tablets |
| | | | | | swollen |
| | in Buffer Stage (pH 6.8) for 1 hour | 8 min | 7 min | 4 min | 8 min |
| | (with disks) | | | | |
| | [Maximum in Min] | | | | |
| Dissolution | Results (% dissolved) | Complies | Not | Complies | Complies |
| | | | compliant | | |
| | in acid (HCl 0.1M) after 120 | 0 (6) | 64 (5) | 3 (6) | 0 (6) |
| | minutes | [0-0] | [29-106]* | [0-5] | [0-1] |
| | Average (n) [min-max] | | | | |
| | in Buffer Stage (pH 6.8) | 99 (6) | 99 (5) | 99 (6) | 99 (6) |
| | Average (n) [min-max] | [96-101] | [98-102]* | [95-101] | [97-101] |
| CU | AV (n = 10) | 5.5 | 3.6 | 3.8 | 3.8 |
| | Mean (%) | 98.3 | 99.0 | 97.9 | 99.0 |
| Water content | Mean (%) | 3.8 | Not | 3.8 | 3.4 |
| | | | performed | | |

According to these initial analysis results, batches 3, 5, and 6 were put in stability studies. On the basis of Batch 3, additional trials were performed.

Batch 7 was also analyzed with the colored OPADRY® amb II for supportive batch (BEIGE) and to study the variation of ACRYL-EZE® coating quantity. Disintegration studies with Batch 7 showed a compliance of all these batches to the specification with an increase of the disintegration time the more ACRYL-EZE® coating there is in the formulation.

### Example 6. 1-Month Stability Test of Enteric-Coated Dose Form of Pitolisant HCl

Following a similar protocol as described in Examples 1 and 2, a cGMP batch of enteric coated 20 mg pitolisant HCl tablets was produced (Batch 8), as well as enteric coated 5 mg pitolisant HCl tablets (Batch 9).

The formula for Batch 8 and 9 are provided in Table 53.

TABLE 53
Exemplary enteric-coated dose forms with 4.45 mg and 17.8 mg pitolisant HCl

| Batch 8 (20 mg pitolisant HCl) | | | Batch 9 (5 mg pitolisant HCl) | |
|---|---|---|---|---|
| | Unit formula | | Unit Formula | |
| Raw Material | (17.8 mg) | | (4.45 mg) | |
| Internal phase | mg/unit | % w/w | mg/unit | % w/w |
| Pitolisant HCl | 20 | 16 | 5 | 16 |
| Microcrystalline cellulose | 81 | 64.8 | 20.25 | 64.8 |
| (Vivapur 102) | | | | |
| Crospovidone | 10 | 8 | 2.5 | 8 |
| (Polyplasdone XL) | | | | |
| Talc | 10 | 8 | 2.5 | 8 |
| Magnesium stearate | 3 | 2.4 | 0.75 | 2.4 |
| (Kemub EM-F-V) | | | | |
| Colloidal silica | 1 | 0.8 | 0.25 | 0.8 |
| Core-TOTAL | 125 | 100 | 31.25 | 100 |
| Coating | | | Coating | |
| Opadry AMB II¹ | 3.75 | | 0.9375 | |
| ACRYL-EZE ® clear² | 19.3125 | | 4.828125 | |
| PEG³ | 1.545 | | 0.38625 | |
| Sub-Total | 24.6075 | | 6.151875 | |
| Total | 149.61 | | 37.4025 | |

¹15% suspension, weight gain = 5%;
²20% suspension, weight gain = 15%;
³PEG at 8% of ACRYL-EZE ®.

A representative sample of Batch 8 tablets were subjected to 1 month stability testing. The results are provided in Table 54.

TABLE 54
1 month stability testing of enteric-coated Batch 8 tablets.

| Batch Number | Batch 8 | | Initial | 1 month | |
|---|---|---|---|---|---|
| Packaging: | Description | Clinical | 5° C. | 25° C./60% | 40° C./75% |
| 30 tablets/20 mL HDPE | | batch; | | RH | RH |
| bottle and PP caps with | | stability | | | |
| desiccant (2.4 g) | | | | | |
| TEST | ACCEPTANCE CRITERIA | RESULTS | | | |
| Appearance | Light purple round | Complies | Complies | Complies | Complies |
| | shape tablet engraved | | | | |
| | with “C” | | | | |
| Identification | Complies with reference | Complies | Complies | Complies | Complies |
| (pitolisant | | | | | |
| hydrochloride) | | | | | |
| Uniformity of dosage | L1 = 15.0 | Complies | NT | NT | NT |
| units | L2 = 25.0 | | | | |
| Water content by KF (%) | Report | 3.9% | 3.9% | 3.8% | 3.6% |
| In-vitro dissolution (%) | Acid stage: 2 h-no | Complies | Complies | Complies | Complies |
| | individual unit >10% | | | | |
| | Buffer stage: | 100% (n = 6) | 100 (6) | 99 (6) | 100 (6) |
| | Stage 1 no unit ≤ + 5% | [98-102] | [98-103] | [98-100] | [97-101] |
| | (80%) after 30 min | | | | |
| | Stage 2 and 3: see Eur. | | | | |
| | Ph. 2.9.3./USP <711> | | | | |
| Assay (pitolisant) (%) | 95-105% | 99% | 99% | 99% | 99% |
| Degradation products | Report ≤0.05% | | <LD | <LD | <LD |
| by LC-UV (%) | | | | | |
| BF4 | ≤0.2% | <LD | | | |
| BF2.941 | ≤0.3% | <LD | | | |
| Any unknown impurity | ≤0.2% | <LQ | | | |
| Total impurities | ≤1.0% | <LQ | | | |
| % Microbiological | | | | | |
| Quality | NMT 10³ CFU/g | <500 CFU/g | NT | NT | NT |
| TAMC | NMT 10² CFU/g | <10 CFU/g | | | |
| TYMC | Absence in 1 g | | | | |
| Escherichia coli | | Absence in | | | |
| | | 1 g | | | |

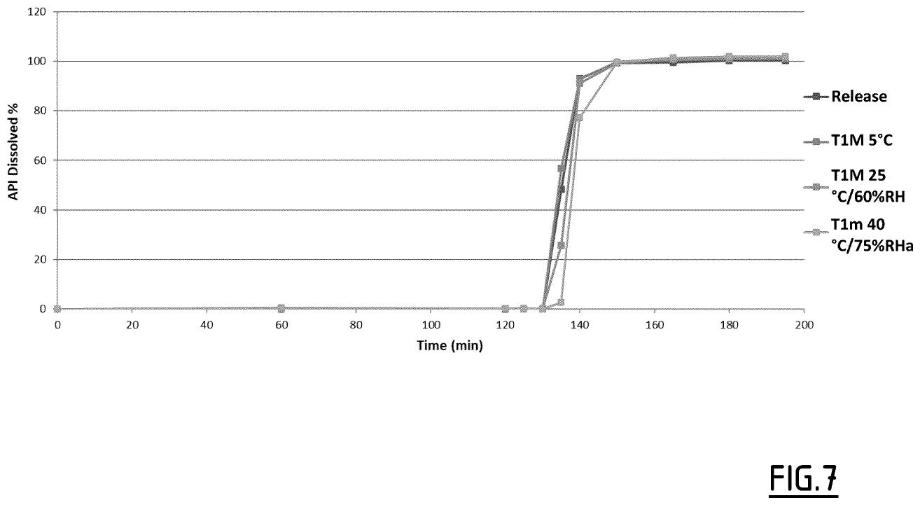

Additioal data is provided in FIG. 7, showing the stability dissolution profiles (means) of exemplary enteric-coated dose form (Batch 8) over 1 month, at 5° C., 25° C./60% RH, and at 40° C./75% RH.

### Example 7. Manufacture of Exemplary Enteric-Coated Dosage Forms

Gastroresistant (GR) film coated tablets for the supply of a phase I clinical study (NF2 project) were manufactured. Tablets were packaged into 20 mL HDPE bottle (30 tablets per bottle) closed with PP cap fitted with desiccant 2.4 g. A batch size of 5000 g core tablets was manufactured (4519.9 g were actually obtained).

At first, two sub-lots of coating were performed with Opadry AMBII Purple. Following slight difference in color between sub-lot 1 and 2, a third sub-lot was performed as an evaluation of color tone for information purposes but was not pursued. It was inconclusive why differences in color tone were obtained when the preparation and parameters were similar between the sub-lots. The differences in tone of color were cosmetic and did not impact the critical quality attributes of the gastric resistant tablet.)

The following raw materials were used: crospovidone (Polyplasdone XL) (supplier batch number: 0002618775); Anhydrous colloidale silica (Aerosil 200 pharma) (supplier batch number: M22120016), Opadry amb II Purple, 88A200015 (supplier batch number: DT743323); Acryl eze 93A19346 clear (supplier batch number: DT738120), and cellulose microcrystalline (Vivapur 102) (supplier batch number: 56102212111). Following manufacturing, all of these raw materials were released and found compliant to the required quality standard. There was no impact on clinical batch.

### Manufacturing Formula

The actual manufacturing formula of core tablets (active batch, LC23120) is described below in Table 55. For readjustment, pitolisant HCl assay and water content (98.19%) were taken into account (compensation on cellulose). The equipment used is described above in Materials and Methods.

TABLE 55
Manufacturing formula for LC23120 clinical batch.

| | | Theoretical | Actual |
|---|---|---|---|
| | Manufacturer | quantity | quantity |
| Raw material | batch No. | (g) | (g) |
| Pitolisant HCl | 001231789 | 814.75 | 814.07 |
| Colloidal silica (aerosil 200 pharma) | M22120016 | 40.00 | 39.93 |
| Crospovidone (polyplasdone XL) | 0002618775 | 400.00 | 400.4 |
| Microcrystalline cellulose | 56102212111 | 3225.25 | 3225.2 |
| (vivapur 102) | | | |
| Talc (Luzenac 00) | M21040260 | 400.00 | 400.3 |
| Magnesium stearate | 139721 | 120.00 | 120.48 |
| (Kemilub EM-F-V) | | | |
| TOTAL | / | 5000.00 | 5000.38 |

The manufacturing parameters used during blending step are provided in Table 56. The blend was sampled 5 times for blend uniformity (2 top, 1 middle, 2 bottom; about 625 mg each). Filling level was 30%, which is suitable for good mixing.

TABLE 56
Manufacturing parameters for active batch LC23120.

| | | Required | Actual |
|---|---|---|---|
| Blending step | Parameters | value | value |
| Raw materials sieving | Sieve aperture | 800 μm | 800 μm |
| (about 50% cellulose + | | | |
| crospovidone + talc + | | | |
| API + about 50% cellulose) | | | |
| Blend 1 (about 50% | Blending time | 3.63* | 3.63 |
| cellulose + crospovidone | | | |
| +talc + API + about | Blending speed | 22 | 22 |
| 50% cellulose) | | | |
| Raw materials sieving | Sieve aperture | 800 μm | 800 μm |
| (silica and magnesium | | | |
| stearate) | | | |
| Blend 2 (silica + | Blending time | 3.63* | 3.63 |
| magnesium stearate | Blending speed | 22 | 22 |
| | Filling level | For | 30% |
| | | information | |

*3.63 (min, cent) corresponding to 03 min 38 s at 22 rpm, i.e. 80 rotations.

The yield of blending step is presented in Table 57 and is very good (98.5%).

TABLE 57
Blending step yield.

| | Verum batch |
|---|---|
| Attributes | LC23120 |
| Actual used quantity (g) | 5000.38 |
| Obtained quantity (g) | 4926.7 |
| Waste quantity (g) | 0 |
| Step yield (%) | 98.5 |

Flowability and density of the final blend (25 g) was tested. While flowability was characterized as poor, there was no issue during tableting. Relevant parameters are provided in Table 58.

TABLE 58
Flowability and density data.

| | Final blend |
|---|---|
| | LC23120 |
| Sample size [g] | 24.9 |
| Flowability | |
| Flowing time [s] | 02 |
| Flowing speed [g/s] | 12.5 |
| Tapped volume [mL] | |
| V0 | 64 |
| V10 | 60 |
| V50 | 48 |
| 0 | 46 |
| V12 | / |
| 50 | / |
| V25 | |
| 00 | |
| V10- V500 | |
| Density [g/mL] | |
| D0 | 0.39 |
| D10 | 0.42 |
| D50 | 0.52 |
| 0 | 0.54 |
| D12 | / |
| 50 | |
| D2500 | |
| Carr index | 28 |
| Hausner index | 1.39 |
| Flow character | Poor |
| LOD [%] | 3.74 |

### Tableting Step

The tableting was performed on a rotative tableting press (Fette P1200), using 8 punches.

Table 59 shows tableting parameters and obtained IPC results on tablet mass, hardness and thickness. Results of friability and disintegration tests are showed in Table 60.

TABLE 59
Tableting parameters and IPC results.

| | |
|---|---|
| | Core tablets |
| Specifications | LC23120 |

Tableting actual parameters

| | | |
|---|---|---|
| Tableting speed (tab/h) | 90 000 to confirm | 90 000 |
| Filling height (mm) | / | 7.17 |
| Feeder speed (rpm) | 20 rpm to confirm | 40* |
| Mean compression force (kN) | About 5 ± 1 KN (to confirm) | 5.8 kN |
| Srel (%) | | 5.0% |
| Mean pre-comp. force (kN) | About 1.5 ± 0.5 KN (to confirm) | 1.5 |
| Tableting run duration (min) | / | 72 |

IPC results

| | | |
|---|---|---|
| Mean tablet mass (mg) | 125.0 ± 4.0% (120.0-130.0) | 124.9 |
| Min-Max mean tablet mass (mg) | T1: 125.0 ± 7.5% (115.6-134.4) | 123.3-125.8 |
| Min-Max individual tablet mass (mg) | T2: 125.0 ± 15.0% (106.3-143.8) | 121.2-128.2 |
| CV (%) | | 0.80 |
| Mean tablet hardness (N) | About 55N (for information) | 56 |
| Min-Max mean hardness | | 53-60 |
| Min-Max indiv. Hardness | | 49-66 |
| CV (%) | | 4.20 |
| Mean tablet thickness (mm) | For information | 3.20 |
| Min-Max mean thickness | | 3.15-3.23 |
| Min-Max indiv. Thickness | | 3.13-3.25 |
| CV (%) | | 1.08 |

*At 15 minutes, the feeder speed was increased from 20 to 40 rpm.

TABLE 60
Disintegration and friability tests results.

| | | Core tablets LC23120 | | |
|---|---|---|---|---|
| | | Start | Middle | End |
| Disintegration time [min/s] | Min | 04 min 59 s | 02 min 57 s | 04 min 45 s |
| Specification: ≤15 min (target around 04 min) | Max | 05 min 35 s | 05 min 21 s | 05 min 07 s |
| Friability [%] | 100 rots. | 0.0% | 0.0% | 0.0% |
| Specification: ≤0.3% (target: ≤0.1%) | | | | |
| LOD [%] for information | / | 4.08 | 3.68 | 3.72 |

The results of disintegration and friability IPC tests comply with the specifications. LOD is quite high, which is known with this product.

Tableting yield was more than 90%, which is very good (see Table 61A). Core tablets were split in three sub-lots for coating: 1004.8 g for sub-lot 1, 1004.8 g for sub-lot 2 and 1000.6 g for sub-lot 3 (which was ultimately not pursued further).

TABLE 61A
Tableting and global assessment.

| | Core tablet |
|---|---|
| Item | LC23120 |
| Implemented quantity (g) | 4926.7 |
| Obtained quantity (g) | 4519.9 |
| Core tablet mean mass (mg) | 124.8 |
| Number of units obtained | 36 217 |
| Waste quantity (g) | 352.8 |
| Tableting yield (%) | 91.7 |

### Coating Step

The coating was performed in 330 mm perforated pan using spray nozzle of 0.8 mm. Two sub-lots were film coated in two steps: a first layer of Opadry amb II was performed (sub layer) with weight gain at 3%. Then, the gastro resistant coating was performed with acryleze with weight gain at 15%. The suspensions were prepared in excess (×2.5 for ambII, ×2.0 for acryl eze) at 15% or 20% solids for ambII and acryl eze, respectively (PEG corresponding at 8% of acryl eze). Two sub-lots of 1000 g (1004.8 g for sub-lots 1 and 2) were performed. Details are in Tables 61B and Table 62.

TABLE 61B
Suspension preparation (amb II)

| | | | Actual | Actual |
|---|---|---|---|---|
| | | Theoretical | quantity | quantity |
| | | quantity | (g) | (g) |
| Raw material | Batch No. | (g) | Sub-lot 1 | Sub-lot 2 |
| Opadry amb II purple | ME-23-033 | 75.00 | 75.10 | 75.00 |
| 88A200015 | | | | |
| Water for injection | ME-22-064 | 425.00 | 425.3 | 426.6 |
| TOTAL | LC23120 | / | / | / |

TABLE 62
Suspension preparation (amb II)

| | | LC23120 | LC23120 |
|---|---|---|---|
| Step | Parameters | sub-lot 1 | Sub-lot 2 |
| Amb II | Stirring speed | 750-850 rpm | 750-850 rpm |
| introduction | stirring time | 02 min 50 sec | 02 min 30 sec |
| Stirring | Stirring speed | 750-850 rpm | 750-850 rpm |
| | stirring time | 50 min | 50 min |

Amb II coating lasted 90 minutes for sub-lot 1 and 80 minutes for sub-lot 2. During sub-lot 1, spray gun was clogged after 20 minutes. Spraying was stopped and pan speed was decreased at 15 rpm during gun cleaning. Coating was re-started after 3 minutes. After 50 minutes of coating, pan speed was decreased from 28 to 26 rpm to avoid tablet loss on the side.

No issue was observed during sub-lot 2.

Coating parameters were close to target for both sub-lots and similar between sub-lots 1 and 2—details in Tables 63A and 63B.

TABLE 63A
Coating parameters (amb II).

| | | LC23120 | LC23120 |
|---|---|---|---|
| | | sub-lot 1 | sub-lot 2 |
| Pre heat (min) | | 13 | 9 |
| Product | Start | 20.9 | 21.2 |
| temperature (° C.) | End | 45.6 | 45.0 |
| Pan speed | Start | 4.0 [intermittent] | 4.0 [intermittent] |
| (rpm) | End | 4.0 [intermittent] | 4.0 [intermittent] |
| Actual inlet air | Start | 67.2 | 67.1 |
| temperature (° C.) | End | 65.9 | 66.3 |
| Outlet air | Start | 48.5 | 50.5 |
| temperature (° C.) | End | 53.8 | 51.6 |
| Air flow rate | Start | 316 | 296 |
| (m³/h) | End | 298 | 308 |

TABLE 63B
Coating parameters (amb II) continued.

| | | | LC23120 | LC23120 |
|---|---|---|---|---|
| | | Specifications | sub-lot 1 | sub-lot 2 |
| Coating time (min) | | | 90 | 80 |
| Product | Min | 45-48 | 45.6 | 45.0 |
| temperature (° C.) | Max | | 47.3 | 46.0 |
| Pan speed (rpm) | Min | 25 ± 5 | 26.0 | 26.0 |
| | Max | | 28.0 | 26.0 |
| Actual inlet air | Min | 66 ± 2 | 65.6 | 66.6 |
| temperature (° C.) | Max | | 67.7 | 67.2 |
| Outlet air | Min | | 52.0 | 52.0 |
| temperature (° C.) | Max | | 54.3 | 52.9 |
| Air flow rate | Min | 300 ± 5 | 293 | 292 |
| (m³/h) | Max | | 308 | 304 |
| Atomization pressure (bar) | | 1.2 ± 0.5 | 1.2 | 1.2 |
| Spray width (bar) | | 1.6 ± 0.5 | 1.6 | 1.6 |
| Spraying flow rate | Min | 3.0 ± 1.0 | 3.26 | 3.00 |
| (g/min) | Max | | 4.07 | 3.95 |
| Pump setting (%) | | 10 | 9-10 | 10 |
| Drying time (min) | | | 5 | 5 |
| Product | Start | | 47.3 | 46.0 |
| temperature (° C.) | End | | 50 | 49.4 |
| Pan speed (rpm) | Start | 15 | 15.0 | 15.0 |
| | End | | 15.0 | 15.0 |
| Actual inlet air | Start | | 66.1 | 67.6 |
| temperature (° C.) | End | | 66.0 | 66.6 |
| Outlet air | Start | | 52.8 | 53.4 |
| temperature (° C.) | End | | 54.6 | 53.8 |
| Air flow rate | Start | | 300 | 299 |
| (m³/h) | End | | 297 | 292 |
| Weight gain (%) | | | 3.4 | 3.0 |
| Cooling time (min) | | | 17 | 20 |
| Product | Start | <35 | 50.0 | 49.5 |
| temperature (° C.) | End | | 34.9 | 34.9 |
| Pan speed (rpm) | Start | 4 | 4.0 [intermittent] | 4.0 [intermittent] |
| | End | | 4.0 [intermittent] | 4.0 [intermittent] |
| Actual inlet air | Start | | 65.9 | 66.3 |
| temperature (° C.) | End | | 41.8 | 37.9 |
| Outlet air | Start | | 52.2 | 54.0 |
| temperature (° C.) | End | | 36.4 | 36.3 |
| Outlet air temperature | Start | | 299 | 302 |
| (° C.) | End | | 309 | 308 |

For acryl eze coating, suspensions were prepared according to Tables 64 and 65.

TABLE 64
Suspension preparation (acryl eze).

| | | | Actual | Actual |
|---|---|---|---|---|
| | | Theoretical | quantity (g) | quantity (g) |
| Raw material | Batch No. | quantity (g) | Sub-lot 1 | Sub-lot 2 |
| Acryl eze clear | ME-22-083 | 300.00 | 300.4 | 300.2 |
| 93A19346 | | | | |
| PEG 8000 | ME-22-073 | 24.00 | 23.98 | 24.11 |
| Water for | | 1200.00 | 1200.2 | 1201.7 |
| injection | | | | |
| TOTAL | LC23120 | | | |

TABLE 65
Suspension preparation (acryl eze)

| | | LC23120 | LC23120 |
|---|---|---|---|
| Step | Parameters | sub-lot 1 | Sub-lot 2 |
| Amb II | Stirring speed | 1300 rpm | 1200-1300 rpm |
| introduction | stirring time | 1 min + 5 min | 54 sec + 5 min |
| Stirring | Stirring speed | 1300 rpm | 1300-1900 rpm |
| | stirring time | 3 + 60 min | 02 min 48 s + 73 min |

The coating was performed in 330 mm perforated pan using spray nozzle of 0.8 mm. Sub-lot 1 lasted 207 minutes whereas sub-lot 2 lasted 175 minutes. During sub-lot 1, there was an issue with peristaltic pump after 180 minutes of coating. The spraying was stopped to clean the gun. Coating was re-start after few minutes. There was no particular issue during sub-lot 2. Coating parameters were close to target for the two sub-lots and similar between sub-lots 1 and 2, with details in Tables 66A and 66B.

TABLE 66
Coating parameters (acryl eze)

| | | Specifications | LC23120 sub-lot 1 | LC23120 sub-lot 2 |
|---|---|---|---|---|
| Pre heat (min) | | | 5 | 6 |
| Product temperature (° C.) | Start | | 25.5 | 23.4 |
| | End | | 35.0 | 35.6 |
| Pan speed (rpm) | Start | 4 | 4.0 [intermittent] | 4.0 [intermittent] |
| | End | | 4.0 [intermittent] | 4.0 [intermittent] |
| Actual inlet air temperature | Start | | 44.1 | 44.0 |
| (° C.) | End | | 43.9 | 44.1 |
| Outlet air temperature (° C.) | Start | | 36.2 | 36.2 |
| | End | | 37.6 | 38.1 |
| Air flow rate (m³/h) | Start | | 306 | 303 |
| | End | | 300 | 296 |
| Coating time (min) | | | 207 | 175 |
| Product temperature (° C.) | Min | <35° C. | 32.8 | 32.5 |
| | Max | (target: 33° C.) | 34.5 | 33.9 |
| Pan speed (rpm) | Min | 25 ± 5 | 26.0 | 26.0 |
| | Max | | 26.0 | 26.0 |
| Actual inlet air | Min | 43 ± 2 | 43.5 | 43.8 |
| temperature (° C.) | Max | | 44.3 | 45.6 |
| Outlet air temperature (° C.) | Min | | 36.7 | 36.6 |
| | Max | | 37.5 | 38.3 |
| Air flow rate (m³/h) | Min | 300 ± 5 | 292 | 291 |
| | Max | | 308 | 311 |
| Atomization pressure (bar) | | 1.2 ± 0.5 | 1.2 | 1.2 |
| Spray width (bar) | | 1.6 ± 0.5 | 1.6 | 1.6 |
| Spraying flow rate (g/min) | Min | 3.0-5.5 ± 0.5 | 3.11 | 3.02 |
| | Max | | 5.31 | 6.00 |
| Pump setting (%) | | 10-15 | 10-14 | 10-14 |
| Drying time (min) | | | 5 | 5 |
| Product temperature (° C.) | Start | | 32.9 | 33.9 |
| | End | | 36.0 | 37.2 |
| Pan speed (rpm) | Start | 15 | 15.0 | 15.0 |
| | End | | 15.0 | 15.0 |
| Actual inlet air | Start | 43 ± 2 | 43.8 | 45.3 |
| temperature (° C.) | End | | 44.1 | 45.3 |
| Outlet air temperature (° C.) | Start | | 36.8 | 38.2 |
| | End | | 38.9 | 39.3 |
| Air flow rate (m³/h) | Start | 300 ± 5 | 307 | 303 |
| | End | | 292 | 306 |
| Weight gain (%) | | | 16.4 | 16.2 |
| Cooling time (min) | | | 8 | 19 |
| Product temperature (° C.) | Start | | 36.0 | 37.2 |
| | End | | 32.0 | 31.6 |
| Pan speed (rpm) | Start | 4 | 4.0 [intermittent] | 4.0 [intermittent] |
| | End | | 4.0 [intermittent] | 4.0 [intermittent] |
| Actual inlet air | Start | | 44.1 | 45.2 |
| temperature (° C.) | End | | 36.0 | 33.7 |
| Outlet air temperature (° C.) | Start | | 38.9 | 39.4 |
| | End | | 33.1 | 32.1 |
| Air flow rate (m³/h) | Start | 300 ± 5 | 292 | 303 |
| | End | | 297 | 298 |

The two sub-lots were compliant and had similar IPC results, as shown in Table 67.

TABLE 67
IPC results on film-coated tablets.

| | | | |
|---|---|---|---|
| | | LC23120 | LC23120 |
| IPC results | Specifications | Sub-lot 1 | Sub-lot 2 |

After ambII coating

| | | | |
|---|---|---|---|
| After ambII coating | | | |
| Mean tablet mass (mg) | 128.75 ± 5.0% (122.31-135.19) | 130.3 | 129.6 |
| Min-Max individual tablet mass (mg) | T1: 128.75 ± 7.5% (119.09-138.41) | 126.6-132.7 | 125.8-132.4 |
| CV (%) | T2: 128.75 ± 15.0% (109.44-148.06) | 1.26 | 1.40 |
| Mean tablet hardness (N) | For information | 69 | 67 |
| Min-Max indiv. Hardness | | 64-75 | 60-73 |
| CV (%) | | 4.61 | 5.56 |
| Mean tablet thickness (mm) | For information | 3.33 | 3.32 |
| Min-Max indiv. Thickness | | 3.27-3.37 | 3.25-3.35 |
| CV (%) | | 0.86 | 0.81 |
| LOD (%) | For information | 4.20 | 3.93 |

After acryl eze coating

| | | | |
|---|---|---|---|
| Mean tablet mass (mg) | 149.61 ± 5.0% (142.13-157.09) | 149.6 | 148.7 |
| Min-Max individual tablet mass (mg) | T1: 149.61 ± 7.5% (138.39-160.83) | 146.1-152.8 | 146.2-151.6 |
| CV (%) | T2: 149.61 ± 15.0% (127.17-172.05) | 1.18 | 0.90 |
| Mean tablet hardness (N) | For information | 128 | 119 |
| Min-Max indiv. Hardness CV (%) | | 114-142 | 114-125 |
| | | 5.40 | 2.60 |
| Mean tablet thickness (mm) | For information | 3.60 | 3.57 |
| Min-Max indiv. Thickness CV (%) | | 3.51-3.63 | 3.50-3.61 |
| | | 0.77 | 0.88 |
| LOD (%) | For information | 3.46 | 3.72 |
| Disintegration | No open tablet in HCl after 2 H | Compliant | Compliant |

Global yield was acceptable (Table 68).

TABLE 68
Coating assessment. Reconciliation was out of specifications due to an anomaly.

| Item | Code | LC23120 |
|---|---|---|
| Remaining suspension (ambII) sub-lot 1 (g) | (7) | 155.6 |
| Remaining suspension (ambII) sub-lot 2 (g) | (8) | 210.6 |
| Remaining suspension (acryl eze) sub-lot 1 (g) | (9) | 562.0 |
| Remaining suspension (acryl eze) sub-lot 2 (g) | (10) | 647.6 |
| Remaining suspension in solids (g) | 0.15*[(7) + (8)] + 0.20*[(9) + (10)] | 296.85 |
| Other waste (g) | | 550.4 |
| Total quantity of waste (g) | (1) | 847.25 |
| Sampling (g) | (2) | 112.9411 |
| Implemented quantity for blend (g) | (3) | 5000.38 |
| Implemented quantity for coating (g) | (4) | 798.79 |
| Implemented quantity for coating with core tablets (g) | (11) | 2808.39 |
| Obtained quantity sub-lot 1 (g) | | 1095.8 |
| Obtained quantity sub-lot 2 (g) | | 1038.4 |
| Total obtained quantity (g) | (5) | 2134.2 |
| Remaining of core tablets (g) | (6) | 2510.9 |
| Mean mass film coated tablet (mg) | | 149.15 |
| Obtained quantity (tab) | | 14309 |
| Coating yield (%) | (5)/(4)*100 | 76.0 |
| Global yield (%) | [(5) + (6)]/5393.76*100 | 86.1 |
| Reconciliation (%) | [(5) + (6) + (1) + (2)]/[(11) + (6)]*100 | 105.4 |

Batch LC23120 was packaged into 20 mL HDPE bottles (30 tablets per bottle) closed by PP childproof resistant caps with desiccant (2.4 g) (LC23120A1) and then labelled (LC23120A2). One cardboard box of 38 bottles was prepared.

Wakix comparator (manufacturer batch number 3831701) was also packaged into 20 mL HDPE bottles (30 tablets per bottle) closed by PP childproof resistant caps with desiccant (2.4 g) and labelled (LC23177A1). One cardboard box of 33 bottles was prepared.

Pantoprazole comparator (2 labelled blisters of 14 tablets per marketed box; manufacturer batch number 535260) was labelled (LC23206A1). Five boxes of 2 blisters each were prepared.

### Lubricated Blend Results

An analytical method for blend uniformity was caried out for sample preparations and quantification. As about 625 mg of powder were sampled, the preparation was identical to preparation of sample solution for 20 mg tablets.

As observed in Table 69, individual assay results on blends are homogeneous and near the target.

TABLE 69
Blend uniformity results on five units from top (n = 2), middle
(n = 1), and bottom (n = 2) of mix.

| | Specification | LC23120 |
|---|---|---|
| Blend 1 | 90.0-110.0% (report as individual results) | 99.5 |
| Blend 2 | RSD: not more than 5% | 99.3 |
| Blend 3 | | 99.8 |
| Blend 4 | | 99.6 |
| Blend 5 | | 100.4 |
| Average | | 99.7 |
| RSD | | 0.4 |

### Content Uniformity (Core Tablets)

The content of uniformity was performed on 10 units from start, 10 units from middle and 10 units from end of compression. The acceptance value (AV) was calculated, according the Ph. Eur. 2.9.40/USP <905>, on n=10 units.

The content uniformity results presented in Table 70 comply with the specification of Ph. Eur. 2.9.40/USP <905>, with an acceptance value≤15.0

TABLE 70
Content uniformity results on 10 units from
start, middle, and end of compression.

| | | LC23120 | | |
|---|---|---|---|---|
| | Specification | Start | Middle | End |
| Tablet 1 (%) | Eur. Ph. 2.9.40 | 98.217 | 101.289 | 101.827 |
| Tablet 2 (%) | USP <905> | 98.371 | 103.788 | 100.648 |
| Tablet 3 (%) | | 96.411 | 102.043 | 103.335 |
| Tablet 4 (%) | | 98.478 | 101.421 | 101.270 |
| Tablet 5 (%) | | 96.422 | 102.273 | 101.809 |
| Tablet 6 (%) | | 97.444 | 101.662 | 103.551 |
| Tablet 7 (%) | | 97.396 | 103.761 | 102.234 |
| Tablet 8 (%) | | 97.611 | 101.605 | 102.685 |
| Tablet 9 (%) | | 97.277 | 101.902 | 100.584 |
| Tablet 10 (%) | | 97.010 | 101.601 | 102.233 |
| Mean value (%) | | 97.5 | 102.1 | 102.0 |
| SD | | 0.7 | 0.9 | 1.0 |
| Acceptance value | | 2.8 | 2.8 | 2.9 |

### Bulk Analysis—Appearance

The appearance was performed (on n=10 tablets) from a representative sample, by visual inspection, on each of the two sub-lot. A difference of coloration between both sub-lots was observed. Nevertheless, results conform to purple to light purple appearance criteria.

TABLE 71
Appearance results for batch LC23120.

| | Specifica- | | |
|---|---|---|---|
| | tion | LC23120 sub-lot 1 | LC23120 sub-lot 2 |
| Appear- | For | Purple to light purple | Purple to light purple |
| ance | information | round shaped tablet | round shaped tablet |
| | | engraved with “C” | engraved with “C” |

### Bulk Analysis—Average Mass

For both sub-lot of batch LC23120, the average mass was performed (on n=10 tablets) from a representative sample, by weighing. Sub-lot 1 had a mean mass of 150.2 mg, and sub-lot 2 had a mean mass of 148.2 mg.

### Bulk Analysis—Water Content

For both sub-lots of batch LC23120, the water content was performed by Karl Fischer (on n=2 tablets) from a representative sample. The water content for sub-lot 1 was 4.2%, and the water content for sub-lot 2 was 4.0%.

### Bulk Analysis—Disintegration

Disintegration analysis was performed according to the Eur. Ph. 2.9.1 for sub-lot 1 and 2 of batch LC23120 during IPC (on n=6 tablets) and results were compliant.

The clinical batch LC23120, bulk of gastro resistant film coated tablet containing 20 mg of pitolisant hydrochloride, is composed of two sub-lots (as 2 runs of coating were performed, venue 1 and venue 2). During the manufacturing, it was observed that theses 2 venues had two slightly different colors (purple for venue 1 and light purple for venue 2). For investigations and to evaluate the impact, it was decided to perform disintegration time in HCL testing and QC dissolution testing for these two venues. Nevertheless, disintegration testing for sub-lot 1 and 2 of batch LC23120 were performed again during the initial analysis of the bulk tablets. The disintegration time was performed (on n=6 tablets) from a representative sample, for sub-lot 1 and 2 of batch LC23120, according to the Eur. Ph. 2.9.1. The disintegration results comply with the specifications for sub-lot 1, with no tablets opened in HCl 0.1M and are for information in phosphate buffer pH 6.8. However, disintegration results do not comply with the specifications for sub-lot 2 with 4 tablets opened in HCl 0.1M (see 011/RHS/2023).

During the 011/RHS/2023, the investigations show that the method cause (the GR tablets open in the presence of small amount of water) seems the most probable cause, but cannot be confirmed. The results of the initial analysis are not invalidated. A retest was performed on n=6 tablets for sub-lot 2 of batch LC23120 using careful cleaning and drying of the apparatus grid and the result was compliant. Before the test, the grid was washed, dried (with compressed air) and dipped in HCl during 3 seconds and 3 times (to remove residual water) before dropping the tablet on the grid. For disintegration time testing in HCl 0.1N for 2 hours, none of the tablets had shown sign of either disintegration or cracks that would allow the escape of the contents. Film coating barrier was not altered.

TABLE 72
Disintegration time results for batch LC23120 during IPC, initial analysis and retest.

| | | LC23120 sub-lot 1 | LC23120 sub-lot 2 |
|---|---|---|---|
| | Specification | (IPC) | (IPC) |
| Disintegration | First in HCl 0.1M for 2 hours (without | Complies | Complies |
| time | disks): | | |
| | No tablet opened | | |
| | Then phosphate buffer pH 6.8 for 60 | NA* | Max: 10 min |
| | minutes (with disks):* | | Min: 09 min |
| | For Information | | Mean: 10 min |

| | | LC23120 sub-lot 1 | LC23120 sub-lot 2 |
|---|---|---|---|
| | Specification | (initial analysis) | (initial analysis) |
| Disintegration | First in HCl 0.1M for 2 hours (without disks): | Complies | Not Complies |
| time | No tablet opened | | 4 tablets opened* |
| | Then phosphate buffer pH 6.8 for 60 minutes | Max: 11 min | Max: 10 min |
| | (with disks): | Min: 08 min | Min: 01 min |
| | For information | Mean: 10 min | Mean: 04 min |

| | | LC23120 sub-lot 2 |
|---|---|---|
| | Specification | (retest 011/RHS/2023) |
| Disintegration | First in HCl 0.1M for 2 hours (without disks): | Complies |
| time | No tablet opened | |
| | Then phosphate buffer pH 6.8 for 60 minutes | Max: 22 min |
| | (with disks): | Min: 09 min |
| | For Information | Mean: 13 min |

### Bulk Analysis—Dissolution

The dissolution testing was performed on sub-lot 1 and 2 of bulk film-coated tablets batch LC23120 from a representative sample. Results in Tables 73 and 74.

TABLE 73
Dissolution results for batch LC23120 sub-lot 1.
LC23120 Sub-lot 1 - F/23/053

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0.000 | 0.000 | 0.000 | 0.000 | 0.000 | 0.000 | 0 | 0.0 | 0.0 | 0 | 0 |
| 120 | 0.000 | 0.000 | 0.000 | 0.000 | 0.000 | 0.000 | 0 | 0.0 | 0.0 | 0 | 0 |
| 125 | 0.000 | 0.000 | 0.000 | 0.000 | 0.000 | 0.000 | 0 | 0.0 | 0.0 | 0 | 0 |
| 130 | 3.489 | 0.000 | 0.000 | 0.217 | 0.000 | 0.000 | 1 | 1.4 | 228.2 | 0 | 3 |
| 135 | 83.742 | 0.000 | 53.417 | 91.988 | 84.265 | 84.392 | 66 | 35.1 | 53.0 | 0 | 92 |
| 140 | 93.071 | 91.403 | 83.425 | 96.211 | 92.150 | 93.577 | 92 | 4.3 | 4.7 | 83 | 96 |
| 150 | 102.976 | 98.708 | 99.870 | 100.151 | 97.262 | 102.603 | 100 | 2.2 | 2.2 | 97 | 103 |
| 165 | 103.684 | 99.756 | 101.591 | 100.253 | 98.627 | 103.507 | 101 | 2.1 | 2.0 | 99 | 104 |
| 180 | 103.773 | 99.782 | 101.055 | 100.124 | 98.607 | 103.257 | 101 | 2.0 | 2.0 | 99 | 104 |
| 195 | 103.635 | 99.326 | 101.280 | 100.043 | 98.618 | 103.271 | 101 | 2.1 | 2.1 | 99 | 104 |

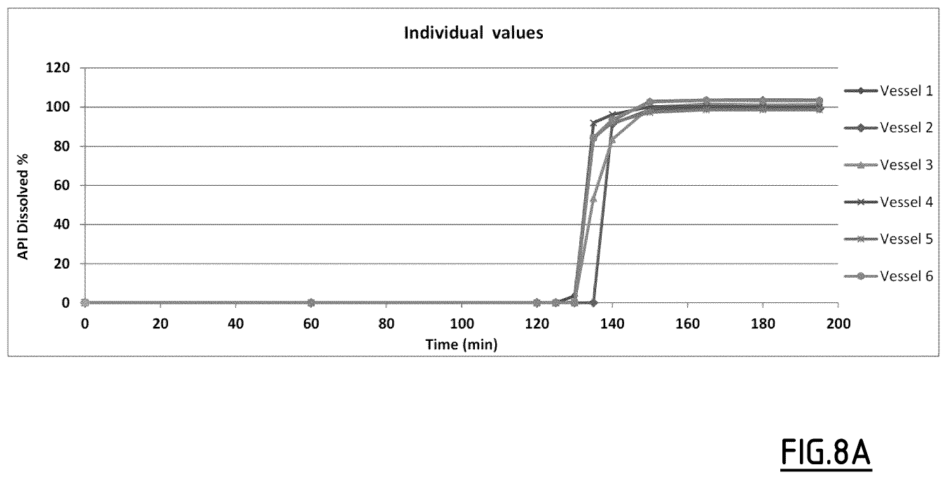

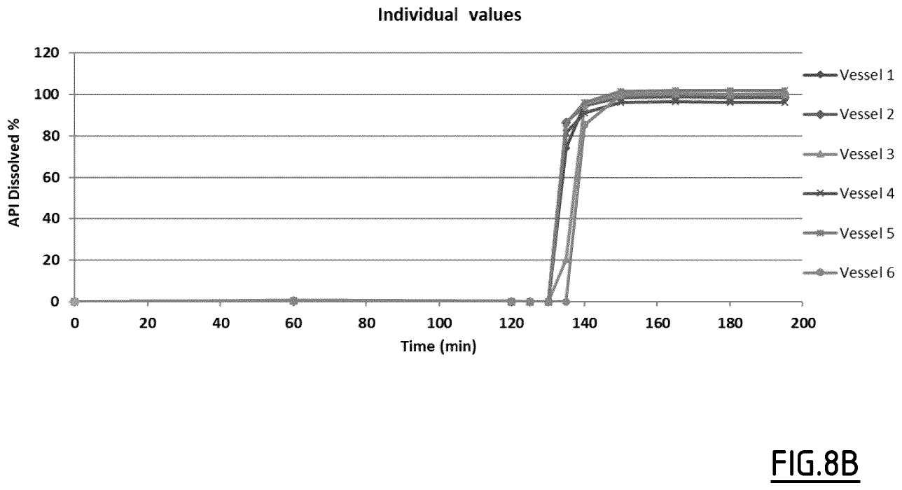

The dissolution profile for batch LC23120 sub-lot 1 is shown in FIG. 8A.

TABLE 74
Dissolution results for batch LC23120 sub-lot 2.
LC23120 sub-lot 2 - F/23/054

| Time | Vessel | Vessel | Vessel | Vessel | Vessel | Vessel | Mean | Std. | | | |
|---|---|---|---|---|---|---|---|---|---|---|---|
| (min) | 1 | 2 | 3 | 4 | 5 | 6 | (n = 6) | Dev. | RSD % | min | max |
| 0 | 0 | 0 | 0 | 0 | 0 | 0 | 0 | 0 | 0 | 0 | 0 |
| 60 | 0.310 | 0.274 | 0.281 | 0.314 | 0.717 | 0.311 | 0 | 0.2 | 46.7 | 0 | 1 |
| 120 | 0.099 | 0.104 | 0.111 | 0.114 | 0.194 | 0.108 | 0 | 0.0 | 29.4 | 0 | 0 |
| 125 | 0.000 | 0.000 | 0.000 | 0.000 | 0.000 | 0.000 | 0 | 0.0 | 0.0 | 0 | 0 |
| 130 | 0.000 | 0.000 | 0.000 | 0.000 | 0.000 | 0.000 | 0 | 0.0 | 0.0 | 0 | 0 |
| 135 | 74.081 | 86.526 | 20.591 | 81.448 | 85.947 | 0.000 | 58 | 37.9 | 65.2 | 0 | 87 |
| 140 | 95.345 | 94.616 | 95.313 | 91.135 | 96.218 | 85.385 | 93 | 4.1 | 4.4 | 85 | 96 |
| 150 | 99.200 | 98.625 | 99.787 | 96.136 | 101.539 | 99.658 | 99 | 1.8 | 1.8 | 96 | 102 |
| 165 | 99.390 | 98.975 | 100.309 | 96.651 | 102.081 | 100.308 | 100 | 1.8 | 1.8 | 97 | 102 |
| 180 | 99.215 | 98.578 | 100.105 | 96.284 | 101.856 | 100.021 | 99 | 1.9 | 1.9 | 96 | 102 |
| 195 | 99.166 | 98.584 | 100.059 | 96.198 | 102.100 | 100.373 | 99 | 2.0 | 2.0 | 96 | 102 |

The dissolution profile for batch LC23120 sub-lot 2 is shown in FIG. 8B.

### Bulk Analysis—Assay and Related Substances

The assay and related substances testing was performed (on n=2 samples) using sub-lot 1 and 2 tablets of bulk batch LC23120 from a representative sample. Results are given in Table 75.

TABLE 75
Assay and related substances results
for batches LC23120 sub-lots 1 and 2.

| | |
|---|---|
| LC23120 Sub-lot 1 | LC23120 Sub-lot 2 |

Assay

| | | |
|---|---|---|
| Sample 1 (% LC) | 99 | 100 |
| Sample 2 (% LC) | 99 | 99 |
| Mean assay (% LC) | 99 | 100 |

Related substances

| | | |
|---|---|---|
| BF4 | <LD | <LD |
| BP2.941 (N-oxide) | <LD | <LD |
| Unknown impurity | <LQ | <LQ |
| Sum of impurities | <LQ | <LQ |

### Release Tests on Film Coated Tablets (HDPE Bottle, 30 Units)

The appearance was performed (on n=10 tablets) from a representative sample of the batch, by visual inspection, on each of the two sub-lots and on packaged bulk. The appearance of the packaged product LC23120A1 complies with specification.

TABLE 76
Appearance results for batch LC23120A1.

| | Specification | LC23120A1 |
|---|---|---|
| Appear- | Purple to light purple round | Purple to light purple round |
| ance | shaped tablet engraved with | shaped tablet engraved with |
| | “C” | “C” |
| | Diameter approx: 7 mm | 7 mm |
| | Thickness: run and record | 4 mm |
| | for information | |

The average mass, water content, disintegration, identification, assay and related substances, content uniformity, and dissolution were tested on the release batch and were substantively the same as bulk testing values provided above and were within the specification, and compliant.

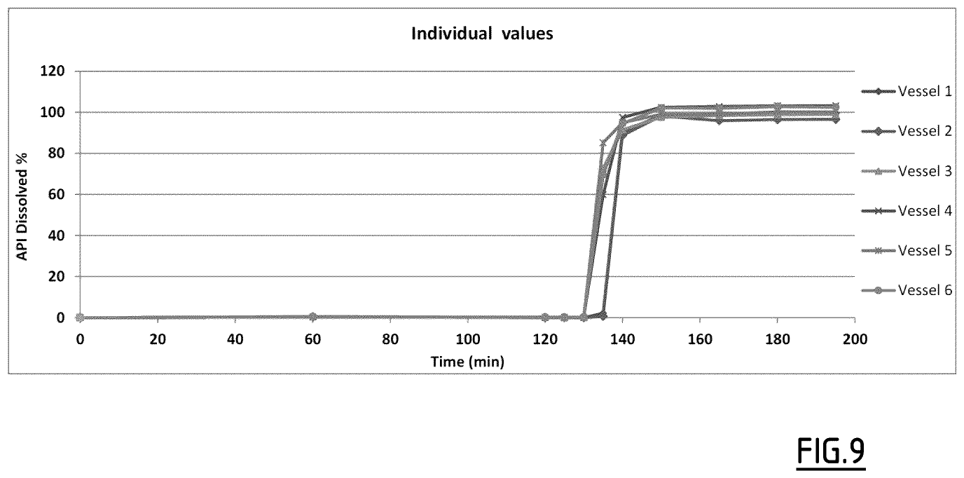

Dissolution profile for batch LC23120A1 is also provided in FIG. 9.

The microbiology testing was performed on tablets from a representative sample, Results comply with specification.

TABLE 77
Microbiology results.

| | Specification | LC23120A1 |
|---|---|---|
| TAMC | ≤10³ CFU/g | <500 CFU/g |
| TYMC | ≤10² CFU/g | <10 CFU/g |
| Specified organism: E. Coli | Absence in 1 g | Absence in 1 g |

TAMC: Total aerobic microbial count
TYMC: Total combined yeasts and moulds count

### CONCLUSION

In conclusion, blending and tableting steps went well. There was a slight difference in color between sub-lots 1 and 2 but no difference according to disintegration and dissolution testing. With regards to analytical IPC, all results were within the specification. No trend was observed on lubricated blend of mix or between start, middle and end of the compression. Results obtained for assay and uniformity of content unit were at the target. In addition, release results all comply with acceptance criteria. Disintegration was recorded only for information.

This manufacturing delivered compliant tablets: pitolisant hydrochloride (20 mg gastro-resistant film-coated tablets corresponding to pitolisant 17.8 mg as free base).

### Example 8. Three-Month Stability Study

The enteric coated dosage forms from Example 7 (batch LC23120A1) were subject to a 3-month stability study. The objectives of the study were to investigate the stability of the product packaged in one packating, and to confirm the shelf-life of the product determined on the supportive batches. Data obtained after 3 months of storage at 5° C., 25° C./60% RH and 40° C./75% RH is summarized below.

TABLE 78
Pitolisant HCl enteric coated dosage forms (made in Example 7).

| | | Unit formula | Centesimal |
|---|---|---|---|
| Raw material | Function | (mg/unit) | formula (%) |
| Pitolisant HCl (BF2.649) | API | 20.00 | 16.00 |
| Cellulose microcrystalline (Vivapur 102) | diluent | 81.00 | 64.80 |
| Crospovidone (Polyplasdone XL) | disintegrant | 10.00 | 8.00 |
| Talc | Glidant | 10.00 | 8.00 |
| Magnesium stearate (Kemilub EM-F-V) | Lubricant | 3.00 | 2.40 |
| Silica colloidal anhydrous (Aerosil 200 pharma) | Glidant | 1.00 | 0.80 |
| TOTAL | / | 125.0 | 100.00 |
| Film-coating | | | |
| Purple Opadry ® AMB II¹ (88A200015) | Coating agent | 3.75 | / |
| ACRYL-EZE ®² clear (93A19346) | Coating agent | 19.3125 | / |
| PEG 8000³ | Plasticizer | 1.545 | / |
| TOTAL | / | 149.61 | / |

¹15% suspension, weight gain = 3%
²20% suspension, weight gain = 15%
³PEG at 8% of acryl eze

TABLE 79
Packaging information.

| | Dosage | | | Number of | Amount of tablets |
|---|---|---|---|---|---|
| Packaged product | strength | Date of | Type of | units per | needed for |
| Batch number | (as salt) | packaging | packaging | bottles | stability |
| LC23120A1 | 20 mg | Jul. 5^{th}, 2023 | HDPE bottle | 30 | 8400 |
| | | | and PP cap | | |
| | | | fitted with | | |
| | | | 2.4 g desiccant | | |

TABLE 80
Packaging articles characteristics.

| Packaging type | Characteristics |
|---|---|
| HDPE bottle and | 20 mL White HDPE bottle and white tamper evident |
| PP caps | childproof PP caps with desiccant (2.4 g) |

Stability Program. Storage Conditions and Stability Testing

The storage of samples is performed according to ICH conditions: Long term conditions 25° C./60% RH; Intermediate conditions 30° C./65% RH; Accelerated conditions 40° C./75% RH; 5° C. for information.

TABLE 81
Storage conditions and stability testing

| Storage | Test times (months) | | | | | | | | | | |
|---|---|---|---|---|---|---|---|---|---|---|---|
| conditions | 0.5 | 1 | 2 | 3 | 6 | 9 | 12 | 18 | 24 | 36 | Back-up |
| 5° C. | 1 | 1 | [1] | 1 | 1 | 1 | 1 + 2 | 1 | 1 + 2 | 1 + 2 | 1 + 2 |
| 25° C./60% RH | 1 | 1 | [1] | 1 | 1 | 1 | 1 + 2 | 1 | 1 + 2 | 1 + 2 | 1 + 2 |
| 30° C./65% RH | [1]¹ | [1]¹ | [1]¹ | [1]¹ | [1]¹ | [1]¹ | [1 + 2]¹ | — | — | — | [1 + 2]¹ |
| 40° C./75% RH | 1 | 1 | [1] | 1 | 1 + 2 | — | — | — | — | — | 1 + 2 |

—: not tested;
[ ]: optional testing;
¹only if 40° C./75% RH condition fails

After removal from climatic chambers, all samples were stored at 15-25° C. before and after analysis.

### Pitolisant Hydrochloride 20 mg, Gastro Resistant Film-Coated Tablets, Batch LC23120A1 After 3 Months Of Storage at 5° C., 25° C./60% RH and 40° C./75% RH

Appearance: No change in appearance was observed after 3 months at 5° C., 25° C./60% RH and 40° C./75% RH. The appearance complies with the specification.

Mean mass: A slight decrease in mean mass was observed after 3 months at 5° C. and 25° C./60% RH (respectively from 148.0 mg at initial to 146.9 mg, and 147.0 mg). No significant change in mean mass was observed after 3 months at 40° C./75% RH. The mean mass results comply with the specification.

Water content: A slight increase was observed in water content results after 3 months for all conditions, whereas a slight decrease was observed after 2 months at 25° C./60% RH and 40° C./75% RH.

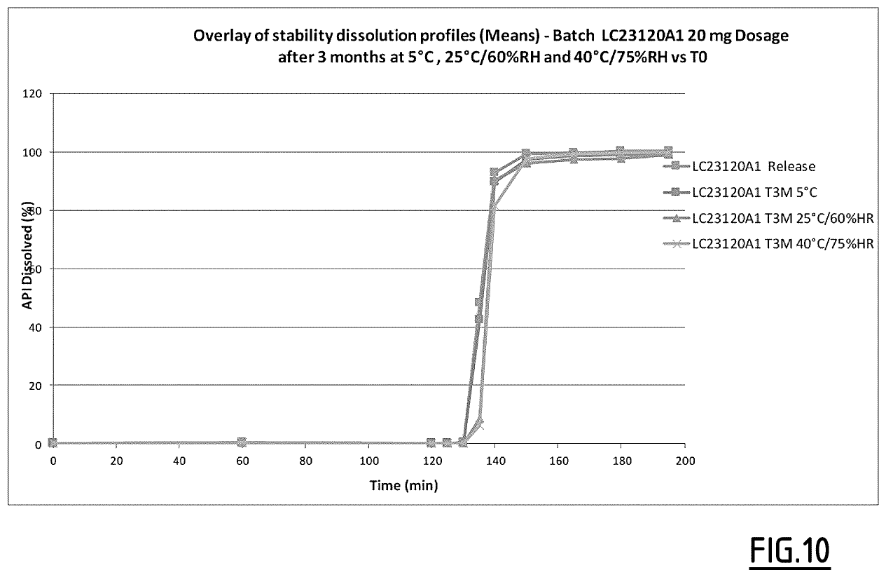

Dissolution: After 3 months at 25° C./60% RH and 40° C./75% RH, a slightly slower dissolution profile was observed at the start of dissolution in buffer stage. At 5° C., no significant change was observed. The dissolution results comply with the specification. An overlay of stability dissolution profiles is provide in FIG. 10.

Disintegration time: After 3 months of storage at 5° C., no significant change was observed for the disintegration time. An increase was observed after 1 month of storage at 25° C./60% RH and 40° C./75% RH. At the 3 month time point, the disintegration was about 3 to 4 minutes longer than the initial timepoint and shows variation tablet to tablet.

Assay/Purity: Regarding the assay, no significant change was observed after 3 months at 5° C., 25° C./60% RH and 40° C./75% RH. The assay results comply with the specification.

Regarding the purity, all the individual impurities as well as the total of impurities remained<0.05%. The purity results comply with the specification.

Microbiology testing: At initial testing, the microbial results were within the specification.

Conclusion: After 3 months storage at 5° C., 25° C./60% RH and 40° C./75% RH in HDPE bottle, the 20 mg formulation of pitolisant hydrochloride gastro-resistant film-coated tablets, corresponding to pitolisant 17.8 mg free base, packaged in 20 mL HDPE bottles (30 tablets per bottle) closed with PP cap fitted with desiccant 2.4 g is stable. Based on the stability data obtained, the current 12 months shelf-life applied to the pitolisant hydrochloride 20 mg gastro-resistant film-coated tablets is compliant and stable across all storage conditions.

### EQUIVALENTS

Those skilled in the art will recognize or be able to ascertain using no more than routine experimentation many equivalents to the specific embodiments disclosed herein. Those of ordinary skill in the art will appreciate that various changes or modifications to this description may be made without departing from the spirit or scope of the present disclosure, as defined in the following claims.

1. An oral dosage form comprising:
a core that comprises pitolisant monohydrochloride and one or more pharmaceutically acceptable excipients;
an anti-moisture barrier that surrounds the core; and
an enteric coating that surrounds the core and the anti-moisture barrier.

2-4. (canceled)

5. The oral dosage form of claim 1, wherein the enteric coating comprises ACRYL-EZE®.

6. The oral dosage form of claim 5, wherein the enteric coating further comprises a plasticizer.

7. The oral dosage form of claim 6, wherein the plasticizer comprises polyethylene glycol (PEG).

8-9. (canceled)

10. The oral dosage form of claim 1, wherein the anti-moisture barrier comprises a polyvinyl alcohol (PVA)-based polymer.

11. The oral dosage form of claim 1, wherein the anti-moisture barrier comprises OPADRY® amb II.

12. The oral dosage form of claim 1, comprising about 20 mg of pitolisant monohydrochloride.

13. The oral dosage form of claim 1, comprising about 5 mg of pitolisant monohydrochloride.

14-15. (canceled)

16. The oral dosage form of claim 1, wherein the one or more pharmaceutically acceptable excipients are selected from the group consisting of microcrystalline cellulose, crospovidone, talc, magnesium stearate, and colloidal silica.

17-22. (canceled)

23. The oral dosage form of claim 1, wherein the pitolisant monohydrochloride is crystalline and has an X-ray diffractogram that comprises characteristic peaks (2θ) at 11.2°, 19.9°, 20.7°, and 34.1° (±0.2°).

24-26. (canceled)

27. The oral dosage form of claim 1, wherein the oral dosage form is a tablet.

28. The oral dosage form of claim 1, wherein the oral dosage form is bioequivalent to another oral dosage form comprising pitolisant monohydrochloride in the same amount, and wherein the another oral dosage form does not comprise an anti-moisture barrier and an enteric coating.

29. The oral dosage form of claim 1, wherein orally administering the oral dosage form to a subject provides a Cₘₐₓ of pitolisant in the subject that is substantially the same as the Cₘₐₓ of pitolisant provided by orally administering another oral dosage form to a subject comprising pitolisant monohydrochloride in the same amount, wherein the another oral dosage form does not comprise an anti-moisture barrier and an enteric coating.

30. The oral dosage form of claim 1, wherein orally administering the oral dosage form once daily to a subject for a period of about 7 days provides a steady state Cₘₐₓ of pitolisant that is substantially the same as the steady state Cₘₐₓ of pitolisant following orally administering once daily to a subject for a period of about 7 days another oral dosage form comprising pitolisant monohydrochloride in the same amount, wherein the another oral dosage form does not comprise an anti-moisture barrier and an enteric coating.

31. The oral dosage form of claim 1, wherein orally administering the oral dosage form to a subject provides an AUC of pitolisant that is substantially the same as the AUC of pitolisant obtained following orally administering another oral dosage form to a subject comprising pitolisant monohydrochloride in the same amount, wherein the another oral dosage form does not comprise an anti-moisture barrier and an enteric coating.

32. The oral dosage form of claim 1, wherein orally administering the oral dosage form once daily to a subject for a period of about 7 days provides a steady state AUC of pitolisant that is substantially the same as the steady state AUC of pitolisant following orally administering once daily to a subject for a period of about 7 days another oral dosage form comprising pitolisant monohydrochloride in the same amount, wherein the another oral dosage form does not comprise an anti-moisture barrier and an enteric coating.

33. The oral dosage form of claim 1, wherein orally administering the oral dosage form to a subject provides a Tₘₐₓ of pitolisant that is substantially the same as the Tₘₐₓ of pitolisant obtained following orally administering to a subject another oral dosage form comprising pitolisant monohydrochloride in the same amount, wherein the another oral dosage form does not comprise an anti-moisture barrier and an enteric coating.

34. A method of treating excessive daytime sleepiness (EDS) or cataplexy, comprising orally administering to a subject in need thereof an oral dosage form of claim 1.

35-38. (canceled)

39. The method of claim 34, wherein the subject has narcolepsy.

40-43. (canceled)

44. An oral dosage form comprising a tablet, wherein the tablet comprises:
a core that comprises crystalline pitolisant monohydrochloride and one or more pharmaceutically acceptable excipients;
an anti-moisture barrier that surrounds the core, wherein the anti-moisture barrier comprises an OPADRY® polymer; and
an enteric coating that surrounds the core and the anti-moisture barrier, wherein the enteric coating comprises ACRYL-EZE® and a plasticizer.

45. The oral dosage form of claim 44, wherein the anti-moisture barrier comprises OPADRY® amb II.

46-57. (canceled)

58. The oral dosage form of claim 44, wherein the plasticizer comprises polyethylene glycol (PEG).

59. The oral dosage form of claim 44, comprising about 20 mg of pitolisant monohydrochloride.

60. The oral dosage form of claim 44, comprising about 5 mg of pitolisant monohydrochloride.

61. The oral dosage form of claim 44, wherein the pitolisant monohydrochloride is crystalline and has an X-ray diffractogram that comprises characteristic peaks (2θ) at 11.2°, 19.9°, 20.7°, and 34.1° (±0.2°).

62. The oral dosage form of claim 1, wherein the oral dosage form is bioequivalent to a WAKIX″ tablet comprising the same amount of pitolisant monohydrochloride.

63. The oral dosage form of claim 44, wherein the oral dosage form is bioequivalent to a WAKIX® tablet comprising the same amount of pitolisant monohydrochloride.

64. The oral dosage form of claim 44, wherein the one or more pharmaceutically acceptable excipients are selected from the group consisting of microcrystalline cellulose, crospovidone, talc, magnesium stearate, and colloidal silica.

65. A method of treating excessive daytime sleepiness (EDS) or cataplexy, comprising orally administering to a subject in need thereof an oral dosage form of claim 44.

66. The method of claim 65, wherein the subject has narcolepsy.
